SEQUENCE LISTING (8 sequences)

SEQ ID NO: 1 - DNA, 6774 bp, Homo sapiens
cggccccagaaaacccgagcgagtagggggcggcgcgcaggagggaggagaactgggggcgcgggaggctggtgggtgtggggggtggagatgtagaagatgtgacgccgcggcccggcgggtgccagattagcggacgcggtgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctgattttaatggccacatctaatctcatttcacatgaaagaagaagtatattttagaaatttgttaatgagagtaaaagaaaataaatgtgtatagctcagtttggataattggtcaaacaattttttatccagtagtaaaatatgtaaccattgtcccagtaaagaaaaataacaaaagttgtaaaatgtatattctcccttttatattgcatctgctgttacccagtgaagcttacctagagcaatgatctttttcacgcatttgctttattcgaaaagaggcttttaaaatgtgcatgtttagaaacaaaatttcttcatggaaatcatatacattagaaaatcacagtcagatgtttaatcaatccaaaatgtccactatttcttatgtcattcgttagtctacatgtttctaaacatataaatgtgaatttaatcaattcctttcatagttttataattctctggcagttccttatgatagagtttataaaacagtcctgtgtaaactgctggaagttcttccacagtcaggtcaattttgtcaaacccttctctgtacccatacagcagcagcctagcaactctgctggtgatgggagttgtattttcagtcttcgccaggtcattgagatccatccactcacatcttaagcattcttcctggcaaaaatttatggtgaatgaatatggctttaggcggcagatgatatacatatctgacttcccaaaagctccaggatttgtgtgctgttgccgaatactcaggacggacctgaattctgattttataccagtctcttcaaaaacttctcgaaccgctgtgtctcctacgtaaaaaaagagatgtacaaatcaataataattacacttttagaaactgtatcatcaaagattttcagttaaagtagcattatgtaaaggctcaaaacattaccctaacaaagtaaagttttcaatacaaattctttgccttgtggatatcaagaaatcccaaaatattttcttaccactgtaaattcaagaagcttttgaaatgctgaatatttctttggctgctacttggaggcttatctacctgtacatttttggggtcagctctttttaacttcttgctgctctttttcccaaaaggtaaaaatatagattgaaaagttaaaacattttgcatggctgcagttcctttgtttcttgagataagattccaaagaacttagattcatttcttcaacaccgaaatgctggaggtgtttgatcagttttcaagaaacttggaatataaataattttataattcaacaaaggttttcacattttataaggttgatttttcaattaaatgcaaatttgtgtggcaggatttttattgccattaacatatttttgtggctgctttttctacacatccagatggtccctctaactgggctttctctaattttgtgatgttctgtcattgtctcccaaagtatttaggagaagccctttaaaaagctgccttcctctaccactttgctggaaagcttcacaattgtcacagacaaagatttttgttccaatactcgttttgcctctatttttcttgtttgtcaaatagtaaatgatatttgcccttgcagtaattctactggtgaaaaacatgcaaagaagaggaagtcacagaaacatgtctcaattcccatgtgctgtgactgtagactgtcttaccatagactgtcttacccatcccctggatatgctcttgttttttccctctaatagctatggaaagatgcatagaaagagtataatgttttaaaacataaggcattcgtctgccatttttcaattacatgctgacttcccttacaattgagatttgcccataggttaaacatggttagaaacaactgaaagcataaaagaaaaatctaggccgggtgcagtggctcatgcctatattccctgcactttgggaggccaaagcaggaggatcgcttgagcccaggagttcaagaccaacctggtgaaaccccgtctctacaaaaaaacacaaaaaatagccaggcatggtggcgtgtacatgtggtctcagatacttgggaggctgaggtgggagggttgatcacttgaggctgagaggtcaaggttgcagtgagccataatcgtgccactgcagtccagcctaggcaacagagtgagactttgtctcaaaaaaagagaaattttccttaataagaaaagtaatttttactctgatgtgcaatacatttgttattaaatttattatttaagatggtagcactagtcttaaattgtataaaatatcccctaacatgtttaaatgtccatttttattcattatgctttgaaaaataattatggggaaatacatgtttgttattaaatttattattaaagatagtagcactagtcttaaatttgatataacatctcctaacttgtttaaatgtccatttttattctttatgtttgaaaataaattatggggatcctatttagctcttagtaccactaatcaaaagttcggcatgtagctcatgatctatgctgtttctatgtcgtggaagcaccggatgggggtagtgagcaaatctgccctgctcagcagtcaccatagcagctgactgaaaatcagcactgcctgagtagttttgatcagtttaacttgaatcactaactgactgaaaattgaatgggcaaataagtgcttttgtctccagagtatgcgggagacccttccacctcaagatggatatttcttccccaaggatttcaagatgaattgaaatttttaatcaagatagtgtgctttattctgttgtattttttattattttaatatactgtaagccaaactgaaataacatttgctgttttataggtttgaagaacataggaaaaactaagaggttttgtttttatttttgctgatgaagagatatgtttaaatatgttgtattgttttgtttagttacaggacaataatgaaatggagtttatatttgttatttctattttgttatatttaataatagaattagattgaaataaaatataatgggaaataatctgcagaatgtgggttttcctggtgtttccctctgactctagtgcactgatgatctctgataaggctcagctgctttatagttctctggctaatgcagcagatactcttcctgccagtggtaatacgattttttaagaaggcagtttgtcaattttaatcttgtggatacctttatactcttagggtattattttatacaaaagccttgaggattgcattctattttctatatgaccctcttgatatttaaaaaacactatggataacaattcttcatttacctagtattatgaaagaatgaaggagttcaaacaaatgtgtttcccagttaactagggtttactgtttgagccaatataaatgtttaactgtttgtgatggcagtattcctaaagtacattgcatgttttcctaaatacagagtttaaataatttcagtaattcttagatgattcagcttcatcattaagaatatcttttgttttatgttgagttagaaatgccttcatatagacatagtctttcagacctctactgtcagttttcatttctagctgctttcagggttttatgaattttcaggcaaagctttaatttatactaagcttaggaagtatggctaatgccaacggcagtttttttcttcttaattccacatgactgaggcatatatgatctctgggtaggtgagttgttgtgacaaccacaagcactttttttttttttaaagaaaaaaaggtagtgaatttttaatcatctggactttaagaaggattctggagtatacttaggcctgaaattatatatatttggcttggaaatgtgtttttcttcaattacatctacaagtaagtacagctgaaattcagaggacccataagagttcacatgaaaaaaatcaatttatttgaaaaggcaagatgcaggagagaggaagccttgcaaacctgcagactgctttttgcccaatatagattgggtaaggctgcaaaacataagcttaattagctcacatgctctgctctcacgtggcaccagtggatagtgtgagagaattaggctgtagaacaaatggccttctctttcagcattcacaccactacaaaatcatcttttatatcaacagaagaataagcataaactaagcaaaaggtcaataagtacctgaaaccaagattggctagagatatatcttaatgcaatccattttctgatggattgttacgagttggctatataatgtatgtatggtattttgatttgtgtaaaagttttaaaaatcaagctttaagtacatggacatttttaaataaaatatttaaagacaatttagaaaattgccttaatatcattgttggctaaatagaataggggacatgcatattaaggaaaaggtcatggagaaataatattggtatcaaacaaatacattgatttgtcatgatacacattgaatttgatccaatagtttaaggaataggtaggaaaatttggtttctatttttcgatttcctgtaaatcagtgacataaataattcttagcttattttatatttccttgtcttaaatactgagctcagtaagttgtgttaggggattatttctcagttgagactttcttatatgacattttactatgttttgacttcctgactattaaaaataaatagtagatacaattttcataaagtgaagaattatataatcactgctttataactgactttattatatttatttcaaagttcatttaaaggctactattcatcctctgtgatggaatggtcaggaatttgttttctcatagtttaattccaacaacaatattagtcgtatccaaaataacctttaatgctaaactttactgatgtatatccaaagcttctcattttcagacagattaatccagaagcagtcataaacagaagaataggtggtatgttcctaatgatattatttctactaatggaataaactgtaatattagaaattatgctgctaattatatcagctctgaggtaatttctgaaatgttcagactcagtcggaacaaattggaaaatttaaatttttattcttagctataaagcaagaaagtaaacacattaatttcctcaacatttttaagccaattaaaaatataaaagatacacaccaatatcttcttcaggctctgacaggcctcctggaaacttccacatatttttcaactgcagtataaagtcagaaaataaagttaacataactttcactaacacacacatatgtagatttcacaaaatccacctataattggtcaaagtggttgagaatatattttttagtaattgcatgcaaaatttttctagcttccatcctttctccctcgtttcttctttttttgggggagctggtaactgatgaaatcttttcccaccttttctcttcaggaaatataagtggttttgtttggttaacgtgatacattctgtatgaatgaaacattggagggaaacatctactgaatttctgtaatttaaaatattttgctgctagttaactatgaacagatagaagaatcttacagatgctgctataaataagtagaaaatataaatttcatcactaaaatatgctattttaaaatctatttcctatattgtatttctaatcagatgtattactcttattatttctattgtatgtgttaatgattttatgtaaaaatgtaattgcttttcatgagtagtatgaataaaattgattagtttgtgttttcttgtctccc

SEQ ID NO: 2 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 3 - DNA, 4704 bp, Homo sapiens
ggagagggagaaggctctcgggcggagagaggtcctgcccagctgttggcgaggagtttcctgtttcccccgcagcgctgagttgaagttgagtgagtcactcgcgcgcacggagcgacgacacccccgcgcgtgcacccgctcgggacaggagccggactcctgtgcagcttccctcggccgccgggggcctccccgcgcctcgccggcctccaggccccctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatagctcttctggggcaggctgggccatgtccaaagaggctgcccctctcaccaaagaacagaggcagcaggaagctgcccctgaactgatgcttcctggaaaaccaagggggtcactcccctccctgtaagctgtggggataagcagaaacaacagcagcagggagtgggtgacatagagcattctatgcctttgacattgtcataggataagctgtgttagcacttcctcaggaaatgagattgatttttacaatagccaataacatttgcactttattaatgcctgtatataaatatgaatagctatgttttatatatatatatatatatctatatatgtctatagctctatatatatagccataccttgaaaagagacaaggaaaaacatcaaatattcccaggaaattggttttattggagaactccagaaccaagcagagaaggaagggacccatgacagcattagcatttgacaatcacacatgcagtggttctctgactgtaaaacagtgaactttgcatgaggaaagaggctccatgtctcacagccagctatgaccacattgcacttgcttttgcaaaataatcattccctgcctagcacttctcttctggccatggaactaagtacagtggcactgtttgaggaccagtgttcccggggttcctgtgtgcccttatttctcctggacttttcatttaagctccaagccccaaatctggggggctagtttagaaactctccctcaacctagtttagaaactctaccccatctttaataccttgaatgttttgaaccccactttttaccttcatgggttgcagaaaaatcagaacagatgtccccatccatgcgattgccccaccatctactaatgaaaaattgttctttttttcatctttcccctgcacttatgttactattctctgctcccagccttcatccttttctaaaaaggagcaaattctcactctaggctttatcgtgtttactttttcattacacttgacttgattttctagttttctatacaaacaccaatgggttccatctttctgggctcctgattgctcaagcacagtttggcctgatgaagaggatttcaactacacaatactatcattgtcaggactatgacctcaggcactctaaacatatgttttgtttggtcagcacagcgtttcaaaaagtgaagccactttataaatatttggagattttgcaggaaaatctggatccccaggtaaggatagcagatggttttcagttatctccagtccacgttcacaaaatgtgaaggtgtggagacacttacaaagctgcctcacttctcactgtaaacattagctctttccactgcctacctggaccccagtctaggaattaaatctgcacctaaccaaggtcccttgtaagaaatgtccattcaagcagtcattctctgggtatataatatgattttgactaccttatctggtgttaagatttgaagttggccttttattggactaaaggggaactcctttaagggtctcagttagcccaagtttcttttgcttatatgttaatagttttaccctctgcattggagagaggagtgctttactccaagaagctttcctcatggttaccgttctctccatcatgccagccttctcaacctttgcagaaattactagagaggatttgaatgtgggacacaaaggtcccatttgcagttagaaaatttgtgtccacaaggacaagaacaaagtatgagctttaaaactccataggaaacttgttaatcaacaaagaagtgttaatgctgcaagtaatctcttttttaaaactttttgaagctacttattttcagccaaataggaatattagagagggactggtagtgagaatatcagctctgtttggatggtggaaggtctcattttattgagatttttaagatacatgcaaaggtttggaaatagaacctctaggcaccctcctcagtgtgggtgggctgagagttaaagacagtgtggctgcagtagcatagaggcgcctagaaattccacttgcaccgtagggcatgctgataccatcccaatagctgttgcccattgacctctagtggtgagtttctagaatactggtccattcatgagatattcaagattcaagagtattctcacttctgggttatcagcataaactggaatgtagtgtcagaggatactgtggcttgttttgtttatgtttttttttcttattcaagaaaaaagaccaaggaataacattctgtagttcctaaaaatactgacttttttcactactatacataaagggaaagttttattcttttatggaacacttcagctgtactcatgtattaaaataggaatgtgaatgctatatactctttttatatcaaaagtctcaagcacttatttttattctatgcattgtttgtcttttacataaataaaatgtttattagattgaataaagcaaaatactcaggtgagcatcctgcctcctgttcccattcctagtagctaaa

SEQ ID NO: 4 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 5 - DNA, 6105 bp, Homo sapiens
agtacagtataaaacttcacagtgccaataccatgaagaggagctcagacagctcttaccacatgatacaagagccggctggtggaagagtggggaccagaaagagaatttgctgaagaggagaaggaaaaaaaaaacaccaaaaaaaaaaataaaaaaatccacacacacaaaaaaacctgcgcgtgaggggggaggaaaagcagggccttttaaaaaggcaatcacaacaacttttgctgccaggatgcccttgctttggctgagaggatttctgttggcaagttgctggattatagtgaggagttcccccaccccaggatccgaggggcacagcgcggcccccgactgtccgtcctgtgcgctggccgccctcccaaaggatgtacccaactctcagccagagatggtggaggccgtcaagaagcacattttaaacatgctgcacttgaagaagagacccgatgtcacccagccggtacccaaggcggcgcttctgaacgcgatcagaaagcttcatgtgggcaaagtcggggagaacgggtatgtggagatagaggatgacattggaaggagggcagaaatgaatgaacttatggagcagacctcggagatcatcacgtttgccgagtcaggaacagccaggaagacgctgcacttcgagatttccaaggaaggcagtgacctgtcagtggtggagcgtgcagaagtctggctcttcctaaaagtccccaaggccaacaggaccaggaccaaagtcaccatccgcctcttccagcagcagaagcacccgcagggcagcttggacacaggggaagaggccgaggaagtgggcttaaagggggagaggagtgaactgttgctctctgaaaaagtagtagacgctcggaagagcacctggcatgtcttccctgtctccagcagcatccagcggttgctggaccagggcaagagctccctggacgttcggattgcctgtgagcagtgccaggagagtggcgccagcttggttctcctgggcaagaagaagaagaaagaagaggagggggaagggaaaaagaagggcggaggtgaaggtggggcaggagcagatgaggaaaaggagcagtcgcacagacctttcctcatgctgcaggcccggcagtctgaagaccaccctcatcgccggcgtcggcggggcttggagtgtgatggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctcatagagttgcccagcccagggggaaagggagcaagagttgtccagagaagacagtggcaaaatgaagaaatttttaaggtttctgagttaaccagaaaaatagaaattaaaaacaaaacaaaaaaaaaaacaaaaaaaaacaaaagtaaattaaaaacaaaacctgatgaaacagatgaaggaagatgtggaaaaaatccttagccagggctcagagatgaagcagtgaaagagacaggaattgggagggaaagggagaatggtgtaccctttatttcttctgaaatcacactgatgacatcagttgtttaaacggggtattgtcctttccccccttgaggttcccttgtgagccttgaatcaaccaatctagtctgcagtagtgtggactagaacaacccaaatagcatctagaaagccatgagtttgaaagggcccatcacaggcactttcctacccaattacccaggtcataaggtatgtctgtgtgacacttatctctgtgtatatcagcatacacacacacacacacacacacacacacacacacaggcatttccacacattacatatatacacatactggtaaaagaacaatcgtgtgcaggtggtcacacttcctttttctgtaccacttttgcaacaaaacaaaacaaacaacattaaaaaattgagaacaagtatggaaagaatgaaagatcaaggaaaaaagaataccaagttacatttcgttaaggtgcttatgatcttagaactatgcaacctaataggtttgaaactgtttacctgagagagaacaaaaagagagacttttttgtattggaagtaatctgattaatttttattttcttcaaggagagatacttgaaaggaatatgtttgtccatctgttggatccaaacatttctatattttgtaaatgttgttgttgttttttttttaatcgtttactatttgcactacaatggtgtttgacctgtctaatccttatttaacaagtattttctttggttgggggtgggggtggggtttaagagctgcacttaatgtgagctataaaagaactgctacagcacacaaaatagctatttttattattataattataattattattattattttgtaccttaaaaaatagacacatacaccaaagacatttgtgtgagcctttaaacagtctgtctgtggttggtatcattcaccatcaatgagtcaggggttgggattcaaggttgagtagtgtggattgtgttcaggcttaaaagacctgagaagtttggtttttgactccttttacatccatgaaacaggacatttcatactggatgtacagtagttgtacactgttggatatcaagttcaatcaaattcatggaactacatgcttgtatgtgtatatatacattgcttgtgcatatgcatatctgtatgtatatatacatgtattgtaccatgtccatacacattttaagcacttcaggctgtcattttttaatgttcttaaagcaatgaatgtttgtgtgcaaaacacagtatttttaagaaggataggctatagtttttgcttttactctgaactaggtgggcgcatttcaaaaattcggatgggaaaaagcctggaaattccagtgaatattcagcaaggccctctttcattgtacagggatcaaatttcctcctcttttttgtgccccctcccacttctacaagttatcccctgtggggaaaacaggatgataatcaaaactctgggctgatgtttttccaacttagtgtctattggaatcaatcttaaatcagaagctttttcagaaaaataatatttaggccagaattagagttgagtgtattttttaaaaatgattaaggcttggttgtgagaaatattacctgtaccagctgggaaaaataatgtcatcactaactaaaagataattaatttgagagaaagtgttaagagagggagagtaaggaagagaacagttaagaggaggcagaggtgagggcagtagtaaaaatctctaaaattttaatttacagccaaaattcttcatgtgtaaatttgtattgattcagatgcagaaatgaaaaaaaaacacctttgttttataaatatcaaagtacatgcttaaagccaagtttttatctagtttattctagtacttagcttgcctggaatagctaataaattattcatgtatgtgcttttgaaaatccagagccctatttttacacacttgtgtgaagttggcaaacattttgaaaaatggaaaaaagtttctaataattgggaacaattacattaattaatattttgtaaaatattgaagcttttagccctatgtcaatttgtagattaaaataaattaattataggaaaggaagataacagtgagaaaccaaacattacaaaaggtggtttagctctccttgaaaaatatactaagttggtatactataacacttggctatatgtaggcaatgtcactactgggcaaatacacttactgtgttctagaggcagccctttcttatgcagaaaatacaatacgcactgcatgagaagcttgagagtggattctaatccaggtctgtcgaccttggatatcatgcatgtgggaaggtgggtgtggtgagaaaagttttaaggcaagagtagatggccatgttcaactttacaaaatttcttggaaaactggcagtattttgaactgcatcttctttggtaccggaacctgcagaaacagtgtgagaaattaagtcctggttcactgcgcagtagcaaagatggtcaaggccatggaaaaagcagaaatttaccaagaaagctgatacccatgtatagttcccactcatctcaaatacatctgctatctttttaagctaagtcctagacatatcggggataacatgggggttgattagtgaccacagttatcagaagcagagaaatgtaattccatattttatttgaaacttattccatattttaattggatattgagtgattgggttatcaaacacccacaaactttaattttgttaaatttatatggctttgaaatagaagtataagttgctaccattttttgataacattgaaagatagtattttaccatctttaatcatcttggaaaatacaagtcctgtgaacaaccactctttcacctagcagcatgaggccaaaagtaaaggctttaaattataacatatgggattcttagtagtatgtttttttcttgaaactcagtggctctatctaaccttactatctcctcactctttctctaagactaaactctaggctcttaaaaatctgcccacaccaatcttagaagctctgaaaagaatttgtctttaaatatcttttaatagtaacatgtattttatggaccaaattgacattttcgactattttttccaaaaaagtcaggtgaatttcagcacactgagttgggaatttcttatcccagaagaccaaccaatttcatatttatttaagattgattccatactccgttttcaaggagaatccctgcagtctccttaaaggtagaacaaatactttctatttttttttcaccattgtgggattggactttaagaggtgactctaaaaaaacagagaacaaatatgtctcagttgtattaagcacggacccatattatcatattcacttaaaaaaatgatttcctgtgcaccttttggcaacttctcttttcaatgtagggaaaaacttagtcaccctgaaaacccacaaaataaataaaacttgtagatgtgggcagaaggtttgggggtggacattgtatgtgtttaaattaaaccctgtatcactgagaagctgttgtatgggtcagagaaaatgaatgcttagaagctgttcacatcttcaagagcagaagcaaaccacatgtctcagctatattattatttattttttatgcataaagtgaatcatttcttctgtattaatttccaaagggttttaccctctatttaaatgctttgaaaaacagtgcattgacaatgggttgatatttttctttaaaagaaaaatataattatgaaagccaagataatctgaagcctgttttattttaaaactttttatgttctgtggttgatgttgtttgtttgtttgtttctattttgttggttttttactttgttttttgttttgttttgttttgttttgcatactacatgcagttctttaaccaatgtctgtttggctaatgtaattaaagttgttaatttatatgagtgcatttcaactatgtcaatggtttcttaatatttattgtgtagaagtactggtaatttttttatttacaatatgtttaaagagataacagtttgatatgttttcatgtgtttatagcagaagttatttatttctatggcattccagcggatattttggtgtttgcgaggcatgcagtcaatattttgtacagttagtggacagtattcagcaacgcctgatagcttctttggccttatgttaaataaaaagacctgtttgggatgtaaaaaaaaaaaaaaaaaaaaaaaaaaaaaaaaaaaaaaaaaa

SEQ ID NO: 6 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 7 - DNA, 2988 bp, Homo sapiens
gcaggagggcccagcgacgccgccgcgccagctcccagggcccggccccccccggcgctcacgctctcggggcggactcccggccctccgcgccctctcgcgcggcgatggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagtaggcaccggccgcggctccccctggacggggcgggccctgcctgagggtgggcttttccctgggtggagcaggactcccacctaaacggggcagtactcctccctgggggcgggactcctccctgggggtggggctcctacctgggggcagaactcctacctgaaggcagggctcctccctggagctagtgtctcctctctggtggctgggctgctcctgaatgaggcggagctccaggatggggaggggctctgcgttggcttctccctggggacggggctcccctggacagaggcggggctacagattgggcggggcttctcttgggtgggacagggcttctcctgcgggggcgaggcccctcccagtaagggcgtggctctgggtgggcggggcactaggtaggcttctacctgcaggcggggctcctcctgaaggaggcggggctctaggatggggcacggctctggggtaggctgctccctgagggcggagcgcctccttaggagtggggttttatggtggatgaggcttcttcctggatggggcagagcttctcctgaccagggcaaggccccttccacgggggctgtggctctgggtgggcgtggcctgcataggctccttcctgtgggtggggcttctctgggaccaggctccaatggggcggggcttctctccgcgggtgggactcttccctgggaaccgccctcctgattaaggcgtggcttctgcaggaatcccggctccagagcaggaaattcagcccaccagccacctcatccccaaccccctgtaaggttccatccacccctgcgtcgagctgggaaggttccatgaagcgagtcgggtccccaacccgtgcccctgggatccgagggcccctctccaagcgcctggctttggaatgctccaggcgcgccgacgcctgtgccaccccttcctcagcctggggtttgaccacccacctgaccaggggccctacctggggaaagcctgaagggcctcccagcccccaaccccaagaccaagcttagtcctgggagaggacagggacttcgcagaggcaagcgaccgaggccctcccaaagaggcccgccctgcccgggctcccacaccgtcaggtactcctgccagggaactggcctgctgcgccccaggccccgcccgtctctgctctgctcagctgcgcccccttctttgcagctgcccagcccctcctccctgccctcgggtctccccacctgcactccatccagctacaggagagatagaagcctctcgtcccgtccctccctttcctccgcctgtccacagccccttaagggaaaggtaggaagagaggtccagccccccaggctgcccagagctgctggtctcatttgggggcgttcgggaggtttggggggcatcaaccccccgactgtgctgctcgcgaaggtcccacagccctgagatgggccggcccccttcctggcccctcatggcgggactggagaaatggtccgctttcctggagccaatggcccggcccctcctgactcatccgcctggcccgggaatgaatggggaggccgctgaacccacccggcccatatccctggttgcctcatggccagcgcccctcagcctctgccactgtgaaccggctcccaccctcaaggtgcggggagaagaagcggccaggcggggcgccccaagagcccaaaagagggcacaccgccatcctctgcctcaaattctgcgtttttggttttaatgttatatctgatgctgctatatccactgtccaacggagttagacgaaaaaaaaaaaaaaaaaa

SEQ ID NO: 8 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

(19) United States
(12) Patent Application Publication
(10) Pub. No.: US 2023/0392125 A1
(43) Pub. Date: Dec. 7, 2023

(54) METHODS FOR GENERATING FORMATIVE PLURIPOTENT STEM CELLS COMPETENT FOR DIRECT PRIMORDIAL GERM CELL INDUCTION

(71) Applicant: The Board of Regents of The University of Texas System, Austin, TX (US)

(72) Inventors: Jun WU, Dallas, TX (US); Leqian YU, Dallas, TX (US)

(73) Assignee: The Board of Regents of The University of Texas System, Austin, TX (US)

(21) Appl. No.: 18/249,661

(22) PCT Filed: Oct. 19, 2021
(86) PCT No.: PCT/US2021/055604

§ 371 Date: Apr. 19, 2023

Related U.S. Application Data
(60) Provisional application No. 63/093,680, filed on Oct. 19, 2020.

(51) Int. Cl. C12N 5/074; C12N 5/0735; C12N 15/85

(52) U.S. Cl. CPC C12N 5/0696; C12N 5/0606; C12N 15/85; C12N 2501/115; C12N 2501/15; C12N 2501/415; C12N 2501/16; C12N 2501/603; C12N 2501/602; C12N 2501/235; C12N 2800/107

(57) ABSTRACT

Aspects of the present disclosure relate to methods for producing formative pluripotent stem cells (PSCs) by culturing cells in a medium that comprises a fibroblast growth factor (FGF) activator, a transforming growth factor beta (TGF-β) activator, and a WNT activator. Also provided are in vitro culture systems for producing the formative pluripotent stem cells.

### CROSS-REFERENCE TO RELATED APPLICATIONS

This application claims priority from Provisional Application No. 63/093,680, filed Oct. 19, 2020, the entire contents of which are hereby incorporated by reference.

### SEQUENCE LISTING

This application contains a Sequence Listing that has been submitted in ASCII format via EFS-Web and is hereby incorporated by reference in its entirety. The ASCII copy is named 106546-703050_PCT-Sequence-Listing_ST25.txt and is 47 kilobytes in size.

### FIELD

The present disclosure provides compositions comprising and methods for producing formative pluripotent stem cells (PSCs), and in vitro culture systems comprising such.

### BACKGROUND

Pluripotency, the ability of a single cell to generate all lineages of an adult organism, exists as an orderly continuum during a brief window of early development. Two states flanking the pluripotency continuum, naïve and primed, have been captured in vitro as embryonic stem cells (ESCs) and epiblast stem cells (EpiSCs), respectively. However, there is lack of stable pluripotent stem cells (PSCs) to model intervals between naïve and primed pluripotency.

### SUMMARY

The present disclosure is based, at least in part, on the surprising discovery that culture conditions that simultaneously activated FGF, TGF-β/Smad, and WNT/β-Catenin signaling pathways enabled direct derivation of pluripotent stem cells (PSCs) that model an interval between naïve and primed pluripotency. Such PSCs may be referred to herein as “formative PSCs” or “intermediate PSCs.” It has also been demonstrated that formative PSCs produced according to methods described herein shared transcriptomic similarities with mouse embryonic day 5-6 (E5-6) epiblasts and retained high competence for direct primordial germ cell (PGC) induction in vitro and germline chimera formation in vivo.

Accordingly, provided herein are methods for producing formative pluripotent stem cells (PSCs), and in vitro culture systems comprising such. The formative PSCs thus produced can be differentiated into cells of any type (e.g., cardiomyocytes or neurons).

The present disclosure provides, in some embodiments, a method for producing formative embryonic stem cells (ESCs), the method comprising (i) obtaining a population of reproductive cells, and (ii) culturing the population of reproductive cells in a medium that comprises a fibroblast growth factor (FGF) activator, a transforming growth factor beta (TGF-β) activator, and a WNT activator, thereby producing formative ESCs.

In some embodiments, the population of reproductive cells comprises blastocysts. In some embodiments, the population of reproductive cells are obtained from a subject. In some embodiments, the subject is selected from the group consisting of a human, a rodent, and an ungulate.

In some embodiments, the FGF activator is selected from the group consisting of a protein, a nucleic acid, a small molecule, and a combination thereof. In some embodiments, the FGF activator is FGF protein.

In some embodiments, the TGF-β activator is selected from the group consisting of a protein, a nucleic acid, a small molecule, and a combination thereof. In some embodiments, the TGF-β activator is TGF-β protein. In some embodiments, the TGF-β activator is Activin-A protein.

In some embodiments, the WNT activator is selected from the group consisting of a protein, a nucleic acid, a small molecule, and a combination thereof. In some embodiments, the WNT activator is WNT protein. In some embodiments, the WNT activator is a GSK3 inhibitor and/or a MEK inhibitor. In some embodiments, the GSK3 inhibitor is CHIR99021.

In some embodiments, step (ii) is performed for 1 to 8 days. In some embodiments, prior to step (ii), the population of reproductive cells is cultured in a medium that comprises at least one additional factor.

In some embodiments, the medium in step (ii) further comprises at least one additional factor. In some embodiments, the at least one additional factor is a MEK inhibitor, fetal bovine serum (FBS), leukemia inhibitory factor (LIF), a INK inhibitor, or a combination thereof. In some embodiments, the MEK inhibitor is PD0325901. In some embodiments, the INK inhibitor is SP600125.

In some embodiments, the formative ESCs produced in step (ii) are cultured in the presence of at least one differentiation factor. In some embodiments, the at least one differentiation factor is selected from the group consisting of a growth factor, a WNT inhibitor, a INK inhibitor and a TGF-β inhibitor. In some embodiments, the growth factor is fibroblast growth factor (FGF), Noggin, or a combination thereof. In some embodiments, the WNT inhibitor is IWP2. In some embodiments, the TGF-β inhibitor is SB431542. In some embodiments, the INK inhibitor is SP600125.

The present disclosure provides, in some embodiments, a method for producing formative induced pluripotent stem cells (iPSCs), the method comprising (i) obtaining a population of somatic cells, (ii) culturing the population of somatic cells in a first medium that comprises a reprogramming agent that modulates expression of at least one reprogramming gene selected from the group consisting of OCT3/4, p53, SOX2, KLF4, L-MYC, and LIN28, and (ii) culturing the population of somatic cells in a second medium that comprises a fibroblast growth factor (FGF) activator, a transforming growth factor beta (TGF-β) activator, and a WNT activator, thereby producing formative iPSCs.

In some embodiments, the population of somatic cells comprises embryonic fibroblasts. In some embodiments, the population of somatic cells are obtained from a subject. In some embodiments, the subject is selected from the group consisting of a primate, a rodent, and an ungulate.

In some embodiments, the reprogramming agent comprises a nucleic acid. In some embodiments, the nucleic acid encodes the reprogramming gene. In some embodiments, the nucleic acid encodes an interfering RNA that targets the reprogramming gene. In some embodiments, the nucleic acid is comprised in a vector. In some embodiments, the vector is an episomal vector.

In some embodiments, the FGF activator is selected from the group consisting of a protein, a nucleic acid, a small molecule, and a combination thereof. In some embodiments, the FGF activator is FGF protein.

In some embodiments, the TGF-β activator is selected from the group consisting of a protein, a nucleic acid, a small molecule, and a combination thereof. In some embodiments, the TGF-β activator is TGF-β protein. In some embodiments, the TGF-β activator is Activin-A protein.

In some embodiments, the WNT activator is selected from the group consisting of a protein, a nucleic acid, a small molecule, and a combination thereof. In some embodiments, the WNT activator is WNT protein. In some embodiments, the WNT activator is a GSK3 inhibitor. In some embodiments, the GSK3 inhibitor is CHIR99021.

In some embodiments, step (ii) is performed for 1 to 6 days. In some embodiments, step (iii) is performed for 1 to 8 days.

In some embodiments, the formative iPSCs produced in step (iii) are cultured in the presence of at least one differentiation factor.

In some embodiments, the at least one differentiation factor is selected from the group consisting of a growth factor, a WNT inhibitor, a JNK inhibitor, and a TGF-β inhibitor. In some embodiments, the growth factor is fibroblast growth factor (FGF), Noggin, or a combination thereof. In some embodiments, the WNT inhibitor is IWP2. In some embodiments, the TGF-β inhibitor is SB431542. In some embodiments, the JNK inhibitor is SP600125.

The present disclosure provides, in some embodiments, an in vitro culture system comprising (a) a population of cells, and (b) a medium that comprises a fibroblast growth factor (FGF) activator, a transforming growth factor beta (TGF-β) activator, and a WNT activator.

In some embodiments, the in vitro culture system further comprises a reprogramming agent that modulates expression of at least one reprogramming gene selected from the group consisting of OCT3/4, p53, SOX2, KLF4, L-MYC, and LIN28.

In some embodiments, the population of cells comprises reproductive cells or somatic cells. In some embodiments, the population of cells comprises blastocysts or embryonic fibroblasts.

In some embodiments, the population of reproductive cells are obtained from a subject. In some embodiments, the subject is selected from the group consisting of a primate, a rodent, and an ungulate.

In some embodiments, the FGF activator is selected from the group consisting of a protein, a nucleic acid, a small molecule, and a combination thereof. In some embodiments, the FGF activator is FGF protein.

In some embodiments, the TGF-β activator is selected from the group consisting of a protein, a nucleic acid, a small molecule, and a combination thereof. In some embodiments, the TGF-β activator is TGF-β protein. In some embodiments, the TGF-β activator is Activin-A protein.

In some embodiments, the WNT activator is selected from the group consisting of a protein, a nucleic acid, a small molecule, and a combination thereof. In some embodiments, the WNT activator is WNT protein. In some embodiments, the WNT activator is a GSK3 inhibitor. In some embodiments, the GSK3 inhibitor is CHIR99021.

In some embodiments, the medium further comprises at least one additional factor. In some embodiments, the at least one additional factor is a MEK inhibitor, fetal bovine serum (FBS), leukemia inhibitory factor (LIF), a INK inhibitor, or a combination thereof. In some embodiments, the MEK inhibitor is PD0325901. In some embodiments, the JNK inhibitor is SP600125.

In some embodiments, the medium further comprises at least one differentiation factor. In some embodiments, the at least one differentiation factor is selected from the group consisting of a growth factor, a WNT inhibitor, and a TGF-β inhibitor. In some embodiments, the growth factor is fibroblast growth factor (FGF), Noggin, or a combination thereof. In some embodiments, the WNT inhibitor is IWP2. In some embodiments, the TGF-β inhibitor is SB431542.

The present disclosure provides, in some embodiments, a method for producing formative embryonic stem cells (ESCs), the method comprising (i) obtaining a population of reproductive cells from a mouse, (ii) culturing the population of reproductive cells in a first medium that comprises a MEK inhibitor, and (iii) culturing the population of reproductive cells in a second medium that comprises a fibroblast growth factor (FGF) activator, a transforming growth factor beta (TGF-β) activator, and a WNT activator, thereby producing formative mouse ESCs.

The present disclosure provides, in some embodiments, a method for producing formative embryonic stem cells (ESCs), the method comprising (i) obtaining a population of reproductive cells from a horse, (ii) culturing the population of reproductive cells first in a medium that comprises fetal bovine serum (FBS), a fibroblast growth factor (FGF) activator, a transforming growth factor beta (TGF-β) activator, and a WNT activator, and (iii) culturing the population of reproductive cells in a second medium that comprises the fibroblast growth factor(FGF) activator, the transforming growth factor beta (TGF-β) activator, and the WNT activator, thereby producing formative horse ESCs.

The present disclosure provides, in some embodiments, a method for producing formative embryonic stem cells (ESCs), the method comprising (i) obtaining a population of reproductive cells from a pig, (ii) culturing the population of reproductive cells first in a medium that comprises fetal bovine serum (FBS), a fibroblast growth factor (FGF) activator, a leukemia inhibitory factor (LIF) activator, a transforming growth factor beta (TGF-β) activator, and a WNT activator, and (iii) culturing the population of reproductive cells in a second medium that comprises the fibroblast growth factor (FGF) activator, the leukemia inhibitory factor (LIF) activator, the transforming growth factor beta (TGF-β) activator, and the WNT activator, thereby producing formative pig ESCs.

The present disclosure provides, in some embodiments, a method for producing formative induced pluripotent stem cells (iPSCs), the method comprising: (i) obtaining a population of somatic cells from a human, (ii) culturing the population of somatic cells in a first medium that comprises a reprogramming agent that modulates expression of at least one reprogramming gene selected from the group consisting of OCT3/4, p53, SOX2, KLF4, L-MYC, and LIN28, and (iii) culturing the population of somatic cells in a second medium that comprises a fibroblast growth factor (FGF) activator, a transforming growth factor beta (TGF-β) activator, a INK inhibitor and a WNT activator, thereby producing formative human iPSCs.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1N include data showing derivation and characterization of mouse FTW-ESCs. FIG. 1A includes a schematic image of FTW-ESCs derivation from mouse blastocysts. FIG. 1B includes representative phase contrast images showing typical colony morphology of FTW-ESCs derived with CHIR99021 (left, passage 40) and 500 ng/mL Wnt3a (right, passage 7). Scale bar, 100 um. FIG. 1C includes a graph showing a growth curve of FTW-ESCs (passage 20 to 30) (mean±SD, n=6, biological replicates). FIG. 1D includes a graph showing single cell clonal efficiency of FTW-ESCs (passage 20 to 30) with and without ROCK inhibitor Y27632 treatment (mean±SD, n=3, biological replicates, N.S., not significant). FIG. 1E includes representative immunofluorescence (IF) images of FTW-ESCs at passage 16. Scale bar, 100 um. FIG. 1F includes slow cytometry results showing homogeneous expression of SOX2 and KLF4 in FTW-ESCs (passage 20 to 30) (mean±SD, n=3, biological replicates). FIG. 1G includes images showing Xist RNA FISH signals in female ESCs, FTW-ESCs and EpiSCs. Scale bars, 100 um. FIG. 1H includes a histogram showing increased percentages of cells with Xist FISH signal during FTW-ESCs to EpiSCs transition (mean±SD, n=3, biological replicates). FIG. 1I includes a graph showing flow cytometry analysis of X-EGFP FTW-ESCs. FIG. 1J includes images showing tight junction as visualized by ZO1 IF in ESCs, FTW-ESCs and EpiSCs. Scale bars, 100 um. FIG. 1K includes a graph showing relative expression levels of tight-junction related Claudin family genes in ESCs, FTW-ESCs and EpiSCs (mean±SD, n=4, biological replicates, *p<0.05, **p<0.01, ***p<0.001). FIG. 1L includes representative merged FL and BF images showing the expression pattern of Oct4-DE-EGFP in ESCs, FTW-ESCs and EpiSCs. Scale bars, 100 um. FIG. 1M includes a graph of flow cytometry results showing Oct4-DE-EGFP signal intensities and cell counts in ESCs, FTW-ESCs and EpiSCs. FIG. 1N includes a PCA plot of RNA-seq data from ESCs, FTW-ESCs and EpiSCs. See also FIG. 8 and FIG. 9.

FIGS. 2A-2G include data showing that FTW-ESCs share features characteristic of formative pluripotency but are distinct from EpiLCs. FIG. 2A includes a PCA plot of RNA-seq data from in vitro (ESCs, ASCs, RSCs, EPS-Deng, EPS-Liu, FTW-ESCs, EpiLCs and EpiSCs) and in vivo (E2.5 blastomeres, E3.5 and E4.5 ICMs, and E5.5-E7.5 epiblasts) samples. FIG. 2B includes PCA plots of the H3K4me3 and H3K27me3 ChIP-seq data from ESCs, FTW-ESCs, EpiSCs and 48 h EpiLCs. FIG. 2C includes a MA plot showing mean FPKM value against fold change per gene in FTW-ESCs versus ESCs (ESCs.Bao). Gene symbols are shown for selected naïve and formative genes listed below. FIG. 2D includes the IGV Genome Browser view showing H3K4me3 and H3K27me3 modifications of representative formative and naïve genes in ESCs and FTW-ESCs. FIG. 2E includes a VENN diagram (left) showing the number of commonly and differentially expressed transcription factors and co-factors among ESCs, FTW-ESCs and EpiSCs (compared with MEFs, FC >2 and P<0.05), and a graph (right) of top 8 enriched GO terms of 79 TFs and cofactors specifically upregulated in FTW-ESCs. FIG. 2F includes representative FL images of GPI-EGFP labelled ESCs, FTW-ESCs and EpiSCs cultured in 3D Matrigel condition at day 2. Scale bars, 100 um. FIG. 2G includes a graph showing ratio of different types of structures (disorganized, rosette and lumen) at day 1 and day 2 shown in FIG. 2F. See also FIG. 10.

FIGS. 3A-3K include data showing that FTW-ESCs exhibit chimera and PGC dual-competency. FIG. 3A includes representative IF images showing chimeric contribution and differentiation of FTW-ESCs. EGFP, FTW-ESCs derivatives stained with a GFP antibody; Lineage markers, lineage markers: NESTIN, ectoderm; cTNT, mesoderm; and FOXA2, endoderm. Merged images (with DAPI) and higher magnification images of boxed regions were shown on the right. Scale bar, 100 mm. FIG. 3B includes an image showing Coat-color chimeras (yellow arrows) generated by injecting C57BL/6 FTW-ESCs into B6-albino (B6(Cg)-Tyrc-2J/J host blastocysts. FIG. 3C includes a graph showing ratio of Coat-color chimeras generated form ESCs and FTW-ESCs at passage 5, total numbers of live pups (n) were showed in the chart. FIG. 3D includes an image showing germline-transmission of FTW-ESCs derived chimera. Female: WT, B6-albino. Male: B6 (FTW-ESCs)/B6-albino chimera. FIG. 3E includes representative FL images showing day 2, day 4 and day 6 PGC-LCs (visualized by Blimp1-mVenus (BV) and Stella-ECFP (SC) expression) induced from FTW-ESCs with a FGFR inhibitor (PD173074). Scale bar, 100 um. FIG. 3F includes data showing FACS analysis of BV/SC expression after 2, 4 and 6 days of PGC-LC induction of FTW-ESCs. FIG. 3G includes data showing gene expression dynamics during PGC-LC induction from FTW-ESCs. (mean±SD, n=3). Black lines: BV and SC double positive cells, Gray dashed lines: BV and SC double negative cells. FIG. 3H includes representative IF (TFAP2C and BLIMP1) images of PGC-LCs induced from FTW-ESCs. Insets are higher magnification images of boxed regions. Scale bar, 100 um. FIG. 3I includes representative IF images of 5mC after 6 days of PGC-LC induction of FTW-ESCs. Dotted lines delineate cells with reduced 5mC signal (PGC-LCs are labeled by BLIMP1 staining). Scale bar, 100 um. FIGS. 3J-3K include representative IF images of H3K9me2 (FIG. 3J) and H3K27me3 (FIG. 3K) after 6 days of PGC-LC induction of FTW-ESCs. Insets are higher magnification images of boxed regions. Scale bar, 100 um. See also FIG. 11.

FIG. 4A-4M include data showing derivation and characterization of horse FTW-PSCs. FIG. 4A includes a schematic of FTW-eqPSCs derivation from equine blastocysts and embryonic fibroblasts. FIG. 4B includes representative BF images of equine blastocyst, and FTW-eqESCs (passage 23) grown on MEF feeders and feeder-free condition (Matrigel, MG). Scale bars, 100 μm. FIG. 4C includes representative BF images of equine embryonic fibroblasts, and FTW-eqiPSCs (passage 38) grown on MEF feeders and feeder-free condition (MG). Scale bars, 100 μm. FIG. 4D includes data showing genomic PCR with plasmid-specific primers showing all the exogenous reprogramming factors were silenced after 20 passages in two independent FTW-eqiPSC lines. FIG. 4E includes representative IF images showing FTW-eqESCs (passage 19) expressed pluripotency markers (SOX2 and OCT4) but not extraembryonic lineage markers (TE, EMOES; PE, GATA6). Scale bars, 100 μm. FIG. 4F includes flow cytometry results indicating majority of FTW-eqiPSCs expressed OCT4 and SOX2. FIG. 4G includes images showing alkaline phosphatase (AP) staining of FTW-eqESCs (top) and FTW-eqiPSCs (bottom). Scale bars, 100 μm. FIG. 4H includes a graph showing growth curves of FTW-eqPSCs (two FTW-eqESC lines (passage 10-20) and two FTW-eqiPSC lines (passage 2030)). FIG. 4I includes a graph showing single cell clonal efficiencies of FTW-eqPSCs (two FTW-eqESC lines and two FTW-eqiPSC lines) with and without ROCK inhibitor Y27632 treatment (mean±SD, n=3, biological replicates, N.S.=no significant). FIG. 4J includes a graph showing hierarchical clustering and correlation analysis of FTW-eqiPSCs, FTW-eqESCs and in vivo equine samples (Eq-ICM, Eq-EEF and Eq-TE). Expression levels (FPKMs) of all detected genes were used to draw this plot. FIG. 4K includes graphs showing RNA-seq, and H3K4me3 and H3K27me3 tracks of selected pluripotency genes in FTW-eqPSCs. FIG. 4L includes representative IF images showing embryoid body (EB) differentiation of FTW-eqiPSCs into ectoderm (TUJ1), mesoderm (cTnT), and endoderm (AFP) lineages. Scale bars, 100 μm. FIG. 4M includes representative H&E staining images showing teratomas formed by FTW-eqiPSCs contained tissues from all three embryonic germ layers. See also FIG. 12.

FIGS. 5A-5I include data showing that FTW-eqPSCs harbor mouse formative features and are chimera and PGC dual-competent. FIG. 5A includes representative BF and IF images showing mKO-labeled FTW-eqiPSCs engrafted equine ICM and expressed OCT4 following 2 days in vitro (2 DIV) embryo culture. A higher magnification image of the boxed area was shown on the right. Scale bar, 100 um. FIG. 5B includes representative FL images showing ICM incorporation of mKO-labeled FTW-eqiPSCs in mouse, sheep, goat, and pig blastocysts (BL). Red, mKO-labeled FTW-eqiPSCs. FIG. 5C includes representative images showing interspecies chimera contribution of EGFP-labeled FTW-eqESCs to an E9.5 mouse embryo (Chimera #1). Left, WT control. Right, horse-mouse chimeric embryo. FIG. 5D includes representative immunofluorescence images showing interspecies chimera contribution and differentiation of FTW-eqESCs (Chimera #1). EGFP, FTW-eqESCs derivatives stained with a GFP antibody; Lineage markers: TuJ1, ectoderm; SMA, mesoderm; and FOXA2, endoderm. Merged images (with DAPI) and higher magnification images of boxed regions were shown on the right. Scale bar, 100 mm. FIG. 5E includes a PCA plot of RNA-seq data from mouse (mESCs, XPSCs, mEpiLCs and mEpiSCs) and equine (Eq-ICM, FTW-eqESCs and FTW-eqiPSCs) samples. FIG. 5F includes a graph showing direct induction of PGC-LCs from FTW-eqESCs. qRT-PCR results showing upregulation of PGC-LCs markers in FTW-eqESCs after 3 days induction (mean±SD, n=3, biological replicates, *p<0.05, **p<0.01, ***p<0.001). FIG. 5G includes representative IF images of TFAP2C, PRDM1 and DPPA3 after 3 days PGC-LC induction from FTW-eqESCs. Scale bar, 100 um. FIG. 5H includes a schematic of the strategy of the generation of Nanos3-EGFP-KI reporter FTW-ESC lines (top), and representative images showing day 1, day 2 and day 3 PGC-LCs induction from Nanos3-EGFP-KI FTW-eqESCs (bottom). FIG. 5I includes data from FACS analysis of EGFP expression in un-induced, and 1, 2 and 3 days after PGC-LC induction from Nanos3-EGFP-KI FTW-eqESCs. See also FIG. 13.

FIGS. 6A-6J includes data showing derivation and characterization of human FTW-iPSCs. FIG. 6A includes a schematic of FTW-hiPSCs generation from human fibroblasts. FIG. 6B includes a representative BF image showing the typical colony morphology of FTW-hiPSCs (passage 31). Scale bars, 100 um. FIG. 6C includes representative IF images showing FTW-hiPSCs (passage 26) expressed core pluripotency markers (SOX2 and OCT4) and formative pluripotency marker OTX2. Scale bars, 100 um. FIG. 6D includes representative IF images showing the naïve (SUSD2 and KLF17) and primed (CD24) pluripotency markers were not expressed in FTW-hiPSCs. Naïve hESCs were cultured in the 5iLA condition. Primed hiPSCs were cultured in NBFR condition. Scale bar, 100 um. FIG. 6E includes flow cytometry result showing the majority of FTW-hiPSCs expressed OTX2 but not CD24. FIG. 6F includes a histogram showing the doubling time of FTW-hiPSCs and primed hiPSCs (NBFR condition) at passage 20-30 (mean±SD, n=3, biological replicates, *p<0.05). FIG. 6G includes a graph of single cell clonal efficiencies of FTW-hiPSCs and primed hiPSCs (NBFR condition) with and without ROCK inhibitor Y27632 treatment (mean±SD, n=3, biological replicates, ***p<0.001, N.S.=no significant). FIG. 6H includes a PCA plot of RNA-seq data from naïve hESCs (5iLA and Reset condition), FTW-hiPSCs and primed hESCs. FIG. 6I includes a heatmap analysis showing 936 genes specifically up-regulated in FTW-hiPSCs compared to naïve and primed hESCs. FIG. 6J includes data showing GO analysis of 936 FTW-hiPSCs specific genes in FIG. 6I. See also FIG. 14.

FIGS. 7A-7F include data showing that FTW-eqPSCs harbor mouse formative features and are PGC and chimera dual-competent. FIG. 7A includes a PCA plot of RNA-seq data from in vitro (Reset, HNES, 5/6iLA, 4i, FTW-hiPSCs, 2iLI, NHSM, 2iLIF and primed hESCs) and in vivo (human E6, E8, E10 and E12 epiblasts) samples. FIG. 7B includes a MA plot showing mean FPKM value against fold change per gene in FTW-hiPSCs versus naïve hESCs (Reset and Gene symbols are shown for selected naïve (red) and formative (green) genes. FIG. 7C includes data showing FACS analysis of two human PGC-related surface markers (Ep-CAM and CD49f) expression after 2, 4 and 6 days of PGC-LC induction of FTW-hiPSCs. FIG. 7D includes data showing gene expression dynamics during PGC-LC induction from FTW-hiPSCs. (mean±SD, n=3). Black lines: Ep-CAM and CD49f double positive cells, Gray dash lines: Ep-CAM and CD49f double negative cells. FIG. 7E includes representative IF (TFAP2C, PRDM1 and NANOS3) images of PGC-LCs induced from FTW-hiPSCs. Scale bar, 100 um. FIG. 7F includes a summary figure of formative-like XPSCs. See also FIG. 14.

FIGS. 8A-8I include data showing derivation and characterization of mouse FTW-ESCs. FIG. 8A includes a schematic of the method and representative BF images showing mouse FTW-ESCs derivation from E3.5 blastocysts. Scale bars, 100 um. FIG. 8B includes data showing a karyotype analysis of female (Left) and male (Right) FTW-ESCs lines at passage 16.

FIG. 8C includes data showing relative expression levels of core, naïve and primed pluripotency genes in ESCs, FTW-ESCs and EpiSCs (mean±SD, n=3, biological replicates, *p<0.05, **p<***p<0.001). FIG. 8D includes a histogram showing TOPFlash and FOPFlash reporter activity in ESCs, FTW-ESCs and EpiSCs. (mean±SD, n=4, biological replicates). FIG. 8E includes data showing analysis of FGF/Erk and TGF-β/Smad pathway activation status in ESCs (cultured in N2B27 medium supplied with CHIR99021, PD0325901 and LIF, N2B27-2iL), FTW-ESCs (cultured in N2B27 medium supplied with FGF2, Activin-A and CHIR99021, N2B27-FAC) and EpiSCs (cultured in N2B27 medium supplied with FGF2 and IWR1, N2B27-FR). Representative western blots showing phospho-MAPK and Smad2/3 (P-MAPK and P-Smad2/3) and the total protein levels of MAPK and Smad2/3. Vinculin was used as a loading control. FIG. 8F includes a heatmap of qRT-PCR results showing the expression pattern of FTW-ESCs cultured in different FTW media after 2 passages. WNT3a-L (50 ng/ml); WNT3a-H (500 ng/ml). FIG. 8G includes representative merged IF images of FTW-ESCs cultured in different FTW media after 2 passages. Scale bar, 50 um. FIG. 8H includes data showing ratio of OCT4 and SOX2 positive (or negative) cells showing in FIG. 8G. FIG. 8I includes qRTPCR results of core pluripotency (Pou5f1 and Sox2), naïve (Nanog and Klf4), primed (0tx2 and Dnmt3b) and lineage markers (Sox1, Gata4 and Gata6) in FTW-ESCs cultured in FT, FW, TW and FTW media at each passage from passage 1 (P1) to passage 4 (P4) (n=3, biological replicates).

FIGS. 9A-9G include data showing that mouse FTW-ESCs share features of both Naïve and Primed PSCs. FIG. 9A includes images showing FTW-ESCs and EpiSCs can be successively converted from ESCs by culture adaptations. Scale bars, 100 um. FIG. 9B includes representative images of Xist RNA FISH staining showing the number of cells with positive Xist signal increased during FTW-ESCs to EpiSCs conversion. Scale bars, 100 um. FIG. 9C includes representative FL and BF images showing the number of cells with positive X-EGFP signal decreased during FTW-ESCs to EpiSCs conversion. Scale bars, 100 um. FIG. 9D includes a graph of FACS analysis of X-EGFP signal in FIG. 9C. FIG. 9E includes a graph of ratio of Oct4-DE-EGFP positive colonies in ESCs, FTW-ESCs and EpiSCs showing in FIG. 2E. FIG. 2F includes a heatmap showing differential expression of 296 pluripotency related genes in ESCs, FTW-ESCs and EpiSCs. FIG. 9G includes data showing H3K4me3 and H3K27me3 ChIP-seq signals in ESCs, FTW-ESCs and EpiSCs.

FIGS. 10A-10F include data showing that FTW-ESCs share features characteristic of formative pluripotency. FIG. 10A includes data showing GO analysis of 2783 H3K4me3-high genes in FTW-ESCs. FIG. 10B includes data showing GO analysis of 2215 H3K27me3-high genes in FTW-ESCs. FIG. 10C includes a heat map of fold change of genes (compared to ESCs) for mitochondrial complex COX and enzymes involved in glycolysis and the tricarboxylic acid cycle selected from the RNA-seq data set. FIG. 10D includes a MA plot showing mean FPKM value against fold change per gene in FTW-ESCs versus EpiLCs (48 h). Gene symbols are shown for selected naïve, formative and primed genes. FIG. 10E includes the IGV Genome Browser view showing H3K4me3 and H3K27me3 tracks of representative naïve and formative genes in FTW-ESCs and EpiLCs (48 h). FIG. 10F includes images of western blots showing the expression of NANOG, OTX2, OCT4 and GAPDH (loading control) proteins in FTW-ESCs and EpiLCs (48 h).

FIGS. 11A-11E include data showing that FTW-ESCs exhibit chimera and PGC dual-competency. FIG. 11A includes representative IF images of linage markers in chimeras generated from FTW-ESCs. FTW-ESCs derivatives stained with a GFP antibody; lineage markers: TuJ and PAX6, ectoderm; SMA and T, mesoderm; and AFP and SOX17, endoderm. Merged images (with DAPI) and higher magnification images of boxed regions were shown on the right. Scale bar, 100 mm. FIG. 11B includes representative images showing chimeric contribution of Oct4-DE-EGFP FTW-ESCs to E13.5 mouse gonads. G: Gonads, M: Mesonephros. FIG. 11C includes representative FL images showing day 2, 4 and 6 PGC-LCs induction of FTW-ESCs, which is visualized by Blimp1-mVenus (BV) and Stella-ECFP (SC) expression. Scale bar, 100 um. FIG. 11D includes representative images (top) and histogram (bottom) showing the ratio of BVSC positive and/or negative cells after 4 days of PGC-LC induction with the treatment of different pathway inhibitors. FIG. 11E includes a histogram showing PGC-LC induction efficiencies from de novo derived FTW-ESCs with or without FGF inhibitor PD173074.

FIGS. 12A-12O includes data showing derivation and characterization of horse FTW-PSCs. FIGS. 12A-12B include a schematic of FTW-eqESCs (FIG. 12A) and FTW-eqiPSCs (FIG. 12B) derivation. FIG. 12C includes data showing that the EGFP episomal vector was gradually silenced after long term passages (P20). FTW-eqiPSCs were imaged for EGFP expression and analyzed by flow cytometry at passage 5, 10, 15 and 20. FIG. 12D includes a graph showing total copy numbers of all four episomal vectors in two FTW-eqiPSCs lines at indicated passages. FIG. 12E includes a graph showing doubling time of two FTW-eqiPSC lines before (<passage15) and after (>passage20) the exogenous reprogramming factors were silenced (mean±SD, n=7, biological replicates). FIG. 12F includes data from cell cycle analysis of two FTW-eqiPSC lines before (at passage5) and after (at passage20) the exogenous reprogramming factors were silenced. FIG. 12G includes data from RT-PCR with equine-specific primers showing the equine endogenous pluripotency genes were expressed in both FTW-eqESCs and FTW-eqiPSCs.

FIG. 12H includes data from a chromosome analysis of FTW-eqPSCs (two FTW-eqESC lines and two FTWeqiPSClines). FIGS. 12I-12J include a Heatmap (FIG. 12I) and GO analysis (FIG. 12J) of 1798 commonly up-regulated genes in eqESCs, FTW-eqiPSCs and eq-ICMs. FIG. 12K includes data from global H3K4me3 and H3K27me3 ChIP-seq signals in FTWeqESCs and FTW-eqiPSCs. FIG. 12L includes a schematic of the experimental plan for directed cardiomyocytes differentiation from FTW-eqiPSCs, and a representative BF image (Right) of FTW-eqiPSCs derived cardiomyocytes. Scale bar, 100 um. FIG. 12M includes representative IF images of FTW-eqiPSCs derived cardiomyocytes. Scale bars, 100 um. FIG. 12N includes a schematic of the experimental plan for directed neurons differentiation from FTW-eqiPSCs, and a representative BF image (Right) of FTW-eqiPSCs derived neurons. Scale bar, 100 um. FIG. 12O includes representative IF images of FTW-eqiPSCs derived neuronal lineages. Scale bars, 100 um.

FIGS. 13A-13L includes data showing that FTW-eqPSCs harbor mouse formative features and are chimera and PGC dualcompetent. FIG. 13A includes representative IF images showing mKO-labeled FTW-eqiPSCs engrafted into mouse, pig, and sheep ICM and expressed OCT4 (mouse) or SOX2 (pig and sheep) following 3 days in vitro embryo culture (3 DIV). Scale bar, 100 um. FIG. 13B includes representative BF and IF images showing mKOlabeled FTW-eqiPSCs engrafted into post-implantation mouse epiblast and expressed OCT4 following 6 days in vitro (6 DIV) embryo culture. Higher magnification images of the boxed area were shown at the bottom. FIG. 13C includes representative images showing interspecies chimera contribution of EGFP-labeled FTW-eqESCs to an E7.75 mouse embryo. Left, WT control. Right, horse-mouse chimeric embryo. FIG. 13D includes representative images showing interspecies chimera contribution of EGFPlabeled FTW-eqESCs to an E9.5 mouse embryo (Chimera #2). Left, WT control. Right, horsemouse chimeric embryo. FIG. 13E includes representative immunofluorescence images showing interspecies chimera contribution and differentiation of FTW-eqESCs (Chimera #2). FTW-eqESCs derivatives stained with a GFP antibody; lineage markers: TuJ1, ectoderm; SMA, mesoderm; and FOXA2, endoderm. Merged images (with DAPI) and higher magnification images of boxed regions were shown on the right. Scale bar, 100 mm.

FIG. 13F includes a heatmap showing fold changes of 1,619 primed pluripotency related genes in mouse (ESCs, EpiLCs and EpiSCs) and equine (Eq-ICM, FTW-eqESCs and FTW-eqiPSCs) samples. Black and grey represent log 2-transformed fold changes <0 and >0, respectively. FIG. 13G includes images of RT-PCR results with equine-specific primers showing several mouse formative pluripotency genes were expressed in both FTW-eqESCs and FTWeqiPSCs. FIG. 13H includes the IGV Genome Browser view showing gene expression levels, H3K4me3 and H3K27me3 modifications of selected formative pluripotency related genes. FIG. 13I includes representative IF (Phalloidin) images of FTW-eqESCs cultured in 3D Matrigel condition at day 2. Scale bars, 100 um. FIG. 13J includes representative images of tight junction as visualized by ZO1 immunostaining in FTW-eqESCs. Scale bars, 100 um. FIG. 13K includes a schematic of the strategy of Nanos3-EGFP-KI reporter FTW-eqESC lines generation. FIG. 13L includes a histogram showing day 4 PGC-LC induction efficiencies in the presence of different pathway inhibitors.

FIGS. 14A-14G include data showing characterization of human FTW-iPSCs and cross-species comparison. FIG. 14A includes a graph showing total copy numbers of episomal vectors in FTW-hiPSCs_#2 at indicated passages (P12, P25 and P33). The ENBA (Epstein-Barr Nuclear Antigen-1) sequence were used to detect episomal vectors. FIG. 14B includes representative H&E staining images showing teratomas formed by FTW-hiPSCs_#2 (passage 30) contained tissues from all three embryonic germ layers. FIG. 14C includes representative images showing tight junction as visualized by ZO1 immunostaining in FTW-hiPSCs. Scale bars, 100 um. FIG. 14D includes representative IF (Phalloidin) images of FTW-hiPSCs cultured in 3D Matrigel condition at day 2. Scale bars, 100 um. FIG. 14E includes a graph showing human PGC-LC induction efficiency from human FTW-iPSCs in the presence of different pathway inhibitors (FGF, TGFβ, WNT or LIF inhibitors). FIG. 14F includes data from hierarchical clustering and correlation analysis of FTW-mESCs, FTW-eqESCs/iPSCs and FTW-hiPSCs. Expression levels (FPKMs) of all detected genes were used to draw this plot. FIG. 14G includes a heatmap and GO analysis of species-specific genes of human (n=1220), equine (n=1269) and mouse XPSCs (n=1540).

FIG. 15A-C include data showing differentiation and growth of human FTW-iPSCs in the presence of SP600125 (a INK inhibitor). FIG. 15A includes a representative brightfield image of human FTW-iPSCs supplied with SP600125 (a INK inhibitor). FIG. 15B includes a graph showing doubling time in the presence of absence of different concentrations of SP600125 (a JNK inhibitor). FIG. 15C includes representative immunofluorescence images showing human FTW-iPSCs supplied with SP600125 expressed pluripotency markers (SOX2 and OCT4). Scale bars, 100 nm. FIGS. 15D-E shows data showing differentiated white rhino FTWi-iPSCs generated according to the methods of the present disclosure. FIG. 15D includes a representative brightfield image of Rhino FTW-iPSCs. Scale bars, 100 nm. FIG. 15E includes representative immunofluorescence images showing Rhino FTW-iPSCs expressed pluripotency markers (SOX2 and OCT4). Scale bars, 100 nm. FIGS. 15F-15G shows differentiated pig FTW-PSCs prepared according to various methods of the present disclosure. FIG. 15F includes representative brightfield images of Pig FTW-ESCs. Scale bars, 100 nm. FIG. 15G includes representative immunofluorescence images showing Pig FTW-ESCs expressed pluripotency markers (SOX2 and OCT4). Scale bars, 100 nm.

The details of one or more embodiments of the invention are set forth in the description below. Other features or advantages of the present invention will be apparent from the following drawings and detailed description of several embodiments, and also from the appended claims.

### DETAILED DESCRIPTION

Dynamic pluripotent stem cell (PSC) states represent in vitro adaptations of the in vivo pluripotency continuum. Two states flanking the pluripotency continuum, naïve and primed, have been captured in vitro as embryonic stem cells (ESCs) and epiblast stem cells (EpiSCs), respectively. “Naïve” mouse ESCs most closely resemble the naïve epiblast of a mature blastocyst (approximately embryonic day 4, ˜E4) while “primed” EpiSCs display a gene expression signature more similar to the anterior epiblast of a late-gastrula-stage embryo (approximately embryonic day 7, ˜E7). Pluripotent stem cells (PSCs) in intervals between naïve and primed pluripotency have yet to be captured, and therefore their transcriptional, epigenetic, and functional features are not well understood.

The present disclosure is based, at least in part, on the surprising discovery that culture conditions that simultaneously activated FGF, TGF-β/Smad, and WNT/β-Catenin signaling pathways enabled direct derivation of PSCs that model an interval between naïve and primed pluripotency. Such PSCs may be referred to herein as “formative PSCs” (also known as “intermediate PSCs”). Formative PSCs produced according to methods described herein shared transcriptomic similarities with embryonic day 5-6 (E5-6) epiblasts and retained high competence for direct primordial germ cell (PGC) induction in vitro and germline chimera formation in vivo. No PSCs known in the art thus far have demonstrated such competency for direct PGC induction in vitro. It has also been demonstrated that methods described herein may be useful for producing formative PSCs for various species such as rodent (mouse), ungulate (horse), and primate (human) species.

Accordingly, provided herein are methods for producing formative pluripotent stem cells (PSCs), and in vitro culture systems comprising such. The formative PSCs thus produced can be differentiated into cells of any type (e.g., cardiomyocytes or neurons).

### I. Methods for Producing Formative Pluripotent Stem Cells (PSCs)

Combination of any of the FGF activators, TGF-β activators, and WNT activators, e.g., those described herein, can be used for producing formative pluripotent stem cells (PSCs) (e.g., embryonic stem cells (ESCs) or induced pluripotent stem cells (iPSCs)) in ex vivo or in vitro culture. Formative PSCs refers to PSCs that exist in the developmental continuum between the naïve and primed stages. As such, formative PSCs may possess molecular features distinct from their naïve and primed counterparts such as distinct molecular features (e.g., transcriptional and epigenetic features) and distinct functional features (e.g., dual competency for chimera formation and primordial germ cell (PGC) specification).

Thus, the present disclosure provides culturing methods (e.g., ex vivo culturing methods) for producing formative PSCs in cell cultures by culturing cells (e.g., blastocysts or embryonic fibroblasts) in the presence of at least one FGF activator, at least one TGF-β activator, and at least one WNT activator. The formative PSCs thus produced can be differentiated into cells of any type (e.g., cardiomyocytes or neurons).

To perform the culturing methods described herein, a suitable population of cells can be obtained from any suitable source. In some embodiments, the population of cells can be obtained from a subject, for example, from tissue (e.g., embryotic tissue), bone (e.g., bone marrow), blood (e.g., peripheral blood or umbilical cord blood), bodily fluid (e.g., tear, urine, or saliva), serum, plasma, or protein, from a subject via any means known in the art. A subject includes, but is not limited to, a human or a non-human mammal such as a rodent (e.g., a mouse or a rat) or an ungulate (e.g., a horse or a pig).

Any suitable population of cells can be used in methods for producing formative PSCs as described herein. In some embodiments, the population of cells comprises reproductive cells, e.g., female germline stem cells and progeny thereof. Examples of reproductive cells include, but are not limited to, embryos, oocytes, zygotes, blastomeres, morulae, and blastocysts.

In some embodiments, the population of cells comprises somatic cells such as fibroblasts (e.g., embryonic fibroblasts or skin fibroblasts). Somatic cells may be obtained by well-known methods from different organs, e.g., skin, lung, pancreas, liver, stomach, intestine, heart, reproductive organs, bladder, kidney, urethra, and other urinary organs. Examples of somatic cells include, but are not limited to, adult stem cells, Sertoli cells, endothelial cells, granulosa epithelial, neurons, pancreatic islet cells, epidermal cells, epithelial cells, hepatocytes, hair follicle cells, keratinocytes, hematopoietic cells, melanocytes, chondrocytes, lymphocytes (B lymphocytes and T lymphocytes), erythrocytes, macrophages, monocytes, mononuclear cells, fibroblasts, cardiac muscle cells, and other muscle cells.

Any of the populations of cells described herein can be cultured in a suitable medium (e.g., cell culture medium) in the presence of an effective amount at least one FGF activator, at least one TGF-β activator, and at least one WNT activator such as those described herein for a suitable period of time, e.g., at least 18 hours, at least about 24 hour, at least 36 hours, at least 48 hours, at least 60 hours, at least 72 hours, at least 84 hours, at least 96 hours, at least about 5 days, at least about 6 days, at least about 7 days, at least about 8 days, or longer.

An “effective amount,” “effective dose,” or an “amount effective to”, as used herein, refers to an amount of a FGF activator, a TGF-β activator, and a WNT activator described herein that is effective in providing at least one characteristic of formative PSCs (e.g., chimera competence, PGC specification responsiveness, and/or multi-lineage differentiation potential). Such characteristics may be monitored by conventional methods or may be monitored according to methods described herein. An effective amount may vary depending on, for example, the FGF activator, the TGF-β activator, and the WNT activator used.

For example, the effective amount of the FGF activator, the TGF-β activator, and the WNT activator for culturing the population of cells in methods described herein results in an increase in the proportion of cells in the formative stage of pluripotency by at least 10% or more, including, e.g., at least about 20%, at least about 30%, at least about 40%, at least about 50%, at least about 60%, at least about 70%, at least about 80%, at least about 90% or more, as compared to the proportion of cells in the formative stage of pluripotency when the population of cells is cultured without the FGF activator, the TGF-β activator, and the WNT activator.

In some embodiments, the effective amount of the FGF activator, the TGF-β activator, and the WNT activator for culturing the population of cells in methods described herein results in an increase in chimera formation efficiency in the population of cells by at least 10% or more, including, e.g., at least about 20%, at least about 30%, at least about 40%, at least about 50%, at least about 60%, at least about 70%, at least about 80%, at least about 90% or more, as compared to the chimera formation efficiency when the population of cells is cultured without the FGF activator, the TGF-β activator, and the WNT activator.

In some embodiments, the effective amount of the FGF activator, the TGF-β activator, and the WNT activator for culturing the population of cells in methods described herein results in an increase in primordial germ cell (PGC) induction efficiency in the population of cells by at least 10% or more, including, e.g., at least about 20%, at least about 30%, at least about 40%, at least about 50%, at least about 60%, at least about 70%, at least about 80%, at least about 90% or more, as compared to the PGC induction efficiency when the population of cells is cultured without the FGF activator, the TGF-β activator, and the WNT activator.

In some embodiments, the effective amount of the FGF activator, the TGF-β activator, and the WNT activator for culturing the population of cells in methods described herein results in increased expression of a formative pluripotency marker (e.g., those described herein such as OTX2) in the population of cells by at least 10% or more, including, e.g., at least about 20%, at least about 30%, at least about 40%, at least about 50%, at least about 60%, at least about 70%, at least about 80%, at least about 90% or more, as compared to expression of the formative pluripotency marker when the population of cells is cultured without the FGF activator, the TGF-β activator, and the WNT activator.

In some embodiments, the effective amount of the FGF activator, the TGF-β activator, and the WNT activator for culturing the population of cells in methods described herein results in increased expression of a core pluripotency marker (e.g., those described herein such as SOX2 and OCT4) in the population of cells by at least 10% or more, including, e.g., at least about 20%, at least about 30%, at least about 40%, at least about 50%, at least about 60%, at least about 70%, at least about 80%, at least about 90% or more, as compared to expression of the core pluripotency marker when the population of cells is cultured without the FGF activator, the TGF-β0 activator, and the WNT activator.

In some embodiments, the effective amount of the FGF activator, the TGF-β activator, and the WNT activator for culturing the population of cells in methods described herein results in reduced expression of a naïve pluripotency marker (e.g., those described herein such as SUSD2 and KLF17) in the population of cells by at least 10% or more, including, e.g., at least about 20%, at least about 30%, at least about 40%, at least about 50%, at least about 60%, at least about 70%, at least about 80%, at least about 90% or more, as compared to expression of the naïve pluripotency marker when the population of cells is cultured without the FGF activator, the TGF-β activator, and the WNT activator.

In some embodiments, the effective amount of the FGF activator, the TGF-β activator, and the WNT activator for culturing the population of cells in methods described herein results in reduced expression of a primed pluripotency marker (e.g., those described herein such as CD24) in the population of cells by at least 10% or more, including, e.g., at least about 20%, at least about 30%, at least about 40%, at least about 50%, at least about 60%, at least about 70%, at least about 80%, at least about 90% or more, as compared to expression of the primed pluripotency marker when the population of cells is cultured without the FGF activator, the TGF-β activator, and the WNT activator.

An effective amount of a FGF activator for the methods described herein can be between 0.1 and 10,000 ng/ml. In some embodiments, the effective amount of the FGF activator for the methods described herein can be between 1 and 10,000 ng/ml, between 10 and 10,000 ng/ml, between 100 and 10,000 ng/ml, between 1,000 and 10,000 ng/ml, between 5,000 and 10,000 ng/ml, between 0.1 and 5,000 ng/ml, between 1 and 5,000 ng/ml, between 10 and 5,000 ng/ml, between 1,000 and 5,000 ng/ml, or between 2,500 and 5,000 ng/ml.

In some embodiments, the effective amount of the FGF activator for the methods described herein can be between 0.1 and 100 μM. In some embodiments, the effective amount of the FGF activator for the methods described herein can be between 0.1 and 90 μM, between 0.1 and 80 μM, between 0.1 and 70 μM, between 0.1 and 60 μM, between 0.1 and 50 μM, between 0.1 and 40 μM, between 0.1 and 30 μM, between 0.1 and 20 μM, between 0.1 and μM, between 0.1 and 1 μM, and between 0.1 and 0.5 μM. In some embodiments, the effective amount of the FGF activator for the methods described herein can be between 0.5 and 100 μM, between 1 and 100 μM, between 10 and 100 μM, between 20 and 100 μM, between 30 and 100 μM, between 40 and 100 μM, between 50 and 100 μM, between 60 and 100 μM, between 70 and 100 μM, between 80 and 100 μM, and between 90 and 100 μM.

An effective amount of a TGF-β activator for the methods described herein can be between 0.1 and 10,000 ng/ml. In some embodiments, the effective amount of the TGF-β activator for the methods described herein can be between 1 and 10,000 ng/ml, between 10 and ng/ml, between 100 and 10,000 ng/ml, between 1,000 and 10,000 ng/ml, between 5,000 and 10,000 ng/ml, between 0.1 and 5,000 ng/ml, between 1 and 5,000 ng/ml, between 10 and 5,000 ng/ml, between 1,000 and 5,000 ng/ml, or between 2,500 and 5,000 ng/ml.

In some embodiments, the effective amount of the TGF-β activator for the methods described herein can be between 0.1 and 100 μM. In some embodiments, the effective amount of the TGF-β activator for the methods described herein can be between 0.1 and 90 μM, between 0.1 and 80 μM, between 0.1 and 70 μM, between 0.1 and 60 μM, between 0.1 and 50 μM, between 0.1 and 40 μM, between 0.1 and 30 μM, between 0.1 and 20 μM, between 0.1 and μM, between 0.1 and 1 μM, and between 0.1 and 0.5 μM. In some embodiments, the effective amount of the TGF-β activator for the methods described herein can be between 0.5 and 100 μM, between 1 and 100 μM, between 10 and 100 μM, between 20 and 100 μM, between 30 and 100 μM, between 40 and 100 μM, between 50 and 100 μM, between 60 and 100 μM, between 70 and 100 μM, between 80 and 100 μM, and between 90 and 100 μM.

An effective amount of a WNT activator for the methods described herein can be between 0.1 and 10,000 ng/ml. In some embodiments, the effective amount of the WNT activator for the methods described herein can be between 1 and 10,000 ng/ml, between 10 and ng/ml, between 100 and 10,000 ng/ml, between 1,000 and 10,000 ng/ml, between 5,000 and 10,000 ng/ml, between 0.1 and 5,000 ng/ml, between 1 and 5,000 ng/ml, between 10 and 5,000 ng/ml, between 1,000 and 5,000 ng/ml, or between 2,500 and 5,000 ng/ml.

In some embodiments, the effective amount of the WNT activator for the methods described herein can be between 0.1 and 100 μM. In some embodiments, the effective amount of the WNT activator for the methods described herein can be between 0.1 and 90 μM, between 0.1 and 80 μM, between 0.1 and 70 μM, between 0.1 and 60 μM, between 0.1 and 50 μM, between 0.1 and 40 μM, between 0.1 and 30 μM, between 0.1 and 20 μM, between 0.1 and μM, between 0.1 and 1 μM, and between 0.1 and 0.5 μM. In some embodiments, the effective amount of the WNT activator for the methods described herein can be between 0.5 and 100 μM, between 1 and 100 μM, between 10 and 100 μM, between 20 and 100 μM, between 30 and 100 μM, between 40 and 100 μM, between 50 and 100 μM, between 60 and 100 μM, between 70 and 100 μM, between 80 and 100 μM, and between 90 and 100 μM.

Methods described herein encompass culturing any of the populations of cells in a medium that comprises at least one additional factor, e.g., a MEK inhibitor, a growth factor, leukemia inhibitory factor (LIF), knockout serum replacement (KOSR), fetal bovine serum (FBS), a INK inhibitor, or a combination thereof.

A MEK inhibitor is a chemical or drug that inhibits the mitogen-activated protein kinase kinase enzymes MEK1 and/or MEK2. They can be used to inhibit the MAPK/ERK pathway. MEK is a kinase that phosphorylates mitogen-activated protein kinase (MAPK). As used herein, “MEK” refers to any isoform of MEK (e.g., MEK1 or MEK2). Inhibitors that inhibit either or both of these isoforms are of use. In some embodiments, the MEK inhibitor specifically inhibits MEK and does not substantially inhibit the majority of other mammalian kinases.

Any MEK inhibitor may be used in methods described herein. Exemplary MEK inhibitors include, but are not limited to, GSK1120212, XL518, MEK162, CI-1040, PD0325901, and TAK-733.

It will be understood that the MEK inhibitor should be capable of entering cells in sufficient quantities to inhibit MEK therein, thereby producing formative PSCs. In some embodiments, the MEK inhibitor is added to the cell culture medium at a concentration at least equal to the IC50 of the MEK inhibitor. In some embodiments, the MEK inhibitor is added to the cell culture medium at a concentration between 0.5 and 50 times the IC50 of the MEK inhibitor.

In some embodiments, the MEK inhibitor (e.g., PD0325901) is added to the cell culture medium at a concentration between 0.1 and 100 μM. In some embodiments, the MEK inhibitor (e.g., PD0325901) is added to the cell culture medium at a concentration between 0.1 and μM, between 0.1 and 80 μM, between 0.1 and 70 μM, between 0.1 and 60 μM, between 0.1 and 50 μM, between 0.1 and 40 μM, between 0.1 and 30 μM, between 0.1 and 20 μM, between and 10 μM, between 0.1 and 1 μM, and between 0.1 and 0.5 μM. In some embodiments, the MEK inhibitor (e.g., PD0325901) is added to the cell culture medium at a concentration between and 100 μM, between 1 and 100 μM, between 10 and 100 μM, between 20 and 100 μM, between 30 and 100 μM, between 40 and 100 μM, between 50 and 100 μM, between 60 and 100 μM, between 70 and 100 μM, between 80 and 100 μM, and between 90 and 100 μM.

Leukemia Inhibitory Factor (LIF) belongs to the interleukin-6 cytokine family. LIF binds to a heterodimeric receptor consisting of the low-affinity LIF receptor and gp130, with downstream signals being transmitted through gp130. There are a number of signaling pathways downstream of gp130, including the STAT3, phosphatidylinositol 3-kinase (PI3K) and Ras/Erk pathways.

In some embodiments, LIF protein is added to the medium. In some embodiments, LIF protein is added to the cell culture medium at a concentration between 0.1 and ng/ml (e.g., 10 ng/ml). In some embodiments, the LIF protein is added to the cell culture medium at a concentration between 1 and 10,000 ng/ml, between 10 and 10,000 ng/ml, between 100 and 10,000 ng/ml, between 1,000 and 10,000 ng/ml, between 5,000 and 10,000 ng/ml, between and 5,000 ng/ml, between 1 and 5,000 ng/ml, between 10 and 5,000 ng/ml, between 1,000 and ng/ml, or between 2,500 and 5,000 ng/ml.

Knockout serum replacement (KOSR) is a defined serum-free formulation optimized to grow and maintain undifferentiated embryonic stem cells in culture. It is more stable, more consistent in quality and performs better in the maintenance of undifferentiated status of embryonic stem (ES) cells and induced pluripotent stem (iPS) cells than fetal bovine serum.

In some embodiments, KOSR is added to the medium. In some embodiments, KOSR is added to the cell culture medium at a concentration between 0.1 and 20% (e.g., 10%). In some embodiments, KOSR is added to the cell culture medium at a concentration between 0.1% and 15%, 0.1% and 10%, 0.1% and 5%, 0.1% and 1%, 0.1% and 0.5%, 0.5% and 20%, 1% and 20%, 5% and 20%, 10% and 20%, and 15% and 20%.

Fetal bovine serum (FBS) is the liquid fraction of clotted blood from fetal calves, depleted of cells, fibrin and clotting factors, but containing a large number of nutritional and macromolecular factors essential for cell growth. Bovine serum albumin is the major component of FBS. Growth factors in FBS are essential for the maintenance and growth of cultured cells. FBS also contains a variety of small molecules like amino acids, sugars, lipids, and hormones.

In some embodiments, FBS is added to the medium. In some embodiments, FBS is added to the cell culture medium at a concentration between 0.1 and 20% (e.g., 10%). In some embodiments, FBS is added to the cell culture medium at a concentration between 0.1% and 15%, 0.1% and 10%, 0.1% and 5%, 0.1% and 1%, 0.1% and 0.5%, 0.5% and 20%, 1% and 20%, 5% and 20%, 10% and 20%, and 15% and 20%.

C-JUN N-terminal kinases (JNKs) belong to the mitogen-activated protein kinase (MAPK) family, and that mediate cell responses to various types of extracellular stress insults. They regulate physiological processes such as embryonic development and tissue regeneration, playing roles in cell proliferation and programmed cell death.

In some embodiments, an effective amount of a JNK inhibitor is added to the medium. In some aspects, an effective amount of a JNK inhibitor for culturing the population of cells in methods described herein results in increased growth of the pluripotent cells. Such increased growth may be observed, for example, as a decrease in doubling time of the pluripotent cell population. In some embodiments, a JNK inhibitor is added to the medium at a concentration of from about 0.1 μM to about 20 μM, from about 0.1 μM to about 15 μM, from about 0.1 μM to about 10 μM, from about 0.1 to about 5 μM, from about 0.5 μM to about 20 μM, from about 0.5 μM to about 15 μM, from about 0.5 μM to about 10 μM, or from about 0.5 μM to about 5 μM. In some embodiments, the JNK inhibitor is added to the medium at a concentration of from about 0.1 μM to about 20 μM. In some embodiments, the JNK inhibitor may comprise SP600125.

The population of cells may be cultured in the presence of the at least one additional factor and a FGF activator, a TGF-β activator, and a WNT activator. Alternatively, or in addition to, the population of cells may be cultured in the presence of the at least one additional factor prior to and/or after culturing the population of cells in the presence of a FGF activator, a TGF-β activator, and a WNT activator.

For example, the population of cells may be cultured in the presence of a MEK inhibitor (e.g., PD0325901) prior to culturing the population of cells in the presence of a FGF activator, a TGF-β activator, and a WNT activator. In another example, the population of cells may be cultured in the presence of FBS, a FGF activator, a TGF-β activator, and a WNT activator. In another example, the population of cells may be cultured in the presence of LIF protein, a FGF activator, a TGF-β activator, and a WNT activator. In yet another example, the population of cells may be cultured in the presence of FBS, LIF protein, a FGF activator, a TGF-β activator, and a WNT activator. In yet another example, the population of cells may be cultured in the presence of a INK inhibitor (e.g., SP600125) after culturing the population of cells in the presence of a FGF activator, a TGF-β activator and a WNT activator.

Methods described herein encompass genetic manipulation of any of the populations of cells described herein. A genetic manipulation includes modifying, inserting, or deleting at least one of the genes in the cells.

Genetic manipulation may include transduction with a vector such as a non-integrating vector (e.g., an episomal vector) or an integrating vector (e.g., lentiviral vector). In some embodiments, methods described herein involve genetically manipulating a population of cells using an episomal vector. Accordingly, in some embodiments, the population of cells involved in the methods described herein are gene-modified cells.

A “vector,” as used herein is any nucleic acid vehicle (DNA or RNA) capable of facilitating the transfer of a nucleic acid molecule into cells. In general, vectors include, but are not limited to, episomal vectors, plasmids, phagemids, viral vectors, and other vehicles derived from viral or bacterial sources that have been manipulated by the insertion or incorporation of a target nucleotide sequence. Viral vectors include, but are not limited to vectors comprising nucleotide sequences derived from the genome of the following viruses: retrovirus; lentivirus; adenovirus; adeno-associated virus; SV40-type viruses; polyomaviruses; Epstein-Barr viruses; papilloma viruses; herpes virus; vaccinia virus; polio virus. One can readily employ other vectors not named but known to the art.

Methods described herein encompass reprogramming the population of cells (e.g., the population of somatic cells) to a less differentiated state. Reprogramming, as used herein, refers to a process that alters or reverses the differentiation status of a cell (e.g., a somatic cell), which can be either partially or terminally differentiated. Reprogramming includes complete reversion, as well as partial reversion, of the differentiation status of a cell.

Any method known in the art for reprogramming cells may be used in methods described herein. In general, methods for reprogramming cells comprise culturing the cells in the presence of an agent that alters or reverses the differentiation status of a cell, which may be referred to as a reprogramming agent. In some embodiments, the reprogramming agent is a nucleic acid that alters expression of a gene (e.g., OCT3/4, p53, SOX2, KLF4, L-MYC, and LIN28).

Methods described herein encompass differentiating the population of cells (e.g., formative PSCs) to a more differentiated or specialized state. Formative PSCs of the present invention may be induced to differentiate to obtain desired cell types according to known methods. In general, methods for differentiating cells comprise culturing the cells in the presence of an agent that induces differentiation, which may be referred to as a differentiating agent. In some embodiments, the formative PSCs may be induced to differentiate into primordial germ cells, hematopoietic stem cells, muscle cells, cardiac muscle cells, liver cells, cartilage cells, epithelial cells, urinary tract cells, by culturing such cells in the presence of a differentiating agent under conditions which provide for cell differentiation Accordingly, methods described herein encompass culturing the formative PSCs in the presence of at least one differentiation factor. Any differentiation factor known in the art may be used to differentiate formative PSCs to a more differentiated or specialized state. Examples of differentiation factors include, but are not limited to, growth factors, WNT inhibitors, TGF-β inhibitors, and combinations thereof. In some embodiments, the growth factor is fibroblast growth factor (FGF), Noggin, or a combination thereof. In some embodiments, the WNT inhibitors is IWP2. In some embodiments, the TGF-β inhibitor is SB431542.

### II. Pathway Activators

Aspects of the present disclosure are based on the surprising discovery that stem cells can be captured in discrete formative stages of pluripotency by activation of FGF, TGF-β, and WNT in the stem cells. Accordingly, provided herein are methods for producing formative pluripotent stem cells (PSCs) by culturing a population of cells in the presence of at least one FGF activator, at least one TGF-β activator, and at least one WNT activator.

### (a) FGF Activators

FGFs are a family of cell signaling proteins that play a role in a wide range of cellular processes. FGF family members comprise FGF proteins (e.g., FGF1, FGF2, FGB, FGF4, FGFS, FGF6, FGF7, FGF8, FGF9, FGF10, FGF11, FGF12, FGF13, FGF14, FGF15, FGF16, FGF17, FGF18, FGF19, FGF20, FGF21, FGF22, and FGF23) and FGF receptor (FGFR) proteins (e.g., FGFR1, FGFR2, FGFR3, FGFR4, and FGFRL1). Sequences of genes and proteins in the FGF pathway are known in the art, and may be obtained from publicly available databases.

Activators for any of FGF family members can be used in the culturing methods described herein. In some embodiments, FGF activators used herein are specific to one FGF family member, e.g., specific to FGF or FGFR. In some embodiments, FGF activators are universal to two or more FGF family members, e.g., universal to FGF and FGFR. In some embodiments, FGF activators used herein are specific to FGF2.

As used herein, the term “FGF activator” refers to a molecule that partially or fully enhances, increases, or stimulates a biological activity of a FGF protein. Suitable FGF activators include, but are not limited to, proteins, nucleic acids, small molecules, or combinations thereof. Methods for identifying activators of FGF may comprise contacting FGF with a candidate FGF activator and measuring a detectable change in one or more biological activities typically associated with FGF.

As used herein, the term “FGF” refers to a FGF polypeptide having the same or similar bioactivity of a wild-type FGF polypeptide. A FGF polypeptide may have an amino acid sequence that is at least 70% or more (e.g., at least 75%, at least 80%, at least 85%, at least 90%, at least 95%, at least 97%, at least 99%, or 100%) identical to that of a wild-type FGF polypeptide, and is capable of trigger FGF signaling pathway.

Wild-type FGF sequences (e.g., sequences of FGF2) of various species are available on the world wide web from the NCBI, including human, mouse, and rat. For example, the nucleotide sequence encoding an isoform of human FGF2 is available at NCBI under Accession No. NM_002006.4 (SEQ ID NO: 1) and its corresponding amino acid sequence is under Accession No. NP_001997.5 (SEQ ID NO: 2).

A FGF activator can be a molecule of any type that enhances the signaling associated with at least one FGF family members (e.g., FGFs or FGFRs) in a cell, for example, either by increasing transcription or translation of a FGF family member, or by increasing FGF activity, or both. In some embodiments, the FGF activator may act directly by interacting with a FGF receptor or indirectly by interacting with one or more intracellular components of the FGF signaling pathway.

In some embodiments, FGF activators as described herein may increase FGF signaling in cells (e.g., ESCs or iPSCs) by at least 20% or more, e.g., 30%, 40%, 50%, 60%, 70%, 80%, 90%, 95% or above. The increased FGF signaling in the presence of at least one FGF activator can be determined by conventional methods, e.g., using protein assays such as ELISA or Western blot.

In some embodiments, the FGF activator increases activity of the FGF pathway to levels sufficient to produce formative PSCs. In some embodiments, FGF activators can be used to characterize or explore the stages and/or mechanism related to the pluripotency continuum.

It will be understood that the FGF activator should be capable of entering cells in sufficient quantities to increase FGF activity, thereby producing formative PSCs. In some embodiments, the FGF activator is added to the cell culture medium at a concentration between and 10,000 ng/ml. In some embodiments, the FGF activator is added to the cell culture medium at a concentration between 1 and 10,000 ng/ml, between 10 and 10,000 ng/ml, between 100 and ng/ml, between 1,000 and 10,000 ng/ml, between 5,000 and 10,000 ng/ml, between 0.1 and ng/ml, between 1 and 5,000 ng/ml, between 10 and 5,000 ng/ml, between 1,000 and 5,000 ng/ml, or between 2,500 and 5,000 ng/ml.

In some embodiments, FGF activators used for the methods described herein are cell-permeable. In some embodiments, FGF activators may be added to a cell culture medium prior to using the cell culture medium to culture the cells. In some embodiments, FGF activators may be added to a cell culture medium during culturing of the cells.

In some embodiments, the FGF activator is a protein or a small molecule. In some embodiments, the FGF activator is a biologically active FGF protein, e.g., FGF2. In some examples, the FGF protein (e.g., FGF2 protein) may be added to the cell culture medium at a concentration between 0.1 and 10,000 ng/ml. In some embodiments, the FGF protein is added to the cell culture medium at a concentration between 0.1 and 10,000 ng/ml. In some embodiments, the FGF protein is added to the cell culture medium at a concentration between 1 and 10,000 ng/ml, between 10 and 10,000 ng/ml, between 100 and 10,000 ng/ml, between 1,000 and 10,000 ng/ml, between 5,000 and 10,000 ng/ml, between 0.1 and 5,000 ng/ml, between 1 and 5,000 ng/ml, between 10 and 5,000 ng/ml, between 1,000 and 5,000 ng/ml, or between 2,500 and 5,000 ng/ml.

Any of the proteins to be used in methods described herein may be isolated from naturally occurring sources (e.g., mammalian cells that naturally produce the protein), produced in eukaryotic or prokaryotic cells using recombinant expression technology, or chemically synthesized. Soluble, biologically active FGF proteins may be prepared in purified form using methods known in the art.

In some embodiments, the FGF activator is a small molecule, such as a small organic molecule, which typically has a molecular weight less than 5,000 kDa. Suitable small molecules include those that bind to one or more family members of FGF (e.g., FGF and/or FGFR) or a fragment thereof, and that are known in the art or identified by methods such as screening large libraries of compounds.

### (b) TGF-β Activators

TGF-β is a multifunctional cytokine belonging to the transforming growth factor superfamily that comprises TGF-βs (e.g., TGF-β1, TGF-β2, and TGF-β3), Activins (e.g., Activin-A, Activin-B, and Activin-C), bone morphogenetic proteins (BMPs), and TGF-β receptors (TGFRs) (e.g., type I, type II, and type III receptors). Sequences of genes and proteins in the TGF-β pathway are known in the art, and may be obtained from publically available databases.

Activators for any of TGF-β family members can be used in the culturing methods described herein. In some embodiments, TGF-β activators used herein are specific to one TGF-β family member, e.g., specific to a TGF-β or an Activin (e.g., TGF-β2 or Activin-A). In some embodiments, TGF-β activators are universal to two or more TGF-β family members, e.g., universal to a TGF-β and an Activin (e.g., TGF-β2 or Activin-A).

As used herein, the term “TGF-β activator” refers to a molecule that partially or fully enhances, increases, or stimulates a biological activity of a TGF-β protein. Suitable TGF-β activators include, but are not limited to, proteins, nucleic acids, small molecules, or combinations thereof. Methods for identifying activators of TGF-β may comprise contacting TGF-β with a candidate TGF-β activator and measuring a detectable change in one or more biological activities typically associated with TGF-β.

As used herein, the term “TGF-β” refers to a TGF-β polypeptide having the same or similar bioactivity of a wild-type TGF-β polypeptide. A TGF-β polypeptide may have an amino acid sequence that is at least 70% or more (e.g., at least 75%, at least 80%, at least 85%, at least 90%, at least 95%, at least 97%, at least 99%, or 100%) identical to that of a wild-type TGF-β polypeptide, and is capable of trigger TGF-β signaling pathway.

Wild-type TGF-β sequences (e.g., sequences of TGF-β1, TGF-β2, or TGF-β3) of various species are available on the world wide web from the NCBI, including human, mouse, and rat. For example, the nucleotide sequence encoding an isoform of human TGF-β2 is available at NCBI under Accession No. NM_001024847.2 (SEQ ID NO: 3) and its corresponding amino acid sequence is under Accession No. NP_001020018.1 (SEQ ID NO: 4).

As used herein, the term “Activin” refers to an Activin polypeptide having the same or similar bioactivity of a wild-type Activin polypeptide. An Activin polypeptide may have an amino acid sequence that is at least 70% or more (e.g., at least 75%, at least 80%, at least 85%, at least 90%, at least 95%, at least 97%, at least 99%, or 100%) identical to that of a wild-type Activin polypeptide, and is capable of trigger TGF-β signaling pathway.

Wild-type Activin sequences (e.g., sequences of Activin-A, Activin-B, and Activin-C) of various species are available on the world wide web from the NCBI, including human, mouse, and rat. For example, the nucleotide sequence encoding an isoform of human Activin-A is available at NCBI under Accession No. NM_002192.3 (SEQ ID NO: 5) and its corresponding amino acid sequence is under Accession No. NP_002183.1 (SEQ ID NO: 6).

A TGF-β activator can be a molecule of any type that enhances the signaling associated with at least one TGF-β family members (e.g., TGF-β, Activin, BMP, or FGFR) in a cell, for example, either by increasing transcription or translation of a TGF-β family member, or by increasing TGF-β activity, or both. In some embodiments, the TGF-β activator may act directly by interacting with a TGF-β receptor or indirectly by interacting with one or more intracellular components of the TGF-β signaling pathway.

In some embodiments, TGF-β activators as described herein may increase TGF-β signaling in cells (e.g., ESCs or iPSCs) by at least 20% or more, e.g., 30%, 40%, 50%, 60%, 70%, 80%, 90%, 95% or above. The increased TGF-β signaling in the presence of at least one TGF-β activator can be determined by conventional methods, e.g., using protein assays such as ELISA or Western blot.

In some embodiments, the TGF-β activator increases activity of the TGF-β pathway to levels sufficient to produce formative PSCs. In some embodiments, TGF-β activators can be used to characterize or explore the stages and/or mechanism related to the pluripotency continuum.

It will be understood that the TGF-β activator should be capable of entering cells in sufficient quantities to increase TGF-β activity, thereby producing formative PSCs. In some embodiments, the TGF-β activator is added to the cell culture medium at a concentration between 0.1 and 10,000 ng/ml. In some embodiments, the TGF-β activator is added to the cell culture medium at a concentration between 1 and 10,000 ng/ml, between 10 and 10,000 ng/ml, between 100 and 10,000 ng/ml, between 1,000 and 10,000 ng/ml, between 5,000 and 10,000 ng/ml, between 0.1 and 5,000 ng/ml, between 1 and 5,000 ng/ml, between 10 and 5,000 ng/ml, between 1,000 and 5,000 ng/ml, or between 2,500 and 5,000 ng/ml.

In some embodiments, TGF-β activators used for the methods described herein are cell-permeable. In some embodiments, TGF-β activators may be added to a cell culture medium prior to using the cell culture medium to culture the cells. In some embodiments, TGF-β activators may be added to a cell culture medium during culturing of the cells.

In some embodiments, the TGF-β activator is a protein or a small molecule. In some embodiments, the TGF-β activator is a biologically active TGF-β protein, e.g., FGF2 protein, Activin-A protein, TGF-β1 protein, or a combination thereof. In some examples, the TGF-β protein (e.g., FGF2 protein, Activin-A protein, TGF-β1 protein, or a combination thereof) may be added to the cell culture medium at a concentration between 0.1 and 10,000 ng/ml. In some embodiments, the TGF-β activator is added to the cell culture medium at a concentration between 1 and 10,000 ng/ml, between 10 and 10,000 ng/ml, between 100 and 10,000 ng/ml, between 1,000 and 10,000 ng/ml, between 5,000 and 10,000 ng/ml, between 0.1 and 5,000 ng/ml, between 1 and ng/ml, between 10 and 5,000 ng/ml, between 1,000 and 5,000 ng/ml, or between 2,500 and ng/ml.

Any of the proteins to be used in methods described herein may be isolated from naturally occurring sources (e.g., mammalian cells that naturally produce the protein), produced in eukaryotic or prokaryotic cells using recombinant expression technology, or chemically synthesized. Soluble, biologically active TGF-β proteins may be prepared in purified form using methods known in the art.

In some embodiments, the TGF-β activator is a small molecule, such as a small organic molecule, which typically has a molecular weight less than 5,000 kDa. Suitable small molecules include those that bind to one or more family members of TGF-β or a fragment thereof, and that are known in the art or identified by methods such as screening large libraries of compounds.

### (c) WNT Activators

WNTs are a family of secreted proteins important for a wide array of developmental and physiological processes. WNT family members comprise WNT1, WNT2, WNT2b (also called WNT13), WNT3, WNT3a, WNT 4, WNT 5a, WNT 5b, WNT6, WNT7a, WNT7b, WNT8a, WNT 8b, WNT9a, WNT9b, WNT10a, WNT10b, WNT11, and WNT16. Sequences of genes and proteins in the WNT pathway are known in the art and may be obtained from publicly available databases.

Activators for any of WNT family members can be used in the culturing methods described herein. In some embodiments, WNT activators used herein are specific to one WNT family member, e.g., specific to WNT1, WNT2, WNT3, or WNT3a. In some embodiments, WNT activators are universal to two or more WNT family members, e.g., universal to WNT3 and WNT3a. In some embodiments, WNT activators used herein are specific to WNT3a.

As used herein, the term “WNT activator” refers to a molecule that partially or fully enhances, increases, or stimulates a biological activity of a WNT protein. Suitable WNT activators include, but are not limited to, proteins, nucleic acids, small molecules, or combinations thereof. Methods for identifying activators of WNT may comprise contacting WNT with a candidate WNT activator and measuring a detectable change in one or more biological activities typically associated with WNT.

As used herein, the term “WNT” refers to a WNT polypeptide having the same or similar bioactivity of a wild-type WNT polypeptide. A WNT polypeptide may have an amino acid sequence that is at least 70% or more (e.g., at least 75%, at least 80%, at least 85%, at least 90%, at least 95%, at least 97%, at least 99%, or 100%) identical to that of a wild-type WNT polypeptide, and is capable of trigger WNT signaling pathway.

Wild-type WNT sequences (e.g., sequences of WNT1, WNT2, WNT3, or WNT3a) of various species are available on the world wide web from the NCBI, including human, mouse, and rat. For example, the nucleotide sequence encoding an isoform of human WNT3a is available at NCBI under Accession No. NM_033131.3 (SEQ ID NO: 7) and its corresponding amino acid sequence is under Accession No. NP_149122.1 (SEQ ID NO: 8).

A WNT activator can be a molecule of any type that enhances the signaling associated with at least one WNT family members (e.g., WNT1, WNT2, WNT3, or WNT3a) in a cell, for example, either by increasing transcription or translation of a WNT family member, or by increasing WNT activity, or both. In some embodiments, the WNT activator may act directly by interacting with a WNT receptor or indirectly by interacting with one or more intracellular components of the WNT signaling pathway such as β-catenin, a kinase or phosphatase that acts on β-catenin, a transcription factor that assembles with β-catenin.

In some embodiments, WNT activators as described herein may increase WNT signaling in cells (e.g., ESCs or iPSCs) by at least 20% or more, e.g., 30%, 40%, 50%, 60%, 70%, 80%, 90%, 95% or above. The increased WNT signaling in the presence of at least one WNT activator can be determined by conventional methods, e.g., using protein assays such as ELISA or Western blot.

In some embodiments, the WNT activator increases activity of the WNT pathway to levels sufficient to produce formative PSCs. In some embodiments, WNT activators can be used to characterize or explore the stages and/or mechanism related to the pluripotency continuum.

It will be understood that the WNT activator should be capable of entering cells in sufficient quantities to increase WNT activity, thereby producing formative PSCs. In some embodiments, the WNT activator is added to the cell culture medium at a concentration between and 10,000 ng/ml. In some embodiments, the WNT activator is added to the cell culture medium at a concentration between 1 and 10,000 ng/ml, between 10 and 10,000 ng/ml, between 100 and 10,000 ng/ml, between 1,000 and 10,000 ng/ml, between 5,000 and 10,000 ng/ml, between and 5,000 ng/ml, between 1 and 5,000 ng/ml, between 10 and 5,000 ng/ml, between 1,000 and ng/ml, or between 2,500 and 5,000 ng/ml.

In some embodiments, WNT activators used for the methods described herein are cell permeable. In some embodiments, WNT activators may be added to a cell culture medium prior to using the cell culture medium to culture the cells. In some embodiments, WNT activators may be added to a cell culture medium during culturing of the cells.

In some embodiments, the WNT activator is a protein or a small molecule. In some embodiments, the WNT activator is a biologically active WNT protein, e.g., WNT3 a protein. In some examples, the WNT protein (e.g., WNT3a protein) may be added to the cell culture medium at a concentration between 0.1 and 10,000 ng/ml. In some embodiments, the WNT protein is added to the cell culture medium at a concentration between 0.1 and 10,000 ng/ml. In some embodiments, the WNT protein is added to the cell culture medium at a concentration between 1 and 10,000 ng/ml, between 10 and 10,000 ng/ml, between 100 and 10,000 ng/ml, between 1,000 and 10,000 ng/ml, between 5,000 and 10,000 ng/ml, between 0.1 and 5,000 ng/ml, between 1 and ng/ml, between 10 and 5,000 ng/ml, between 1,000 and 5,000 ng/ml, or between 2,500 and ng/ml.

Any of the proteins to be used in methods described herein may be isolated from naturally occurring sources (e.g., mammalian cells that naturally produce the protein), produced in eukaryotic or prokaryotic cells using recombinant expression technology, or chemically synthesized. Soluble, biologically active WNT proteins may be prepared in purified form using methods known in the art.

In some embodiments, the WNT activator is a small molecule, such as a small organic molecule, which typically has a molecular weight less than 5,000 kDa. Suitable small molecules include those that bind to one or more family members of WNT (e.g., WNT1, WNT 2, WNT3, and/or WNT3a) or a fragment thereof, and that are known in the art or identified by methods such as screening large libraries of compounds.

In some embodiments, the WNT activator is a glycogen synthase kinase 3 (GSK3) inhibitor. GSK3 is a serine/threonine kinase that has been identified as a regulator of glucose metabolism. As used herein, “GSK3” refers to either or both isoforms of GSK3 (GSK3a and GSK3(3). Inhibitors that inhibit either or both of these isoforms are of use. In some embodiments, the GSK3 inhibitor specifically inhibits GSK3 and does not substantially inhibit the majority of other mammalian kinases.

Any GSK3 inhibitor may be used in methods described herein. Exemplary GSK3 inhibitors include, but are not limited to, BIO, AR-A014418, SB 216763, SB-415286, CHIR98014 (CT98014), CHIR98023 (CT98023), CHIR99021 (CT99021), and CHIR99021 trihydrochloride.

It will be understood that the GSK3 inhibitor should be capable of entering cells in sufficient quantities to inhibit GSK3 therein, thereby producing formative PSCs. In some embodiments, the GSK3 inhibitor is added to the cell culture medium at a concentration at least equal to the IC50 of the GSK3 inhibitor. In some embodiments, the GSK3 inhibitor is added to the cell culture medium at a concentration between 0.5 and 50 times the IC50 of the GSK3 inhibitor.

In some embodiments, the GSK3 inhibitor (e.g., CHIR99021) is added to the cell culture medium at a concentration between 0.1 and 100 μM. In some embodiments, the GSK3 inhibitor (e.g., CHIR99021) is added to the cell culture medium at a concentration between 0.1 and 90 μM, between 0.1 and 80 μM, between 0.1 and 70 μM, between 0.1 and 60 μM, between and 50 μM, between 0.1 and 40 μM, between 0.1 and 30 μM, between 0.1 and 20 μM, between and 10 μM, between 0.1 and 1 μM, and between 0.1 and 0.5 μM. In some embodiments, the GSK3 inhibitor (e.g., CHIR99021) is added to the cell culture medium at a concentration between and 100 μM, between 1 and 100 μM, between 10 and 100 μM, between 20 and 100 μM, between 30 and 100 μM, between 40 and 100 μM, between 50 and 100 μM, between 60 and 100 μM, between 70 and 100 μM, between 80 and 100 μM, and between 90 and 100 μM.

### III. In Vitro Culture Systems Comprising Formative Pluripotent Stem Cells (PSCs)

Aspects of the present disclosure provide an in vitro culture system comprising formative pluripotent stem cells (PSCs), which can be prepared by any of the methods also described herein.

Any proportion of cells may be in the formative stage of pluripotency in the in vitro culture systems described herein. In some embodiments, the in vitro culture system comprises at least 10% or more of formative PSCs, including, e.g., at least about 20%, at least about 30%, at least about 40%, at least about 50%, at least about 60%, at least about 70%, at least about 80%, at least about 90% or more of formative PSCs.

In some embodiments, in vitro culture systems provided herein may comprise one or more activators described herein, e.g., a fibroblast growth factor (FGF) activator, a transforming growth factor beta (TGF-β) activator, and a WNT activator.

In some embodiments, in vitro culture systems provided herein may comprise one or more additional components such as feeder cells and/or scaffolds. Any feeder cells and/or scaffolds known in the art may be used in methods and/or in vitro culture systems described herein.

### IV. Applications

Any of the methods and/or in vitro culture systems involving formative pluripotent stem cells (PSCs) as described herein may be used to produce cells of any type. For example, formative PSCs produced as described herein may be induced to differentiate into primordial germ cells, hematopoietic stem cells, muscle cells, cardiac muscle cells, liver cells, cartilage cells, epithelial cells, and urinary tract cells.

Any of the methods and/or in vitro culture systems involving formative PSCs as described herein may be used for non-clinical purposes, e.g., for research purposes. In some embodiments, methods and/or in vitro culture systems described herein may be used to study the behavior of stem cells (e.g., the discovery of novel biological pathways or processes involved in pluripotency of stem cells).

In some embodiments, methods and/or in vitro culture systems involving formative PSCs as described herein may be used to determine whether a candidate molecule (e.g., a compound) is capable of altering the pluripotency stage of the formative PSCs in the in vitro culture system. For example, the candidate molecule can be added into the in vitro culture system as described herein. After being cultured under suitable conditions for a suitable period, the stage of pluripotency of the cells in the culture system can be compared with a control culture system that does not contain the candidate molecule. If the stage of pluripotency in the presence of the candidate molecule is altered as compared to that in the absence of the candidate molecule, it indicates that the candidate molecule may alter pluripotency.

### V. Kits for Producing Formative Pluripotent Stem Cells (PSCs)

The present disclosure also provides kits for use in producing the formative PSCs described herein such as embryonic stem cells (ESCs) or induced pluripotent stem cells (iPSCs). Such kits can include one or more containers comprising one or more activators described herein, e.g., a fibroblast growth factor (FGF) activator, a transforming growth factor beta (TGF-β) activator, and a WNT activator, and optionally, a population of cells (e.g., a population of reproductive cells or a population of somatic cells). Kits described herein may further comprise a reprogramming agent suitable for producing iPSCs. Kits described herein may further comprise a cell culture medium suitable for culturing a population of cells.

In some embodiments, the kit can comprise instructions for use in accordance with any of the methods described herein. The included instructions can comprise a description of culturing a population of cells in a medium comprising a FGF pathway activator, a TGF-β pathway activator, and a WNT pathway activator as described herein. The kit may further comprise a description of obtaining a population of cells, e.g., obtaining a population of cells comprising blastocysts produced by natural mating, in vitro fertilization (IVF), intracytoplasmic sperm injection (ICSI) and somatic cell nuclear transfer (SCNT). In still other embodiments, the instructions may comprise a description of culturing the formative PSCs in the presence of at least one differentiation factor, thereby producing differentiated cells such as primordial germ cells, cardiomyocytes or neurons.

The instructions relating to the use of a pathway activator described herein generally include information as to dosage, and dosing schedule for the intended production of formative PSCs (e.g., ESCs or iPSCs). The containers may be unit doses, bulk packages (e.g., multi-dose packages) or subunit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. A kit may have a sterile access port (e.g., the container may be a vial having a stopper pierceable by a hypodermic injection needle).

Kits may optionally provide additional components such as buffers and interpretive information. Normally, the kit comprises a container and a label or package insert(s) on or associated with the container. In some embodiments, the invention provides articles of manufacture comprising contents of the kits described above.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### VI. General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### EXAMPLES

In order that the invention described may be more fully understood, the following examples are set forth. The examples described in this application are offered to illustrate the methods and compositions provided herein and are not to be construed in any way as limiting their scope.

### Example 1. De Novo Derivation of Intermediate PSCs from Mouse Blastocysts

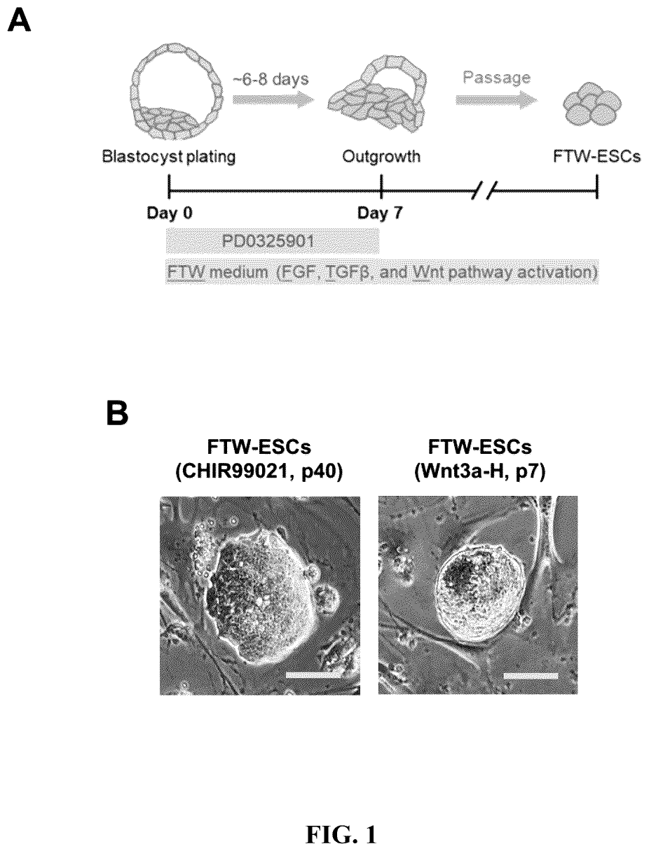

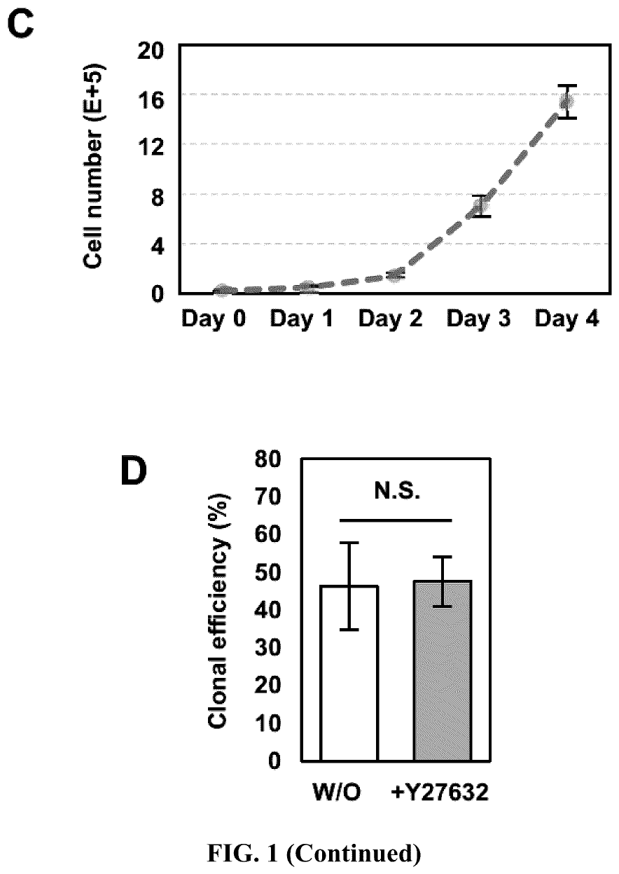

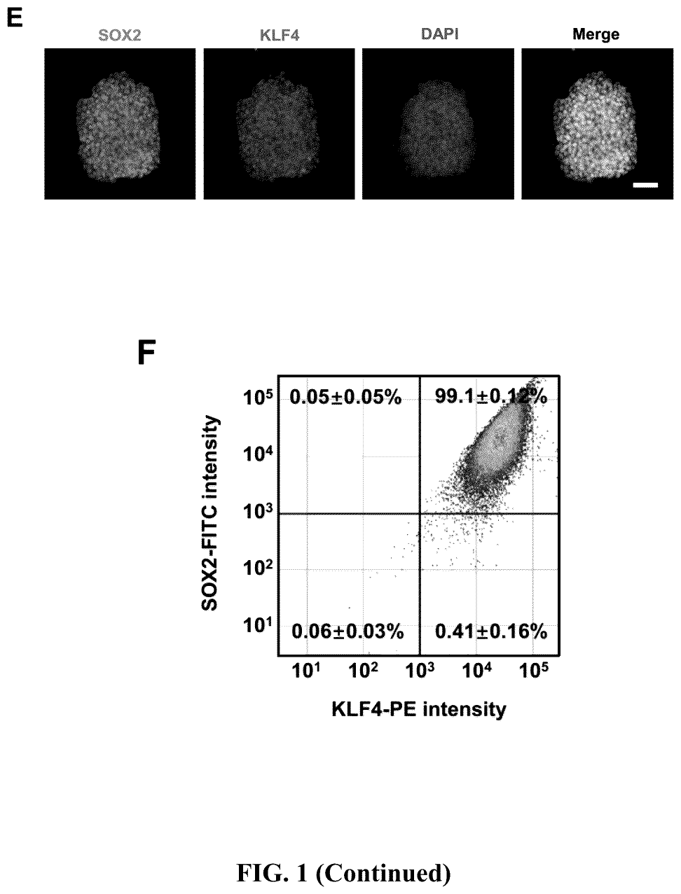

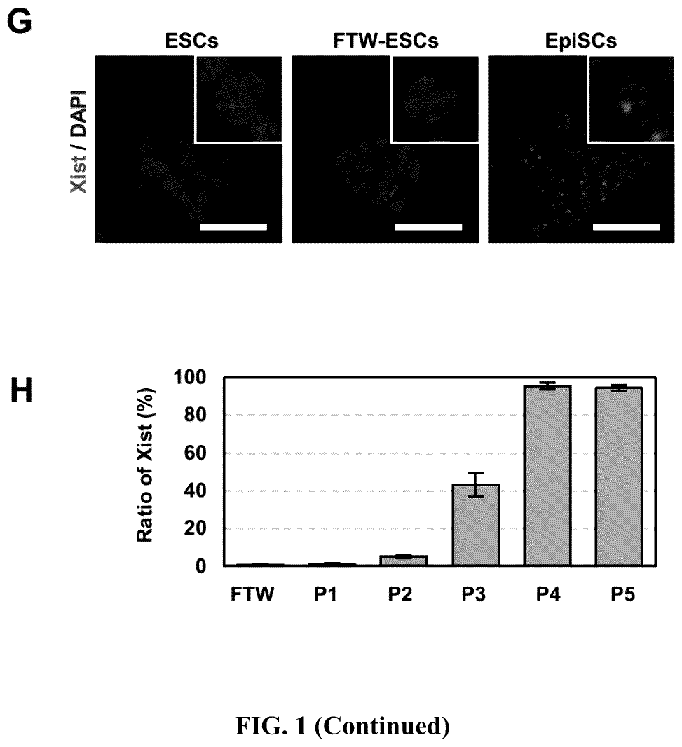

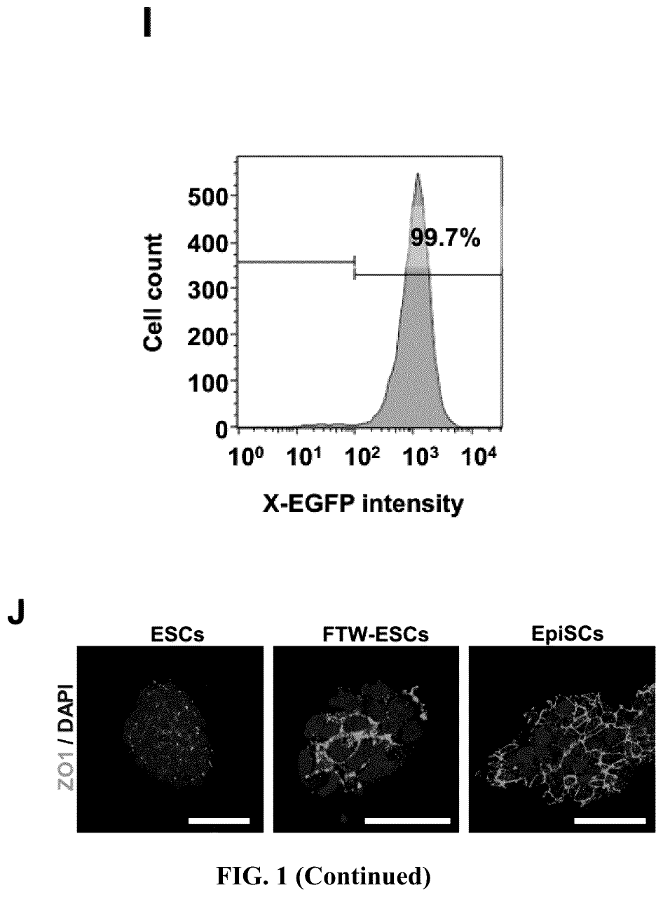

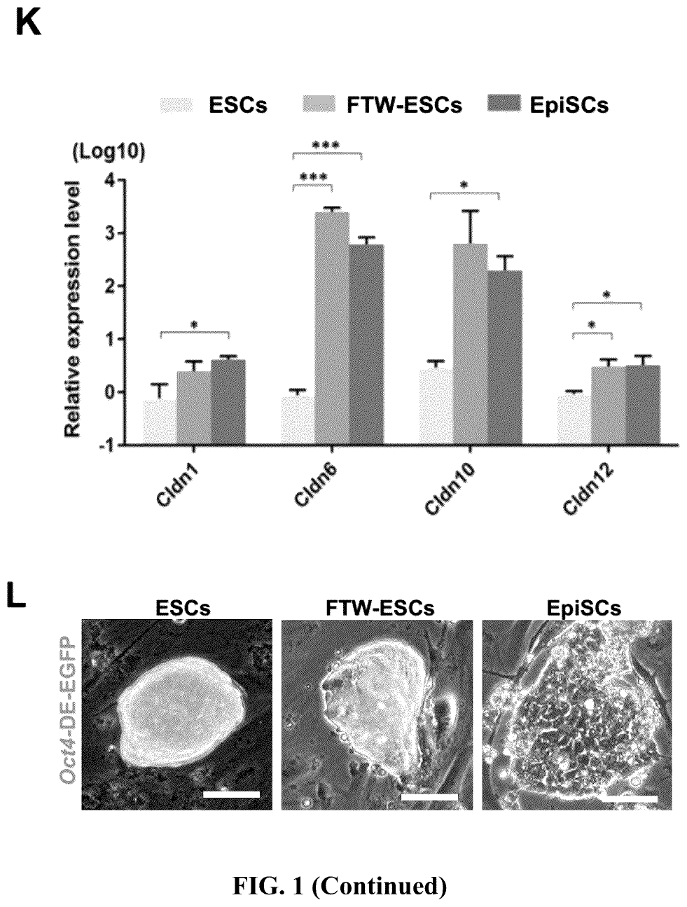

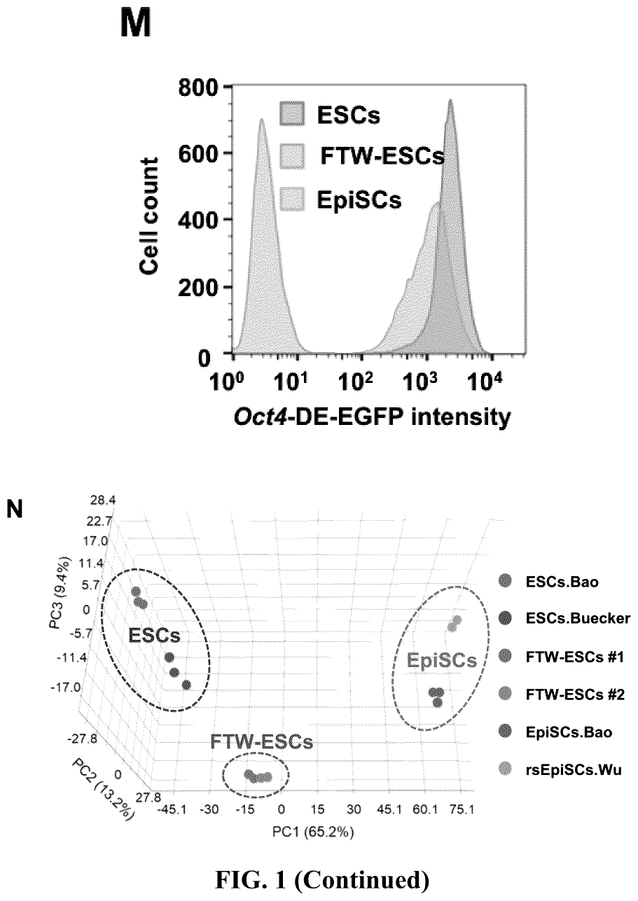

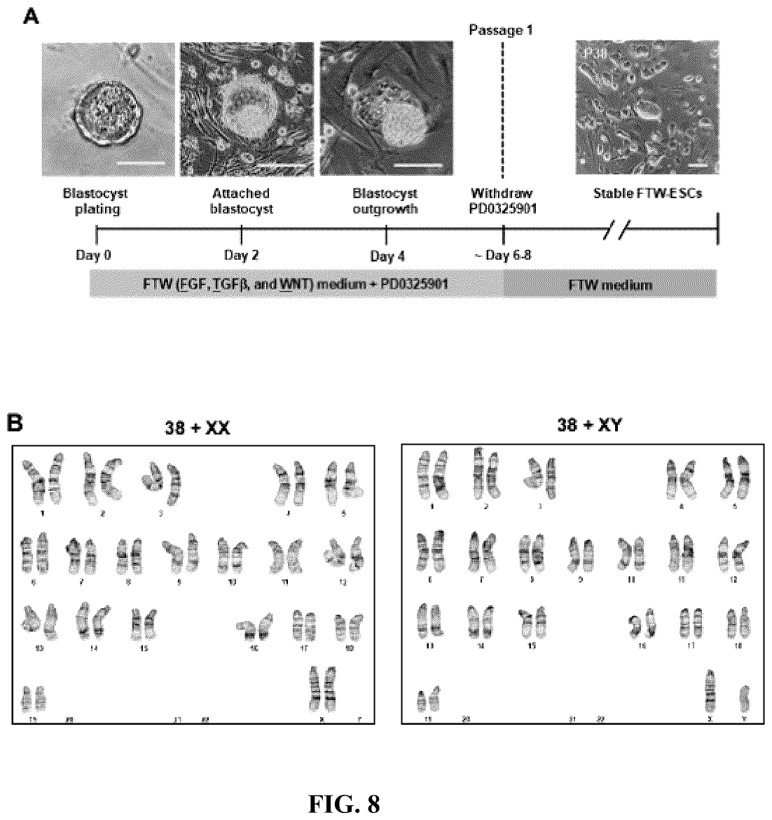

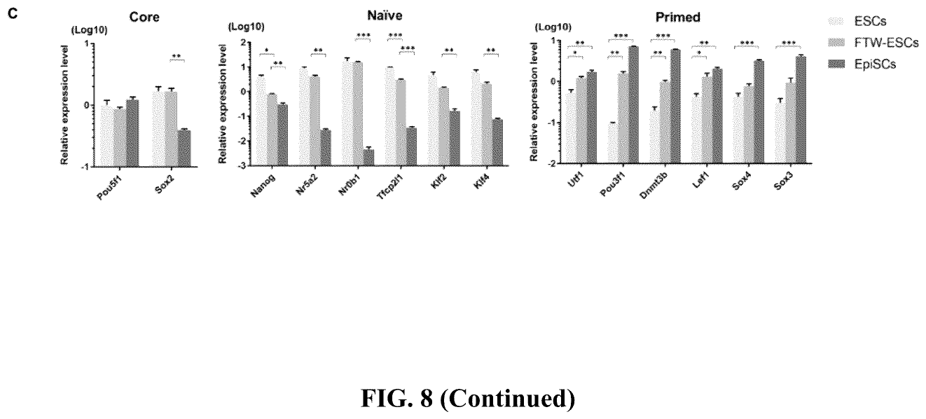

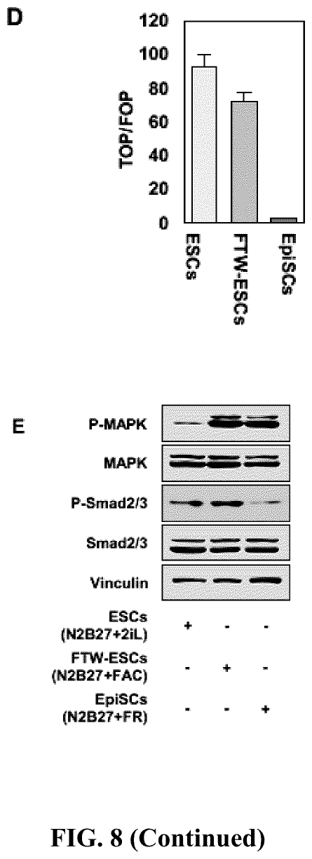

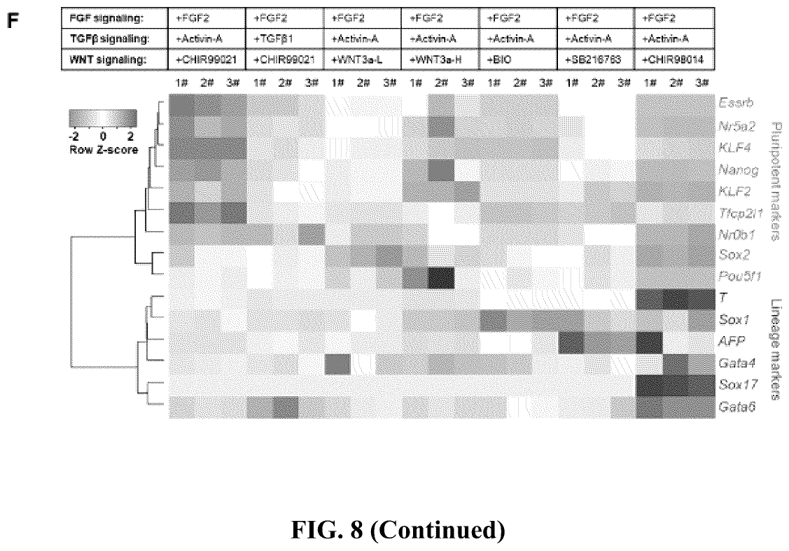

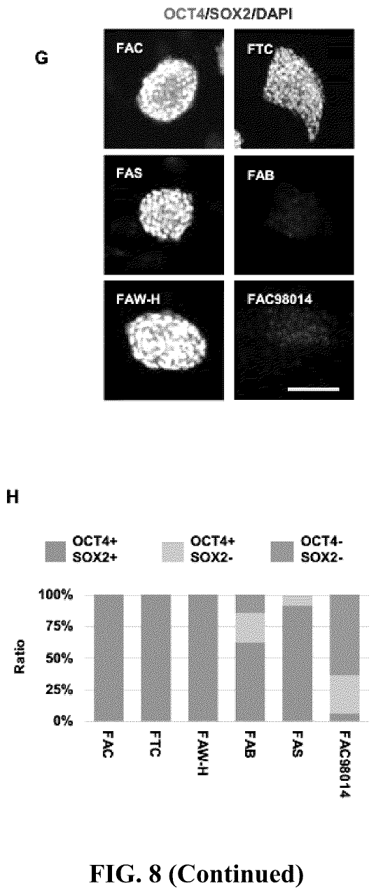

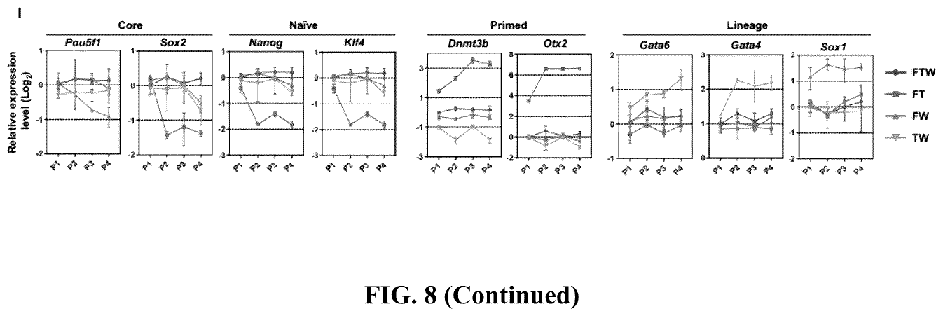

A culture condition containing FGF2, Activin-A, and a GSK3 inhibitor (CHIR99021) was previously used to generate intermediate PSCs (FAC-PSCs) via conversion from naïve ESCs and by somatic cell reprograming (Tsukiyama and Ohinata, PLoS ONE, 9, e953292014). Whether FAC-PSCs could be directly derived from mouse embryos remained unknown. Therefore, de novo derivation from post-implantation epiblasts was tested. Epiblasts from E5.25, E5.5 and E6.25 embryos were manually isolated and cultured in FAC medium on mitotically inactivated mouse embryonic fibroblasts (MEFs). Most epiblasts attached to MEFs but either differentiated or died before passaging (Table 1). Whether PSCs could be derived from preimplantation embryos by plating whole E3.5 blastocysts on MEFs in FAC medium was then tested. After the first passage, although some PSC-like colonies appeared, most colonies were of trophectoderm (TE) or primitive endoderm (PE) origin, which is likely due to FGF2 supplementation. To inhibit the proliferation of TE and PE cells, we added a MEK inhibitor (PD0325901) to FAC culture during the first 6-8 days and removed it following the first passage (FIG. 1A). This resulted in a pure population of PSC-like colonies and stable cell lines (FIG. 1B and FIG. 8A). Supplementation with a high concentration of recombinant WNT3a protein (500 ng/ml) could supplant CHIR99021 in derivation (FIG. 1B), but a lower concentration of WNT3a (50 ng/ml) led to gradual differentiation of PSC-like colonies (data not shown). Using this method, we could derive PSCs from several inbred and outbred genetic backgrounds with high efficiency (Table 1).

TABLE 1
FTW-ESC derivation efficiency

| | | Embryo | derived | |
|---|---|---|---|---|
| Strain | Developmental | numbers | cell lines | Efficiency |
| | stage | # | # | % |
| C57BL/6 | Blastocyst | 36 | 30 | 83.33% |
| B6(Cg)-Tyrc-2J/J | Blastocyst | 11 | 9 | 81.81% |
| 129S1/SvlmJ | Blastocyst | 16 | 11 | 68.75% |
| CD1 (ICR) | Blastocyst | 9 | 5 * | 55.56% |
| C57BL/6 | E5.25 | 8 | 0 | |
| C57BL/6 | E5.5 | 8 | 0 | |
| CD1 (ICR) | E5.5 | 8 | 0 | |
| CD1 (ICR) | E6.25 | 24 | 0 | |

* Spontaneous differentiation observed during initial passages.

Blastocyst-derived FAC-PSCs exhibited colony morphology intermediate of dome-shaped ESCs and flattened EpiSCs (FIG. 1B and FIG. 8A). They maintained stable growth kinetics and normal karyotype after extended culture (FIG. 1C and FIG. 8B) and showed high clonal efficiency with or without ROCK inhibitor (Y27632) treatment (FIG. 1D). Additionally, they expressed pluripotency related genes at both the transcript and protein levels, and displayed homogeneity (FIGS. 1E-1F and FIG. 8C).

Activation of WNT/β-Catenin, FGF/Erk and TGF-β/Smad pathways in de novo derived FAC-PSCs were confirmed by a luciferase-based TopFlash reporter assay, and western blots for phosphor-ERK and phosphor-SMAD2 levels, respectively (FIGS. 8D-8E). Another GSK3 inhibitor (SB216763) and high concentration (500 ng/ml) of WNT3a could substitute CHIR99021 for maintaining the undifferentiated status, while two other GSK3 inhibitors (BIO and CHIR98014) and a lower concentration of WNT3a (50 ng/ml) resulted in differentiation (FIGS. 8F-8H). Additionally, Activin-A and TGF-β1 could be used interchangeably (FIG. 8F and FIG. 8H). Subtraction of FGF2, Activin-A/TGF-β1, or WNT3a/CHIR99021/SB216763 from culture either resulted in downregulation of core and/or naïve pluripotency markers, or upregulation of lineage and/or primed markers (FIG. 8I). Therefore, activation of FGF and TGF-β/Smad pathways, and high WNT/β-Catenin signaling are needed to stabilize the blastocyst-derived PSCs, herein referred to as FTW-ESCs.

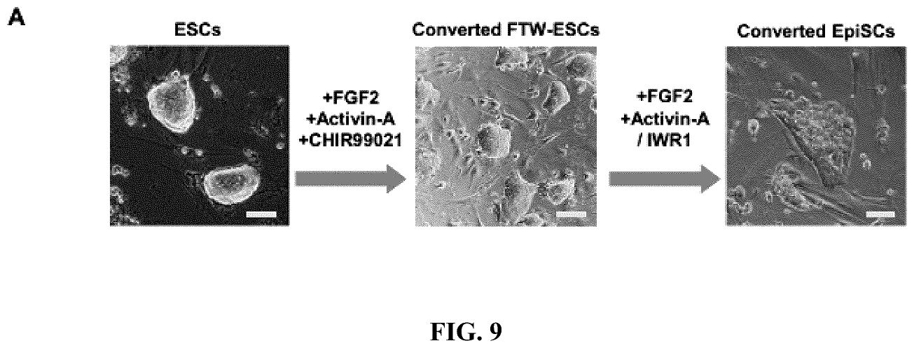

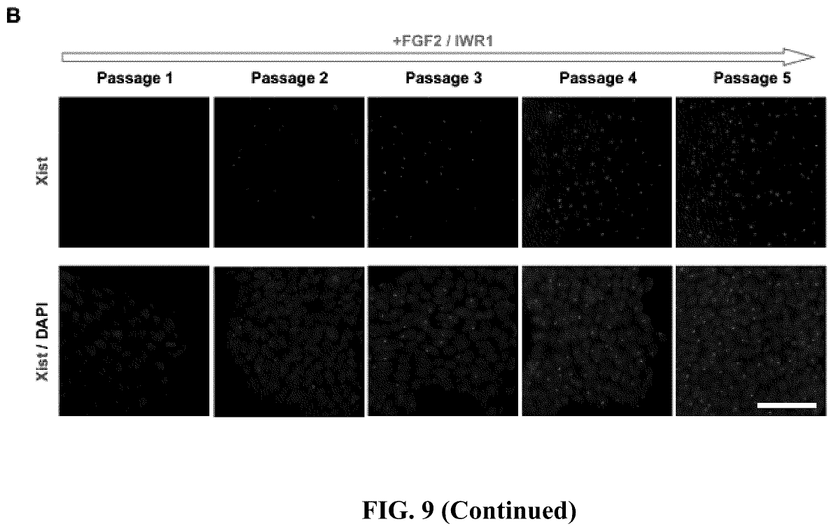

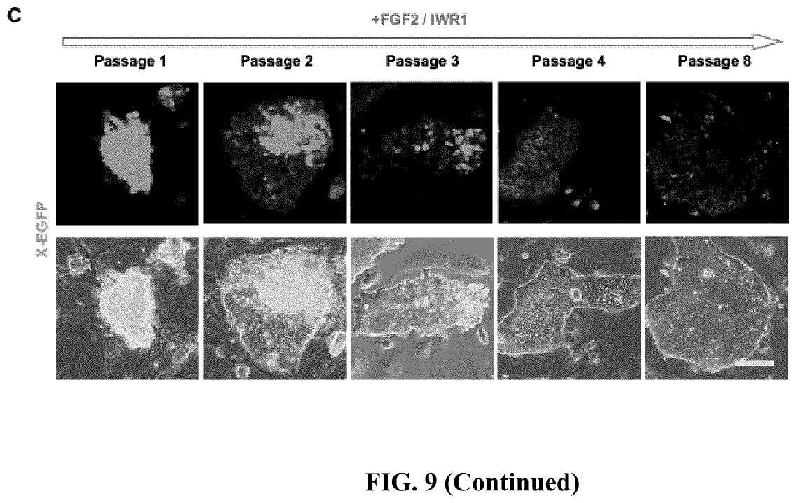

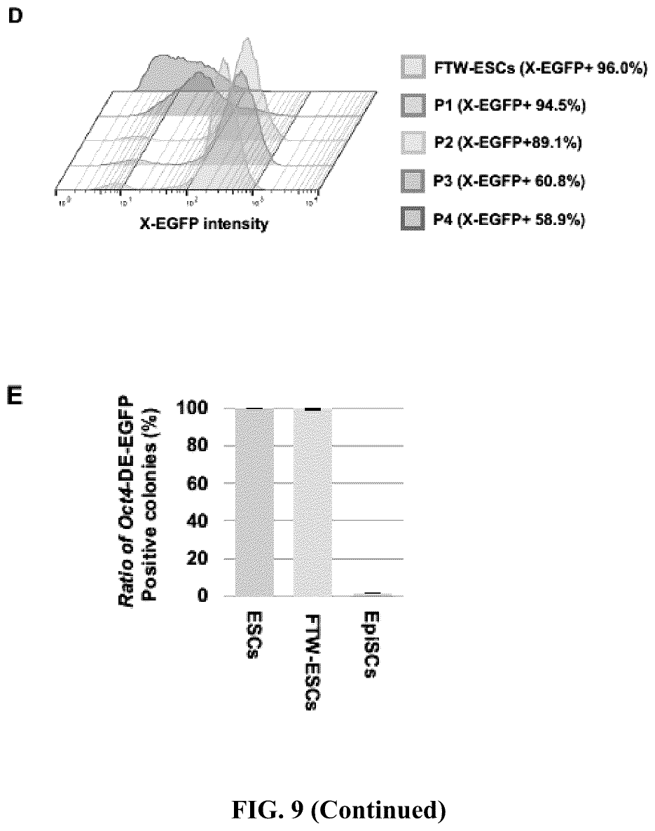

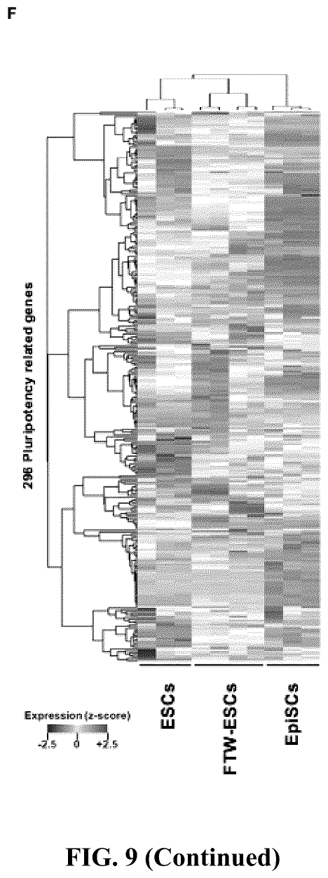

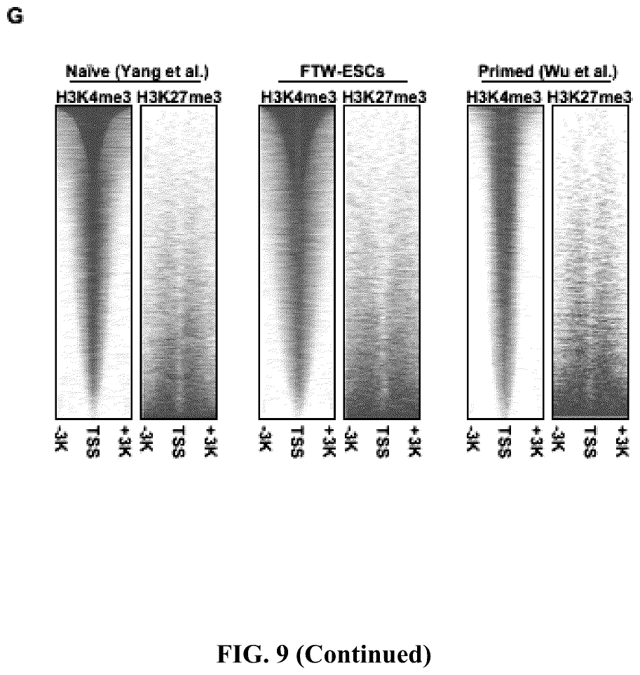

In addition to de novo derivation, a method that allowed for the stepwise and seamless transition from naïve ESCs to FTW-ESCs, and from FTW-ESCs to primed EpiSCs was developed. This was accomplished by media changes from 2iL (2i: CHIR99021 and PD0325901, and leukemia inhibitory factor, or LIF) to FTW medium, and to FR medium (FGF2 and IWR1, a canonical WNT antagonist) (FIG. 9A).

Taken together, these results demonstrate generation of intermediate PSCs from mouse blastocysts.

### Example 2. Mouse FTW-ESCs Reside in an Intermediate State Between Naïve and Primed Pluripotency

Similar to naïve ESCs, Xist RNA FISH analysis showed that de novo derived FTW-ESCs had two active X chromosomes (XaXa) in female cell lines (FIG. 1G). To quantify the percentage of XaXa cells in FTW-ESCs, an X-EGFP reporter ESC line was converted to FTW-ESCs, which contains an EGFP transgene integrated in the paternal X chromosome (Bao et al., Cell Research, 28, 22-34, 2009). Fluorescence-activated cell sorting (FACS) analysis revealed that more than 99% of X-EGFP FTW-ESCs were EGFP positive (FIG. 1I). Next, FTW-ESCs were converted to primed EpiSCs and X-chromosome inactivation (XCI) status during the transition was examined. Results showed that the percentage of cells containing Xist FISH signal dramatically increased from passage 2 (5.1%) to passage 4 (95.5%) (FIG. 1H and FIG. 9B), indicating XCI mainly occurred during this period. In addition, the percentage of EGFP+ cells decreased from passage 2 (89.1%) to passage 4 (58.9%) (FIG. 9C and FIG. 9D), suggesting that XCI is random during the transition. On the other hand, similar to primed EpiSCs, FTW-ESCs formed tight junctions (FIG. 1J) and expressed higher levels of primed pluripotency and tight-junction related genes when compared to naïve ESCs (FIG. 1K and FIG. 8C). These results demonstrate that mouse FTW-ESCs share features of both naïve ESCs and primed EpiSCs.

Two Oct4 enhancers, distal (DE) and proximal (PE), differentially control Oct4 expression in naïve and primed pluripotent states, respectively. An Oct4-DE-EGFP mouse ESC line was converted to FTW-ESCs and EpiSCs by culture adaptations. The majority of Oct4-DE-EGFP FTW-ESCs were EGFP positive, but the average EGFP signal intensity was lower than in naïve ESCs (FIG. 1L, FIG. 1M and FIG. 9E). In contrast, Oct4-DE-EGFP EpiSCs converted from FTW-ESCs were EGFP negative (FIG. 1L, FIG. 1M and FIG. 9E).

Next, global transcriptional analysis was performed using two independent FTW-ESC lines via RNA-sequencing (RNA-seq), and results were compared with published mouse ESC and EpiSC datasets (Bao et al., Cell Research, 28, 22-34, 2018; Buecker et al., Cell Stem Cell, 14, 838-853, 2014; Wu et al., Nature, 521, 316-321, 2015). Principle component analysis (PCA) showed that FTW-ESCs clustered tightly as a group separate from both naïve ESCs and primed EpiSCs, indicating that FTW-ESCs acquired a distinct transcriptome profile (FIG. 1N). Analysis of pluripotency specific genes (Müller et al., Nature, 455, 401-405, 2008) confirmed that FTW-ESCs also exhibited a pluripotency signature distinct from ESCs and EpiSCs (FIG. 9F). The global deposition of histone 3 lysine 4 trimethylation (H3K4me3) and histone 3 lysine 27 trimethylation (H3K27me3) in FTW-ESCs was examined using a low-input ChIP-sequencing (ChIP-seq) protocol (Bogliotti et al., Proceedings of the National Academy of Sciences, 115, 2090-2095, 2018), and then compared with published ESCs and EpiSCs datasets (Wu et al., Nature, 521, 316-321, 2015; P. Yang et al., Cell Systems, 8, 427-445.e10, 2019). Data analysis revealed that in FTW-ESCs both H3K4me3 and H3K27me3 peaks were wider around the transcription start site (TSS) than in ESCs and EpiSCs (FIG. 9G).

Collectively, these results demonstrate that FTW-ESCs exist in a distinct pluripotency state putatively intermediate of naïve and primed pluripotency.

### Example 3. Mouse FTW-ESCs Share Common and Distinct Molecular Features of Formative Pluripotency

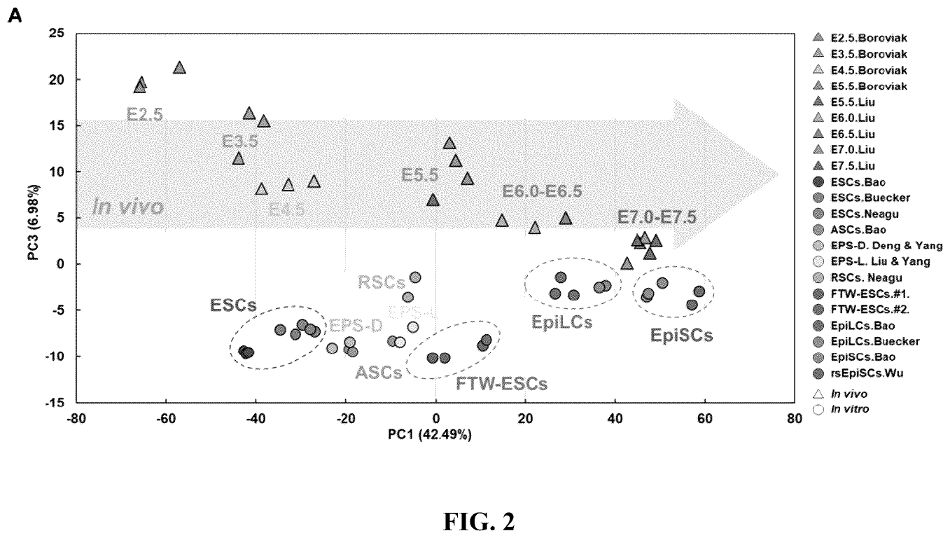

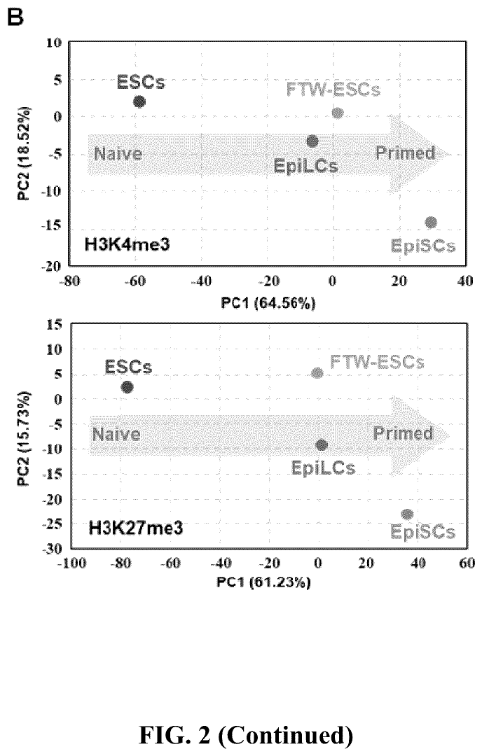

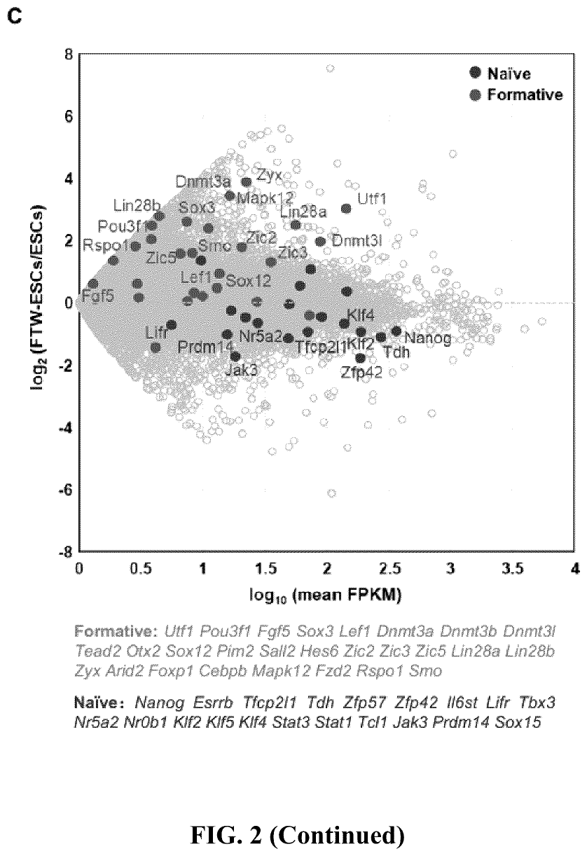

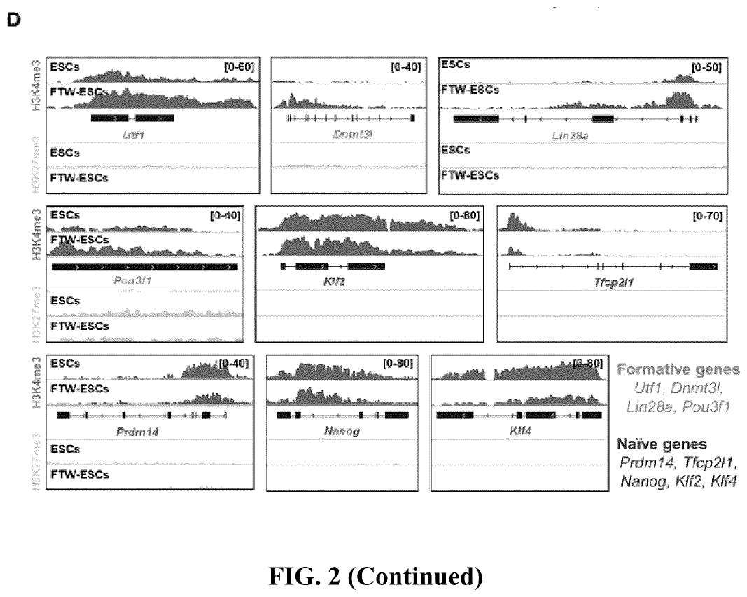

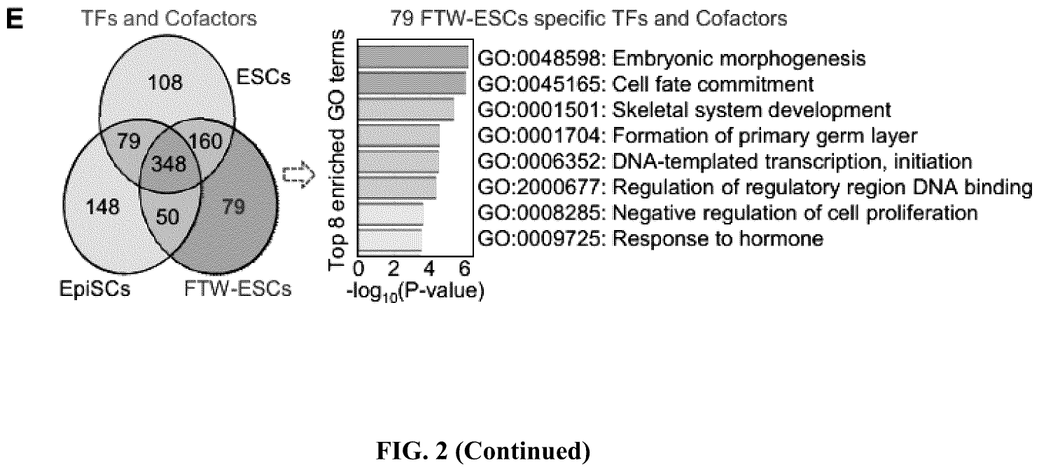

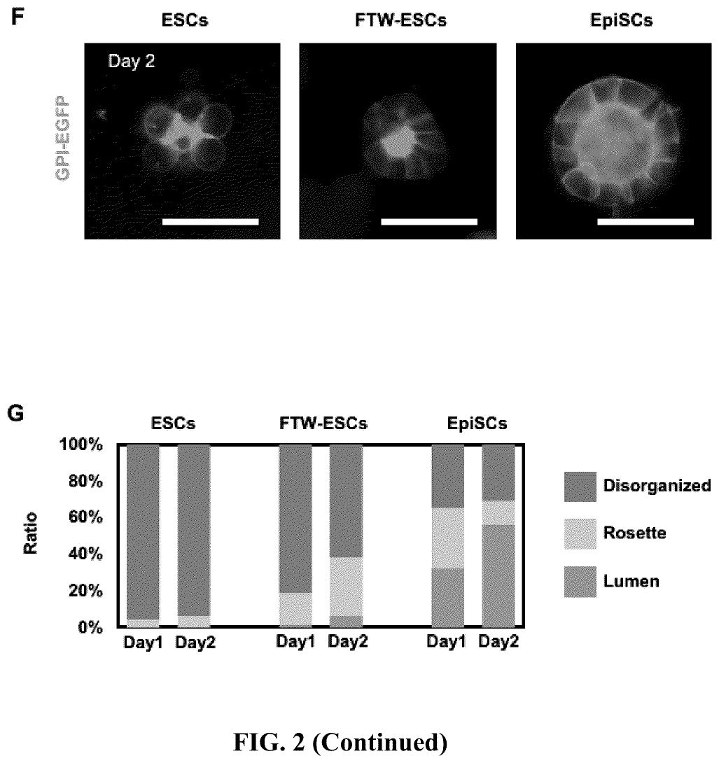

A “formative phase” was recently proposed to exist between naïve and primed pluripotency phases in vivo (Smith, 2017). To examine whether FTW-ESCs harbor features of formative pluripotency, FTW-ESCs RNA-seq and ChIP-seq datasets were compared with published datasets from in vivo (E5.5 and E6.5) and in vitro (EpiLCs) formative cells, among other samples (Bao et al., Cell Research, 28, 22-34, 2018; Boroviak et al., Developmental Cell, 35, 366-382, 2014; Boroviak et al., Nature Cell Biology, 16, 516-528, 2015; Buecker et al., 2014; Du et al., Cell Stem Cell, 22, 851-864, 2018; Neagu et al., Nature Cell Biology, 22, 534-545, 2020; Wu et al., Cell, 168, 473-486, 2015; J. Yang et al., Nature, 292, 154, 2017; P. Yang et al., Cell Systems, 8, 427-445, 2019; Y. Yang et al., Cell, 169, 243-257.e25, 2017). PCA analysis using RNA-seq datasets placed FTW-ESCs closer to E5-6 epiblasts than E4.5 and E7 epiblasts (FIG. 2A). In agreement, global H3K4me3 and H3K27me3 profiles of FTW-ESCs also clustered closer to formative EpiLCs than to ESCs or EpiSCs (FIG. 2B). It was also found that most formative pluripotency related genes were upregulated while naïve pluripotency related genes were downregulated in FTW-ESCs when compared to ESCs (FIG. 2C) (Peng et al., Nature, 144, 1-5, 2019). Additionally, H3K4me3 and H3K27me3 signals around the TSS of selected formative genes showed higher and lower levels, respectively, in FTW-ESCs than in ESCs. In contrast, H3K4me3 and H3K27me3 levels around the TSS of naïve genes exhibited the opposite trend (FIG. 2D). Moreover, from RNA-seq analysis, 79 significantly upregulated transcription factors and co-factors in FTW-ESCs were identified when compared to ESCs, EpiSCs and MEFs, which were enriched in gene ontology (GO) terms related to embryonic morphogenesis, cell fate commitment, and formation of primary germ layers, among others (FIG. 2E). These genes included: Lgals9 (NCBI Gene ID 16859), Zscan4f (NCBI Gene ID 665902), Zscan4c (NCBI Gene ID 245109), Taf71 (NCBI Gene ID 74469), Zscan4b (NCBI Gene ID 665780), Mael (NCBI Gene ID 98558), Mef2b (NCBI Gene ID 17259), Hoxa1 (NCBI Gene ID 15394), Etv4 (NCBI Gene ID 18612), Lox12 (NCBI Gene ID 94352), Esx 1 (NCBI Gene ID 13984), Plek (NCBI Gene ID 56193), Irf7 (NCBI Gene ID 54123), Tesc (NCBI Gene ID 57816), Zscan4d (NCBI Gene ID 545913), Olig 1 (NCBI Gene ID 50914), Dmrtb1 (NCBI Gene ID 56296), Hnf1a (NCBI Gene ID 21405), Bhlhe40 (NCBI Gene ID 20893), Msx2 (NCBI Gene ID 17702), Notch4 (NCBI Gene ID 18132), Tgfb1 (NCBI Gene ID 21803), Stat4 (NCBI Gene ID 20849), Csrnp1 (NCBI Gene ID 215418), Srebf2 (NCBI Gene ID 20788), Gata4 (NCBI Gene ID 14463), Hc1s1 (NCBI Gene ID 15163), Csrnp3 (NCBI Gene ID 77771), Fam83g (NCBI Gene ID 69640), Bc16b (NCBI Gene ID 12029), Arnt1 (NCBI Gene ID 11865), O1ig2 (NCBI Gene ID 50913), Uri1 (NCBI Gene ID 19777), Actn2 (NCBI Gene ID 11472), Hoxa3 (NCBI Gene ID 15400), Zfp738 (NCBI Gene ID 408068), Ankrd1 (NCBI Gene ID 107765), Arid3a (NCBI Gene ID 13496), Sox7 (NCBI Gene ID 20680), Cebpa (NCBI Gene ID 12606), Hoxa5 (NCBI Gene ID 15402), Gtf2e1 (NCBI Gene ID 74197), Arnt (NCBI Gene ID 11863), Skil (NCBI Gene ID 20482), Cbl (NCBI Gene ID 12402), Zfp72 (NCBI Gene ID 238722), Pias2 (NCBI Gene ID 17344), Hand1 (NCBI Gene ID 15110), Prmt5 (NCBI Gene ID 27374), Sap18 (NCBI Gene ID 20220), Eya3 (NCBI Gene ID 14050), Zfp617 (NCBI Gene ID 170938), Zfp866 (NCBI Gene ID 330788), Nelfa (NCBI Gene ID 24116), Nfx1 (NCBI Gene ID 74164), Slc25a15 (NCBI Gene ID 18408), Zfp933 (NCBI Gene ID 242747), Tfdp2 (NCBI Gene ID 211586), Itch (NCBI Gene ID 16396), Nkx2-9 (NCBI Gene ID 18094), Zfp941 (NCBI Gene ID 407812), Zfp87 (NCBI Gene ID 170763), Sox1 (NCBI Gene ID 20664), Irf6 (NCBI Gene ID 54139), Rtf1 (NCBI Gene ID 76246), Dmrt3 (NCBI Gene ID 240590), Recq15 (NCBI Gene ID 170472), Zfp759 (NCBI Gene ID 268670), E212 (NCBI Gene ID 242705), Pura (NCBI Gene ID 19290), Txlng (NCBI Gene ID 353170), Tpr (NCBI Gene ID 108989), Hoxb2 (NCBI Gene ID 103889), Irf8 (NCBI Gene ID 15900), Hoxa7 (NCBI Gene ID 15404), Zzz3 (NCBI Gene ID 108946), Casz1 (NCBI Gene ID 69743), Adnp (NCBI Gene ID 11538), and Xrcc6 (NCBI Gene ID 14375).

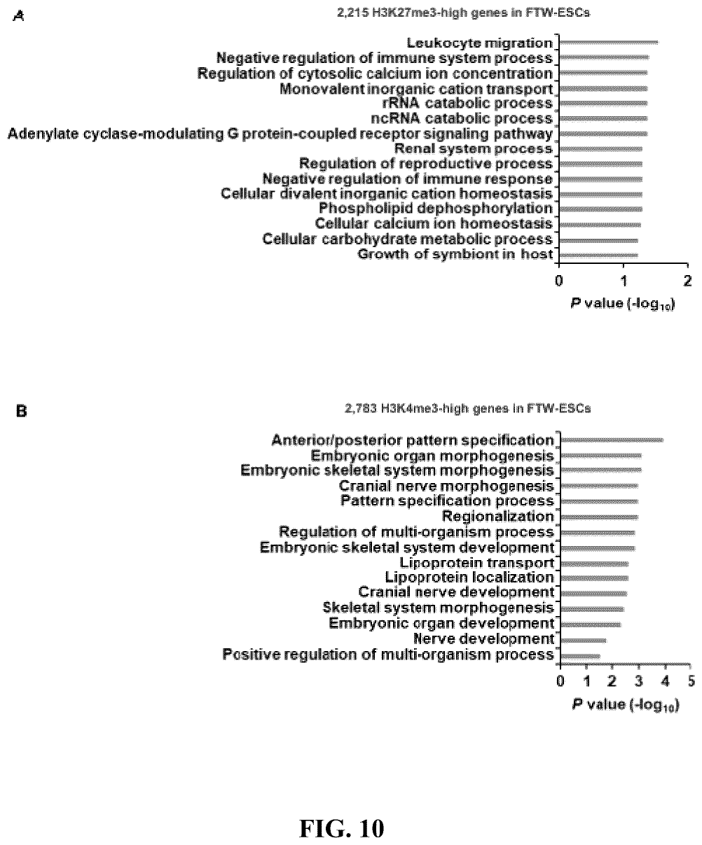

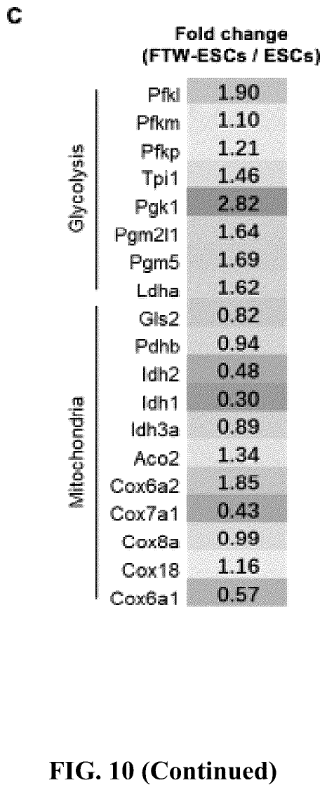

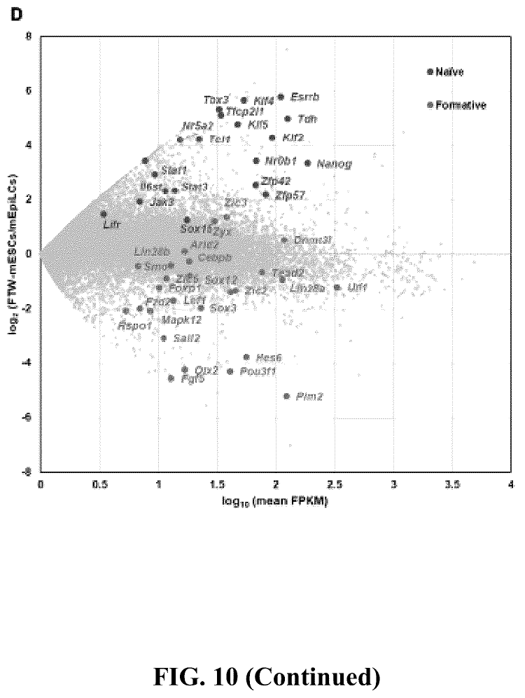

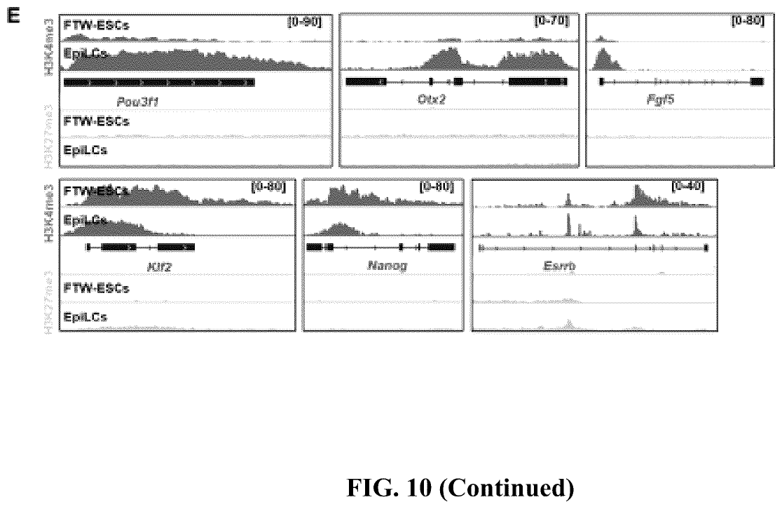

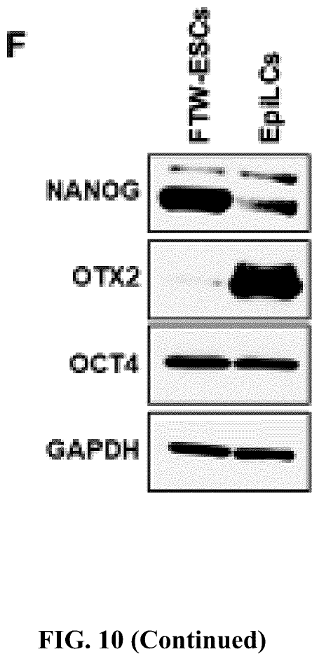

Further, through comparison with ESCs and EpiSCs, 2,783 H3K4me3-high and 2,215 H3K27me3-high genes were also identified in FTW-ESCs ( ). GO analysis of the 2,783 H3K4me3-high genes revealed several terms, e.g., anterior/posterior pattern specification and embryonic organ morphogenesis, that were overrepresented in FTW-ESCs. On the other hand, GO terms enriched in the 2,215 H3K27me3-high genes include leukocyte migration and negative regulation of immune system process, among others (FIG. 10A and FIG. 10B). For ease of reference, the H3K4me3-high and H3K27me3-high genes are listed herein below.

H3K4me3-high genes in mouse FTW-ESCs:: DLX4, MYH11, BANF2, TNFRSF25, MKLN1, CISD3, PLEKHG3, MYOD1, DOK2, BORA, MCMI, KLC4, DUOX2, NYAP2, TRMO, IL13, KRIT1, SMIM23, BEAN1, DIAPH1, NDOR1, NUDT8, TMEM234, CRYAA, NCOA3, TMEM138, PLPP3, GIPC3, NFIC, FEZF1, ABHD14B, NMUR2, KLHDC8B, TEX37, B3GNT3, TPBGL, NXPH2, PRPF40B, RPS27A, EEF1AKMT1, USH1G, NLGN1, MEDAG, SLC25A47, KCNJ4, CBLN3, CARD19, PLVAP, ERICH6, CEP95, ASB4, COL18A1, HIRIP3, PLPPR2, SPCS2, KLHDC4, KRCC1, CFAP100, NR2E1, SLITRK1, LAMPS, RPA1, BRAP, ZAR1, GLRA2, SLC39A9, CTLA4, PARP3, RPS21, CNPY4, RPS16, ARV1, ELOVL6, RPUSD4, FSHB, TOPORS, CCDC185, RNF167, JMJD7, PAX6, POLDIP2, SHH, FES, EVX2, SUN2, ADGRL4, TTC17, PLPPR4, MORN1, GRM7, KCNK3, NXPH4, GRIK1, AIRE, HTR1F, SMCR8, COX4I1, SCUBE3, ATP2C2, REEP3, NPY5R, SPATA4, STK19, FLCN, TMEM94, MRPL13, SBDS, ATOH8, TMEM154, UBD, TLE6, TMEM43, TNFRSF11B, EHD2, ARHGEF19, PTK7, AKAIN1, GSE1, A1CF, AHSA1, PAPPA2, PSD, TFAP2A, ADAM7, AP2B1, SASS6, CBLN1, TAF4, HCST, RGS1, ASB18, CKAP2L, WFIKKN1, FBXO9, NEUROG3, ARMC6, CEBPZOS, ENO3, SLC25A32, LRRC26, SLC17A9, AAAS, OVOL1, PUS3, SLCO4C1, JARID2, ZRANB2, SYTL1, TRIAP1, TMEM198, HOXA10, KCNIP4, BRICD5, MYOZ3, RETSAT, KLHL1, NMUR1, RIDA, VSIR, MSI2, AHDC1, IL12RB2, DIAPH3, LZIC, ENOPH1, GDPGP1, TMEM263, NEK8, NOXO1, SEC61B, CYP26A1, IKZF4, ANGPT4, PKLR, GALNT6, BCL2L12, TM6SF2, SLC22A7, TMEM196, KRTAP11-1, SON, MMADHC, A1BG, SGCG, CHAD, LIG3, WDR54, ZMYND15, NEUROG1, BMT2, HPF1, SEMA4A, GPR88, CWC15, GPD1, MARK3, MPST, PROSER1, CBG, PLPPR1, TNFSF13, USP10, RXFP3, KCNN4, KLHL35, ADAM33, KCNK15, CCDC70, NSRP1, DTX3, CFAP206, NIPSNAP3A, TET1, SMAD7, NOTO, ALG3, SOGAT, B4GAT1, GRP, ATF5, IL34, LAMC3, LAMB2, ACP7, DYNLRB2, MSMB, COG4, KMT5A, GPANK1, HDAC5, NPB, IFITM5, PLPPR3, DUOX1, OXT, GP9, TTC19, MYOZ1, ARPP21, KCNG4, SREBF2, HOXC12, SYS1, ANKRD17, GPR101, NCAN, BHLHA15, SIM2, SLC2A5, PTH, CCDC191, NRN1L, CTDSPL2, NKX1-1, FGD2, TM9SF2, NROB1, EAF1, KCNK9, LEP, VPS50, MFSD14B, PAPLN, ARSA, MFSD4A, RBM8A, LMO3, COPE, PRPH, CNTN6, DCAF17, NAALADL2, MEIOC, CTDNEP1, GGN, TFAP2D, S1PR2, ZIC1, SMC6, SPATA5, STX1B, CTNS, ARMT1, NCBP3, PRICKLE4, DES, TRADD, SNRPA, HSPA1A, PLPP2, SLC32A1, RHBG, PTGIR, RACK1, COL8A1, JHY, TNS2, ALX1, ERBB4, ACTN4, DDIAS, MIS12, KIF1C, CYP27B1, SDCBP2, ANO7, RAB27A, NECTIN1, HOXD3, ERICH4, TACR2, PLPPR5, CLMP, KCNIP1, MYH7B, CENPL, NCAM2, COPS3, LRG1, RAB35, NKX2-3, DRD1, EMG1, HPS5, SORCS3, YIPF3, IL12A, RUBCN, CRLF1, IYD, PDE6B, HOXB3, CHODL, CFAP45, MED5, WDR83, DIAPH2, BARHL1, NKX2-1, MROH6, LCT, DAND5, UNCX, INHBC, APOBR, CRELD2, MMRN1, SIRT7, HOXB1, KCNRG, SDR39U1, OLFM3, PPIL4, TMEM54, KARS, CCDC80, CRCP, OXNAD1, SRRD, ARMC5, CREB3L3, MNX1, TUBE1, MOB1A, GNA13, TMEM40, VAX1, MRPS26, LENG8, F11R, GRIA2, SNAP47, SURF2, PIPDX, PLPP4, PSMA4, IL12B, DND1, ANKUB1, ABTB1, QRFPR, NOC2L, CELSR2, DPT, APBBlIP, AUNIP, NAA50, DCUN1D5, SIX3, SLC35F6, PRMT9, CLCN4, HOXA3, MATK, LRSAM1, RNMT, RPL13, PXN, STUM, USP20, PLPP1, LRRC4C, POLH, PCP2, IMPG1, EPHA8, EREG, SLC13A4, VSIG10L, SVBP, CRACR2B, AGR3, P2RY2, FOXS1, P2RX3, EMSY, DNASE1L3, DNAAF5, ANKRD33, PTGDR, CCER1, KCNJ5, MTNR1B, GNB2, TFAP2B, PNPLA7, RIC8A, TRPC4, CRP, CILP2, NDUFS3, IDUA, ZCCHC13, TTI2, MFSD14A, SLC38A11, DLX5, QRFP, RAMP2, PTPA, PPP1R27, LING02, CMC4, GLRA3, MUTYH, ABHD15, MRPL14, MRPL49, TFPT, FAM91A1, HAND1, WDR46, SLC34A2, NECTIN3, KMT2B, EMILIN1, DONSON, NEPRO, ATP1A4, KRTCAP2, ZBTB32, DDX28, LMOD1, PIP4K2B, TMEM253, PTGFR, CFAP77, TRIM15, KAT7, SLITRK6, HSPG2, RBKS, GPR149, RING1, NCBP2, EFL1, KCNA5, MST1, HIC2, PRR16, HOXC10, ITGB2, PAQR6, HRH1, LY86, APPBP2, LIPE, CD151, CLEC3B, DPP10, BARX1, SHISA8, ITGB7, ARGLU1, MYL10, CDK9, ADAM18, SNAIL PLPP7, ASPDH, DIRAS1, GABRB1, EPS8L2, MUC1, PLEKHD1, OLFM4, GSX1, STRN3, CNGA2, SIDT2, RNF40, MRPL21, WNT7B, TACR1, VIPR2, SEMA3B, RNF8, PEBP4, DBX2, DPM1, GFY, KYAT3, FAM162A, SP3, IKZF3, MRPS24, HNRNPA1, PCNX4, PON1, GRIN3B, KCTD10, TTC6, A3GALT2, OCSTAMP, LYL1, AMD1, RNASE12, GLDN, CCDC189, TNNI3, SLC22A12, DHX38, SLC6A12, RAMP3, PRLHR, GPSM3, RPL23A, PLAGL2, RHBDL1, AQP4, TNFSF9, CNTN4, SLC46A2, ARTS, GABRA2, ECM1, GANC, NANOS3, CCDC137, SPRY4, CHKB, SQSTM1, ASCC1, ESRP2, SAMD11, LSM3, TRIM65, IFNK, LNP1, FCF1, GJD4, FREM3, TECRL, LRRTM4, KCNH7, SPPL2B, CHST9, OLIG2, KCNG2, TMEM200C, ZDHHC22, MGME1, NECTIN2, CCHCR1, PRPF4, GSTP1, MIR509, 7530416G11RIK, OLFR1270, MIR652, VMN2R66, GM4981, PRSS44, GM6634, TAGLN, GM35206, 9530082P21RIK, MIR7060, AY702102, 1700022H01RIK, B230311B06RIK, PAKAP, 4930415L06RIK, 4930567J20RIK, GNA15, TMEM15000S, MIR124A-1HG, C230029F24RIK, VMN2R86, 1700030NO3RIK, 4921525009RIK, MIR5120, MIR582, CD93, SNORD92, GM37013, RPAP2, CCR7, GM5478, PRL2C1, HSF1, 1700128A07RIK, GM20063, MIR6976, GDFS, SLAIN10S, ZSCAN29, KYAT1, PCDHGB1, MIR6990, 6030466F02RIK, OLFR907, GM20765, GM12128, GOSR2, 4930471M09RIK, OLFR1170, GM6213, LRRC57, RBP3, KIR3DL1, VMN1R129, MRGPRA2B, DNAJB8, ZC3H12D, SLC22A26, GM21671, GM4489, E130218103RIK, E2F8, HOTTIP, MIR181D, LAT2, 1700008C04RIK, VEGFD, MEI1, F830045P16RIK, PINC, 4933408J17RIK, MIR6991, ADAM26B, MIR680-2, SSXB6, SAP3OBP, 5730488B01RIK, 4930440C22RIK, GM15517, OLFR771, OLFR1314, 1700001DO1RIK, GUCA1B, CCKAR, FPR-RS3, MIR193A, GM3604, POP1, DEFB41, OLFR1095, TRIM12A, RNF151, COL4A3, AI463170, RNF31, CCDC33, NLRP1C-PS, 4933432G23RIK, 1700110K17RIK, FBXW14, MIR5617, TIMD4, 1700026J14RIK, OLFR8, HOTAIR, FNDC7, MAN1B1, PRR32, GM15679, GM12374, ZWILCH, 4930544D05RIK, TCAP, NAA60, GM906, GM32511, MSR1, OLFR1344, MIR6941, RASGRP4, FBXW28, ZDHHC3, RAVER1, CTS7, SULT1C2, MIR3547, ASTX6, 4932413F04RIK, MIR8100, GM17767, PLPP5, SNORA43, VMN2R92, MIR6911, MIR8103, ZFP972, FTSJ3, MIR6988, GM30173, KIF4-PS, NPCD, MIR5106, 5930438M14RIK, LMNTD2, 2410021H03RIK, ACNAT2, GM6567, BC048671, 4930563H07RIK, AU015836, D230030E09RIK, MIR615, NCAPH2, OLFR951, 1190028D05RIK, 4933428G20RIK, OLFR722, MIR6350, GM5891, ELDR, OXGR1, 4930557K07RIK, 4933400F21RIK, GM5128, 4930445N18RIK, SPATA33, MIR676, GM38426, GM19345, GM13749, DLX40S, MRPS7, FHLS, 1810041H14RIK, BC049762, FSHR, 4930442J19RIK, OLFR1495, VMN1R30, 2310069B03RIK, BVHT, GM16793, OLFR1166, CES4A, GM1715, DBPHT2, 4930558J18RIK, SLC26A8, 2300002M23RIK, TAS2R119, 1700095A21RIK, ZFP944, KISS1, E230029C05RIK, GM5114, GM5113, FITM1, TULP2, GM10354, PTGER3, GM20822, OLFR344, MIR467F, 5830473C10RIK, SCRG1, OLFR731, 4930440119R1K, BROX, GPS1, 4930503B20R1K, 2310040G24RIK, 1700099109R1K, GM14204, GM20752, 1700049E17RIK2, C87414, SNORA28, PRAM1, OLFR1390, SNORA70, 6430562015R1K, LCN4, IL18R1, 2010010A06RIK, PRSS43, 5031425F14RIK, 4930441H08RIK, SPINK4, TRIM30D, GM12159, GM32455, TESPA1, CACNG6, MIR3965, DNASE1L2, ATP6V1G2, PCDHGA6, GM13490, HOXAAS2, 9330182014RIK, GM19303, 2900040C04RIK, 6820426E19RIK, LINS1, 4933411E08RIK, 2810442N19RIK, 1700024B18RIK, TMEM38A, RHN01, 4930590L20RIK, PCDHGA3, PLATR8, 4930413E15RIK, GM36283, GM1527, 4933412006RIK, DAPL1, MC3R, TMPRSS11F, 2810433D01RIK, MIR204, 1700034K08RIK, 1700036G14RIK, PCDHB5, OASL2, GM10248, GM10687, 4930471G24RIK, CATSPERB, GM10710, GM4861, OPALIN, MIR465, 1700027H10RIK, SLC5A2, GM20815, OLFR94, CYP2D9, RAB20, KRT222, MIR125B-2, H2-M1, DI02, TNN, PDZD11, PCDHA5, GM3636, AKP3, SLCO6D1, BC048679, MIR5127, MIR28B, MIR6418, MIR7089, MIR706, GM4788, GM20597, GM5091, GM20172, PSMB11, GM5535, MIR3082, GM5591, VMN2R-PS11, PANTR1, SIGLEC15, PTMS, GM12238, 1700022E09RIK, SERPINI2, MIR5121, P2RY10, GM5415, URAH, ERP27, MIR5104, GM10635, 4930443020RIK, ZBTB49, GPR151, SERPINB3B, GM6994, MIR7241, 4933403008RIK, H2AFB1, TBPL2, GM12695, MIR6902, GM23450, BTNL10, GM27740, 1700003L19RIK, 1700065L07RIK, D16ERTD519E, FBXW20, CYP4A12A, DRC7, OLFR1271, HILS1, TRPC2, OLFR1257, 4930556N09RIK, LRRC10, 4930431P22RIK, E230013L22RIK, MIR1892, 9630028H03RIK, MIR3108, OLFR910, SERPINB9F, DNMT3L, BCL2A1B, GM536, 1700007F19RIK, 1700028D13RIK, SELPLG, MIR3106, GM7788, RXFP1, C1QTNF3, ZMYND10, AI606473, UBAS, ITIH5L-PS, 1700009N14RIK, GM2109, 4930478L05RIK, CYPT2, GM15343, GM31938, GM17597, GM4651, HAVCR1, PLATR7, SPN, PPP1R2-PS9, RNF113A1, USPS, ESP1, A930009A15RIK, 4930413G21RIK, APCS, OBOXS, RBAKDN, 2900092C05RIK, 1700044K03RIK, 2310039L15RIK, 4921518K17RIK, RHOX3G, 1700092C10RIK, PCDHGA9, 4930548J01RIK, CPXCR1, OLFR596, ZFP280B, PRSS42, SNORD69, RP1, MIR7J, 1700022A22RIK, 1700095J03RIK, OLFR1507, CSTF1, GM20594, G630064G18RIK, 9530020112RIK, KLHDC7B, MIR767, 1700003D09RIK, CD209B, SLC22A22, 4930593A02RIK, 6330415B21RIK, ARHGAP35, 4930417022R1K, NAT8F1, FSBP, PCDHA9, 4930467K11RIK, MIR466Q, GM867, BYSL, ATXN7L1OS2, 3110009F21RIK, ACTL9, BB031773, GM33727, 1700027A15RIK, MIR326, SNHG15, 1700052122RIK, ACKR1, 4932415M13RIK, G6PD2, KBTBD13, NOSIP, 9130221H12RIK, GM4793, GM13315, SMIM9, MIR6420, GM3942, MIR343, 8430430B14RIK, GM10440, NOS3, OLFR513, 4930579F01RIK, 1700040D17RIK, SPEER4C, 1700009C05RIK, GM26684, AI839979, TLR9, OLFR933, H2-T3, GM4956, PLAC9B, VMN2R112, LRRC72, GM1943, CEACAM-PS1, CRYGC, 4930526L06RIK, 4833412C05RIK, 1110002J07RIK, 4930431P03RIK, UBL5, GM10471, HSD3B3, SDHD, MIR490, FYB, UPK1A, MIR671, NUDT16L1, 8430436N08RIK, 4933433H22RIK, 4930592A05RIK, TREX1, GM15908, AMTN, AI197445, 4930554G24RIK, VMN2R65, SPRTN, COX11, GM32865, MIR1298, IQCJ, GM14207, BLOC1S6OS, 1700025B11RIK, SLC35G3, GM20806, GM26633, SOX6OS, GM10377, RGS18, SORBS2OS, ZFP488, MIR7088, GM15569, TCAM1, MIR3968, 4930404H11RIK, CYP3A44, MIR5046, CCK, PCDHB10, 1700018L02RIK, GM5640, GM17733, PRKDC, MIR692-2, MIR1966, AMY2A5, KHDC3, TREM1, D6ERTD474E, GM4301, TAS2R114, MIR3961, MIR7232, AMELX, OLFR339, GM10532, LNCPINT, OLFR553, SCGB2B12, E230016M11RIK, GM26710, GM21190, 1700109G15RIK, MAGEF1, PTX3, MIR1963, GM38404, CLDN34B4, MIR28C, MIR7011, DEFB33, NTS, C330024D21RIK, CHIL5, OXCT2B, C730002L08RIK, CYP3A25, 4930505N22RIK, BC042761, SPATA31D1C, MIR141, NXPE2, ZMATS, NOL3, ADAM26A, SKINT6, GM14743, VILl, GM11190, MYCS, GM10823, GFAP, CYP2C53-PS, GM26994, HALR1, TINAG, ZC3H11A, MIR6995, FAM166B, ENPEP, MIR3069, AI450353, BCL2L10, FBXW27, EPN3, 2810408A11RIK, GM30853, SMIM22, 4933427E11RIK, MIR5098, GM4750, GM10466, CLDN22, LOC102632231, 4930548K13RIK, 0610005C13RIK, RNF224, TNXB, OLFR924, GM17634, MIR7070, VMN1R2, 6430553K19RIK, TERB1, MIR3473D, MIR448, OLFR796, MIR5118, MIR144, BCL2A1C, MIR2139, 4931420L22RIK, TJP3, AU022793, MIR6395, ALMS1-PS2, GM15934, OFCC1, ATAD3AOS, 4933417G07RIK, GM13547, EHBP1L1, SOD3, OSGIN1, GM35496, SNHG14, 4930448118RIK, ZFP973, GM41410, GM20219, PCDHB2, OLFR1312, VMN2R57, BSN, GM5523, 4930525G20RIK, OLFR46, IL17C, TAS1R2, MIR8120, OLFR855, GRXCR1, A630023P12RIK, 1700109K24RIK, GM20767, MIR7213, 4932435022R1K, 1700061J23RIK, 6430628N08R1K, 4930432J09RIK, RNF208, 1810007C17RIK, 2610020C07RIK, CXCL13, VMN1R59, GUCY1B2, ACTL10, GM7714, CLEC4A2, OLFR16, GM10681, CDK11B, CYP2C38, MIOX, SNORD19, GM14496, CRCT1, MIR6973B, KRT17, TAS2R110, SNHG18, CXCL3, IFI204, MCAM, RD3, XNTRPC, 1700030A11RIK, GM14486, OLFR1320, C1QA, VMN1R72, MROH3, VMN1R40, GM31763, GM14548, FCMR, MNDAL, A930012L18RIK, PADI4, 4933440J02RIK, 4921504A21RIK, OLFR362, 1700003G18RIK, FUT7, GM36595, MIR7058, PRR30, CTRL, PCSK2OS2, GM29669, GM6194, 4933400L20RIK, MIRT1, PCDHGC5, MIR7018, ZFP965, MFSD4B5, CPNE6, MIR7047, CYP2J8, PRR3, CLEC4B1, MIR6237, SNORD85, VMN1R226, MIR7029, SYCE1L, HPN, MIR1187, NPPB, MIR1192, 1110015018RIK, TMEM181C-PS, GM15694, GM12789, OLFR142, AMY2A3, KCNJ14, GM26641, VMN2R62, CD226, RNF138RT1, LRIT3, STOML3, HSFY2, 9230114K14RIK, GM9961, 4933401B06RIK, OLFR1324, GM12532, GM6602, 4930459L07RIK, ZFP970, MIR153, MFSD4B1, 1600020E01RIK, KRT12, LRRC3C, OLFR1504, LOCKD, KRT77, 1700008K24RIK, PCDHGA7, VMN1R29, HOXB8, MIR684-1, VMN2R118, 4921511C20RIK, SPAG6L, MIR142, SNORD118, GM20756, OLFR781, PCDHGA5, 4930557F10RIK, MIR6370, SLY, 1700123012RIK, OLFR461, CLDN34D, 4930559C10RIK, GM5592, PCDHGB7, IFI47, COLEC11, CLDN34B3, CLDN17, 5033406009RIK, MIR205, ACTRT2, SLC22A19, CTCF, B230319C09RIK, TMPRSS9, ZFP971, MIR8094, GM14781, SOX10, 4930523C07RIK, MIR6948, GM6578, PABPN1L, GM3716, MIR195B, 4930528P14RIK, ECI3, GM32926, PRR22, PCDHGB5, GM5346, GM38560, 1700027F09RIK, 9330158H04RIK, UCP3, PPP1R2-PS3, 1700015G11RIK, PRSS2, OLFR59, TLR12, A530021J07RIK, MIR678, MIR7054, MIR710, GM30731, POU3F3, 1700019M22RIK, 4930451G09RIK, GM12354, TAS2R134, SNORA41, GPR165, GM16617, B430010I23RIK, 4930558K02RIK, ARHGAP30, GM14525, GM4971, MIR7K, 4930511A02RIK, 2810404M03RIK, GM3428, PLATR22, SPRN, PTF1AOS, CTSJ, SNORD52, GM17308, ZFP423, AVIL, MIR7094-1, KIF2B, 1700006H21RIK, POTEG, MIR6906, PCDHGB2, 4833428L15RIK, RAD21L, GM8096, 9330198I05RIK, VMN1R73, A330032B11RIK, GM9530, GM11917, GM38438, C530044C16RIK, CACNG2, MIR6239, MIR367, MIR6342, LCE1M, MSH4, GM31520, 6330415G19RIK, 2810004N23RIK, OLFR449, D130009I18RIK, PBRM1, MIR592, 4930550C17RIK, 1700025C18RIK, ESP31, 4930598F16RIK, MIR129-1, PGLYRP2, GZME, KLK1B16, MOCS3, VMN1R-PS103, HOXA110S, GM41279, TMCO5, HTRA2, D530049I02RIK, E130304I02RIK, D030025E07RIK, FCGR1, GM5907, RXFP4, ERI2, 4931440J10RIK, SCGB2B11, RBP1, MIR1930, 4930563M21RIK, GM29684, GM3143, DOK3, ZFP572, OLFR593, A430089I19RIK, 2700099C18RIK, MIR7663, APOBEC4, DNAH10, 4930519G04RIK, GM10640, GPBP1L1, 4930544G11RIK, F9, GM10745, EBI3, CCR4, SAP18B, FGFBP1, MED9, DEFB38, C5AR2, PLATR30, 4930474G06RIK, MIR6362, OLFR39, A130030D18RIK, 4930412013RIK, PANTR2, 1700045H11RIK, 6430710C18RIK, CALHM1, MIR5108, SCGB1B3, GM6455, MDRL, OLFR161, GNB3, FAHD1, MIR7017, 1700016G22RIK, FUT4-PS1, MIR879, OLFR585, GM16731, SDF4, 2210418010RIK, GM15997, BRIP1OS, INS1, CLDN7, FABP2, ASB10, IRS3, ZMYND12, IFNA9, GZMF, 2410017117RIK, RNF170, GM15133, OLFR744, CYLC2, GM6329, ZFP24, HAST, VMN1R8, 1700066J03RIK, TNFSF10, GM436, STMN1-RS1, GM19705, A630075F10RIK, 9430078K24RIK, PCDHGA2, STFA3, PBP2, PTPRV, GM21057, 1700024P04RIK, NEUROD6, 1700092E19RIK, GM13446, 1700003P14RIK, GM30790, 4930470006R1K, 7420700N18RIK, 2400006E01RIK, MIR8117, MIR7688, GM5105, IFI44, MIR6378, GM11166, KLK8, SCARLETLTR, GALNTL6, SNORD1A, OLFR635, PCDHB17, A230083G16RIK, FLG2, OLFR688, MIR1971, GM34184, TAX1BP3, 4921529L05RIK, OLFR295, SULT3A2, A530013C23RIK, 9330175E14RIK, COQ4, 1700097NO2RIK, 9530026F06RIK, ASCLS, MIR7048, SPEER3, SCGB2B27, SULT2A1, ZFP865, HAO1, ADGRE1, 1110059E24RIK, 6030471H07RIK, OLFR211, MIR3971, MOAP1, SEMASA, SLC15A5, CTF1, GM3363, TYMP, SNORD16A, 4933402J10RIK, GM597, GM34284, SNORD37, 1700126H18RIK, OLFR124, GM4297, ABI3, 2610316DO1RIK, IL17A, GM36246, FFAR1, D030068K23RIK, CGA, 5430434115RIK, GM4262, C430042M11RIK, 4930455G09R1K, 1700010DO1RIK, 4930502E09RIK, A830019P07RIK, 1700001G17RIK, 4930477N07RIK, OLFR90, DANCR, SAA2, MIR7656, GIMD1, D630033011RIK, GM32461, CYP2W1, C2CD4D, AW549542, GM33340, TNFRSF13C, SNORD22, PLA2G4B, ARHGAP33OS, BCO28528, OLFR564, C330004P14RIK, DNAH7C, SCGB2B15, GM20750, A630095N17RIK, MIR6388, CRYGF, LOC102640673, GM10058, GM6377, GM35135, MIR3964, AGTR1B, NLRP2, 4930432K21RIK, PCDHGA11, GM36669, PODNL1, PPP1R3A, GM8633, TMEM266, U90926, MIR7083, OLFR1196, 2510002D24RIK, ZNRD1AS, GM13238, D030045P18RIK, GM8909, C1QTNF1, SLC14A2, MIR6912, 4930402F11RIK, IL23A, B430212C06RIK, ALKBH3OS1, WDR53, LNCBATE1, GM26689, GM16497, XNDC1, GM5868, GM26876, MRLN, MIR22, SPANXN4, CRNDE, ZFYVE19, GM17619, BGLAP, TRIM54, FAM120AOS, PCDHB20, 1700020G17RIK, 4930539E08RIK, GM9855, 4930428E07RIK, CCDC166, 4930459C07RIK, GM14635, CYPT15, 3300002P13RIK, VMN1R62, AKR1D1, PLET1OS, SNORD57, SSMEM1, OLFR761, SULT6B1, GM19582, KCTD12, H2-T-PS, 9430021M05RIK, MIR6335, MIR6411, TXNDC8, KIR3DL2, OMP, GM41289, GM3055, 4930553E22RIK, B230344G16RIK, CCDC7B, SKINT11, SPINK10, TMEM132COS, TSSK5, MRGPRD, P2RY12, A430046D13RIK, 2200002J24RIK, MIR6367, OLFR1258, BPIFA3, CCDC92B, NNAT, BC147527, CYP2C66, REG3B, E130112N10RIK, 4930594021RIK, GM20740, OLFR38, 1700094M24RIK, PCSK2OS1, RIBC2, LYZ2, MIR8090, ZG16, GM26688, OLFR1154, MIR7647, MIR7003, VMN1R205, TRIM42, MIR6930, GZMK, GM35986, SLC22A29, KRT2, HIST1H2BP, PFN3, GM38414, CAPZA3, VMN1R17, GM15825, 4930467D21RIK, DPPA4, VMN1R101, PCDHGA10, 4930447F24RIK, MIR7229, PMCH, BORCS8, GM7978, GM29683, MFSD4B4, 4930583I09RIK, ZP4-PS, A730006G06RIK, 1700001C02RIK, GM5860, ZFP467, GM34240, A930001A20RIK, SLC52A2, HSH2D, 1700029N11RIK, MIR6923, CEP83OS, 4931408C20RIK, PCDHGB6, VMN2R61, MIR8110, GM4961, GM7607, LRRC25, 1700008P02RIK, GM30726, OBSCN, MIR6336, 4933439C10RIK, MIR1936, TMEM235, NPY6R, 2010310C07RIK, PTH2, POU1F1, CNGB3, EXOC3L4, PCDHGA4, A430035B10RIK, SLC25A41, GM12886, MIR695, DLX6OS1, GM28881, GM5142, 1700123L14RIK, OLFR419, MIR6240, VMN2R98, MIR1953, GM26851, GM38671, ACR, TEX28, POLL, ZIM3, OLFR165, HBQ1A, PCDHB7, NKPD1, MIR146, VMN2R12, GM31333, SNORD47, 3100003L05RIK, SLC39A12, GM5294, MIR763, F630206G17RIK, CYPT12, D830032E09RIK, D830005E20R1K, 2210406010R1K, LOC100862268, KRTAP21-1, 1700091H14RIK, MGAM, MIR466F-4, OLFR1505, ZFP512B, OLFR54, GM16630, MIR467H, GM16833, REEP6, 1700017L05RIK, PCDHB4, GM38425, CHIL4, ABHD18, GM13057, 2310016G11RIK, CUBN, MIR1195, BC061195, MIR145B, 4933417D19RIK, L3MBTL4, CESSA, GM4846, CSNKA2IP, GM5086, KCTD21, BIN2, HKDC1, 2410022M11RIK, MIR7215, ANKRD7, MAF1, MIR3963, MIR6373, VMN1R238, GM44505, KLRB1, PIRA2, CXCL15, 6330418K02RIK, GZMA, 1700113B09RIK, 1700012I11RIK, SSTR4, GM10400, 2310030A07RIK, ZFP967, 4933413J09RIK, GM4872, YY2, OLFR298, MIR346, C8B, KRT87, 4930448C13RIK, VMN1R82, TRP53, MIR5125, CHDH, VMN1R20, 4930430A15RIK, MIR568, TFF3, DMRT1I, LDHAL6B, VMN1R174, ATP6V1G3, GM2516, SERPINA1D, GM20939, 1700054A03RIK, GM11468, MIR7227, KRT4, SPEER9-PS1, MIR6357, SPEER2, OLFR1339, GM12018, MFAP1A, DLEU7, OLFR215, ZFP804B, PCDHB9, VMN1R38, GM33301, GM3776, MIR683-2, GM38509, 2610306M01RIK, OLFR1323, MIR3090, GM8979, GM11758, GM20036, GM23363, OLFR958, PNMT, GM16104, GLOD4, GM4632, URAD, CTS3, 1700049E22RIK, MIR133B, ZFP148, SPATA31, 4933402C06RIK, GM15555, INSL3, GM7457, 4930519K11RIK, D930016D06RIK, PCDHB13, 4732414G09R1K, REG2, 4833422C13RIK, 4921507G05R1K, MIR6355, GM19782, 4930579H20RIK, GM11681, 2900079G21RIK, MUP10, 1700012D16RIK, MIR143, GM30108, OLFR834, CMTM1, OLFR456, SLFNSO5, 4930586NO3RIK, LOC106557447, GM4251, KAP, 1700092M07RIK, PI15, ABCG8, OLFR641, GM26579, GM10324, VMN1R56, MIR182, VMN2R113, 4933412E12RIK, GM12130, RPH3A, GM19585, CFAP52, ACOT5, GM20268, GM23284, VMN1R139, PDZK1, 5730420D15RIK, MIR3057, MAMDC4, NACAD, TTI1, GM11567, 1700123J17RIK, 4930558F17RIK, OBOX1, ERDR1, MIR6412, MIR3095, 4930545H06RIK, 4930578M01RIK, MIR130C, 4930515L19RIK, ADAMTS4, GZMM, UBASH3A, PRR29, TNS4, OLFR691, OLFR322, MIR1931, MIR219A-2, MIR6368, GM1553, VMN1R184, SGF29, GM21379, 9530036011RIK, MIR9-3HG, PARD3BOS2, C330024C12RIK, MYL7, MIR1190, C030013C21RIK, GM5914, CRISP2, MIR6999, D130017NO8RIK, GM15401, CLIC3, 4930554H23RIK, ADAM34, NESPAS, MIR6925, PALMD, 1300017J02RIK, 1700012B09RIK, 9430014N10R1K, GM32172, A830029E22RIK, B130024G19RIK, ARHGAP15, VMN1R23, MIR6539, TMEM175, FXYD7, ZFPL1, LOC108167416, GM6812, OLFR65, GM11217, MRGPRB3, GM8709, NDST4, 4933438K21RIK, MAGEB5, MFRP, MIR6387, CYP3A16, MIR124-2HG, OLFR63, F13A1, TCTEX1D4, GM32141, GM14725, B230354K17RIK, 4930474N05RIK, DEFB14, MIR3078, OLFR1269, GM10768, SCGB2B26, GM10814, 4933402J15RIK, SNORD93, OLFR1135, NELL1OS, IFNA16, GM7932, 4930545E07RIK, MIR6943, GM38431, R3HDM1, 1810013A23RIK, CNTNAPSC, FMR10S, 1700086006RIK, OLFR164, OLFR773, MANNR, GM29461, AI115009, GM12191, 4632428C04RIK, 2500002B13RIK, RSC1A1, OLFR1274-P5, MIR7091, B930018H19RIK, VMN1R121, 4930428D20RIK, 2210420H20RIK, OLFR1350, A730004F24RIK, ZFP27, GM2027, MIR3472, MIR7660, GM10845, MYADML2, LYG2, 4933433F19RIK, SNORD1B, 5430402E10RIK, TERB2, 1700020D05RIK, GM7271, RPRL1, GM12925, MIR6921, GM7134, SVAL2, GM6644, 4930448F12RIK, 2310003N18RIK, 4930515G16RIK, GM26685, GM33337, CXCR3, CETN1, MIR599, CYPT1, LOC102632463, VMN1R15, MUP9, GM28535, 4933413L06RIK, CCDC183, PMFBP1, GM3336, GM15326, CYGB, MIR219B, 8030451007RIK, BC051019, 6530403H02RIK, MIR5099, 4930584F24RIK, YOD1, TAS2R139, 4930512B01RIK, PGLYRP4, 1700031L13RIK, 5033428122RIK, ALKBH4, PTGS2OS2, RNF166, ZFP750, NSMCE2, CBR2, MIR7020, CCDC146, MAU2, RAB11FIP4OS2, GM36536, DPPA2, CCDC121, EID3, GM29686, 1700031A10RIK, NUP98, GM22109, 1700054K19RIK, GM32693, GM29811, SCG2, F830208F22RIK, PAUPAR, 4930405D11RIK, A930031H19RIK, GM26760, LYZL1, GM10486, GM21814, SNX17, 1700023F02RIK, SPEER4F1, 4930502E18RIK, 9030612E09RIK, MIR6942, NUP62, MIR7230, 4930488B22RIK, SDR16C6, MIR6398, CCDC163, OLFR183, GM11827, 4930583P06RIK, GM31747, MIR147, MIR200C, GM2042, LAO1, GM15179, CCDC34OS, CES2A, OLFR332, C87198, 1700125G22RIK, RBM46OS, SELENBP2, SLC44A4, 1700109G14RIK, PLATR10, CDH5, PDZD3, GM15509, DIOL, MUP18, GM19276, 1700111N16RIK, D430036J16RIK, MIR9-3, 1700003122R1K, GM20362, 4930524C18RIK, MIR6402, 1700109108R1K, MIR7236, GM5941, GM10638, BBOX1, OLFR380, LOC105245869, AA545190, GM6345, 4930568G15RIK, LTA, MIR155HG, 3930402G23RIK, GM10548, SNTN, F630040K05RIK, 4930404A05RIK, IP6K3, ZFP366, GM4425, MIR1899, OLFR1090, GM2012, 4933427D14RIK, SPIN2-PS1, COLEC10, MUP15, MIR8099-2, MIR7023, LHX1OS, KCNJ6, SERPINA3F, GM21992, 1700030M09RIK, GM38403, 4930539M17RIK, 4930429F24RIK, VMN2R83, 1700010J16RIK, 4930558G05R1K, 4930529C04RIK, 4921513103R1K, GM11266, GM13848, AQP6, RNF222, OLFR1193, CEACAM14, MSGN1, 4921533120RIK, OLFR1118, LOC108168459, NRP, A330069E16RIK, MIR664, 3000002C10RIK, 2310010J17RIK, 4930474H20RIK, 2310020H05RIK, COG5, 4930405L22RIK, OLFR143, NPHS10S, F930015NO5RIK, MUP21, MIR6975, AU023762, KRTAP4-16, PRSS32, IZUMO1R, B020031M17RIK, 4930524B17RIK, GM25018, 2510003B16RIK, OLFR156, 4933430M04RIK, HAVCR2, RHOX5, CELA3A, LRRC74B, A530006G24RIK, 1700016P04RIK, GM1140, PCDHGA12, RETNLA, PCDH12, GM12505, MIR9-1, GM5538, PRSS45, RINL, GM26839, GM5886, WT1OS, 4930564C03RIK, 4930532103RIK, ANKRD23, PLATR4, GM30551, ZAR1L, GM11529, FTCD, GM20319, MIR3089, MIR691, 2610027K06RIK, C730036E19RIK, MRGPRA4, GM5108, NUDT12OS, GM15631, SMPD5, MIR24-2, GM11482, ROBO4, ITGB6, MIR26B, 1700065J11RIK, MIR3092, MUSK, PARD6A, MIR378D, 2010001A14RIK, 2310001H17RIK, 1700123020RIK, LIPO3, VMN2R58, MIR6968, OLFR95, 4930461G14RIK, GM27162, MIR7067, GPR141, MIR6403, RFPL4B, SULT2A8, GM12633, LOXL1, 1700066C05RIK, 1700121L16RIK, PCNX3, MIR3058, D230017M19RIK, GM44501, A930001C03RIK, OBOX7, GM6961, MIR6354, H2-M10.4, CLDN34C4, 1700061F12RIK, MIR3080, VMN2R24, CSMD3, TBX5, LBX2, 4930519F24RIK, 1110028F18RIK, CYPT14, MIR511, PTPRCAP, ADAD1, GM32014, IRGC1, FAM104A, GM1968, 4930480K23RIK, APOA5, OLFR470, MIR1982, SNORD12, SLC6A16, JMJD8, H2-K2, 1700030020RIK, 4930518P08RIK, TRIM58, PRSS21, 4930548G14RIK, 4930533N22RIK, GM28979, GM30539, MIR10A, OLFR197, 4930539C22RIK, FHITOS, VMN2R6, SLC13A1, ZFP648, GM10684, IFI27, OLFR49, GM5177, MIR295, 1700095J12RIK, GM15939, WNT1, ARHGAP150S, OLFR294, MIR7094-2, 1700027J07RIK, 1810021B22RIK, TMEM217, FAM170A, RNASE13, 1700101022RIK, MRPL43, ATG7, B020004C17RIK, PAX4, 4931417E11RIK, MIR1905, STPG3, 4930592C13RIK, VMN1R64, PKP3, 4930404N11RIK, FENDRR, 4930425L21RIK, IQCB1, ADORA3, UGT1A10, GM4792, GK2, GM4432, 9330178D15RIK, GM732, IL13RA2, VMN1R100, GM29678, VMN1R60, LNCPPARA, 4930546C10RIK, MIRG, MIR7671, A530053G22RIK, MIR1906-1, MIR6379, VMN2R75, CCER2, GM32856, 1700039M10RIK, MIR7032, GM17751, RHOX3E, SPEER1, 2310001K24RIK, GM13944, 4930529K09RIK, ARHGAP8, OLFR418, GM44504, ZFP408, GM12911, GM26682, BC048644, A530058N18RIK, MIR7085, MIR6348, SPACA7, AGBL4, MIR3079, MIR383, CLEC4F, MIR21B, GM36582, VMN2R13, MIR105, GM2115, LKAAEAR1, SRPR, FOXI2, HOXB6, RHBDD3, FAM181A, PCDHGB8, MIR8119, GM6559, C030018K13RIK, NIM1K, DEFB7, PRDX6B, 5830428M24RIK, HSDL1, ZFP319, VMN1R27, 4930474N09RIK, SNORD14C, A330008L17RIK, MIR449C, A930033H14RIK, SNORD88A, GM30400, GM11651, MIR8095, GM32921, MRGPRB8, LNCENC1, MISP, GM7072, MIR18, IK, USF3, GM36738, E030011005RIK, 4933416E03RIK, TUNAR, IQCF1, 2310008N11RIK, PBLD1, A730020M07RIK, OLFR1305, IL1RAPL2, SPDEF, MIR99AHG, ADAM28, GM10375, A630072M18RIK, APOL11A, TNNC2, 4930505G20RIK, FMOD, RBM12, GM20356, CFHR1, CYB561D2, SOSTDC1, GM15543, MIR181C, 4930428021RIK, TGM6, E030018B13RIK, GM12381, 1700001GO1RIK, 4930435F18RIK, P3H3, 4930542M03RIK, EVX1OS, 4930444M15RIK, OLFR725, 6030407003RIK, GM4988, LRRIQ4, KLRA17, PDILT, 4933421107RIK, 4930500F04RIK, MIR208B, MIR6376, GM21188, MIR686, CNR2, DLL3, GM7904, GM20257, SLC25A27, NAT8F4, 4930591E09RIK, SLC34A3, SLC9C1, 4930578N18RIK, KCNMB40S1, A330093E20RIK, IL411, LACTBL1, TAS2R123, 4930401012RIK, BACH2OS, 4930513D17RIK, AMHR2, GM25212, OLFR478, ZPLD1, ANKRD60, 4930486103RIK, CD36, LILRB4A, ZFP740, MIR3063, GM19935, METTL21C, GM41341, PDIA2, TMPRSS3, GM20110, GM8765, HOXB7, 4833422M21RIK, 4930527G23RIK, 4930425010RIK, LOC102634389, D030024E09RIK, AGTR2, 4930453L07RIK, CST10, TTC36, LOXL4, GM17745, OLFR31, MIR6953, OLFR982, B230209E15RIK, GM29443, GM15091, QRICH2, MIR6409, DEFB48, A230107NO1RIK, MIR7235, OLFR1057, GM5934, SERPINB6D, MIR21A, GM17753, GM29687, OLFR502, GM4951, 4933402D24RIK, EPS8L3, OLFR340, GM44502, MIR7076, GM6760, GBP11, HORMAD1, ANKRD27, MIR3100, GM16701, ZFP966, VIT, DCST1, 4930587E11RIK, VMN2R55, AANAT, SLFN9, TBXAS1, OLFR1306, LOXL3, MIR6896, FAM219AOS, OLFR952, 4930434J06RIK, GM36117, 2310043021RIK, CHRNA10, KIF19A, OLFR455, A930007119RIK, H2-M9, MIR145A, 1700015F17RIK, CARLR, TCF23, GM16364, GM28590, A830035019R1K, GM10549, 5430405H02RIK, VMN1R48, VMN1R234, VMN1R91, GM12794, GM16404, TPRG, 1700010K23RIK, SHBG, GM11729, GM15723, CRYAB, 2410004101RIK, GPR174, OLFR786, GM853, NRIP2, GM5795, CD209C, AMY2B, CHILE, 2810025M15RIK, PARP9, OLFR177, CELRR, GM5570, NLRP1B, GLT8D1, OLFR566, HSD3B5, PVT1, GM6614, 4930579D09RIK, CKM, 1110028F11RIK, E330016L19RIK, MIR6537, KHDC1B, MIR6927, MIR1191B, ZSCAN10, 3110039M20RIK, A330076C08RIK, GM20745, VMN1R89, LGI1, GM4832, MIR6344, KLRA2, GM20125, 1700108F19RIK, 1700100I10RIK, DKK4, OLFR279, GM5524, GM14405, MIR137, MIR6346, GM28626, COX7A1, VMN2R114, GM9871, TBC1D22BOS, 4932414N04RIK, MIR6899, GM38670, GM12339, HOXB5, GM10754, OLFR987, 4930555B12RIK, GM10665, RNU11, 1700001F09RIK, and 9330104G04R1K.

H3K27me3-high genes in mouse FTW-ESCs: MYH11, BANF2, LAYN, IER5L, CACNA1A, TRMO, OPRL1, SMIM23, CIITA, PTK6, LRCH1, SBSPON, IQSEC3, CRYAA, CD34, CCKAR, CYP1A1, CMYA5, CCR6, DPEP2, PTX4, PYGB, NRARP, DACT3, FN3K, SSPO, KHSRP, B3GNT3, NKAIN1, PHLDA2, TPBGL, TNNT2, TTC8, CHIC2, ZAN, KCNA6, SLC22A18, ETNPPL, SMAD6, SH2D7, KLHL26, CARD19, CTTN, GBF1, MCM5, KISS1, SUNS, CALB2, SLC4A1, MARK4, CPT2, ADGRL1, SPPL2C, KLK15, SLC34A1, CFAP100, SLITRK1, SPATA3, ZAR1, PSMB1, CACNB1, MESP2, NPBWR1, VPS37D, SDS, SAYSD1, MC3R, CD247, FBXO39, CST7, ADGRG3, SNPH, MAF, ANO1, BNIP1, BTC, TOX2, KRTDAP, CCDC185, TNNI1, FOXL1, NOL4L, CCND1, SUN2, ARID3B, DCAKD, NUP210, SNX20, CD19, PLPPR4, SSTR3, GRM7, KCNK3, SMC1B, CENPE, SYCP2, EPHB6, PEMT, KCNA3, KLHDC7B, PPT1, DCC, RAC2, TSN, KBTBD13, TMED9, SRC, SPARC, KDF1, ENTPD6, SLC2A10, TTLL1, AKAIN1, KCNA10, RIMS2, ADAMTSL4, FGF20, PLD4, CYTH2, EXOC3, SHE, ZFP37, BTNL2, CFAP74, UBE3C, CMTR2, PSMG3, SDE2, SMTNL1, CEMIP, TEX29, SLC29A2, PTGDR2, KCNIP4, PTPN20, MYOZ3, VOPP1, ZNRF4, BAMBI, DENND2D, ADGRE5, NMUR1, NMRK2, VSIR, COL9A3, GRXCR1, VWF, EXOSC3, MIOX, BOLL, DGCR2, KRT74, INPP5J, NPDC1, ANGPT4, RD3, DUSP22, ARC, FCMR, TMEM196, RAD54L2, FUT7, GRAMD4, MB, CHMP6, A1BG, RASAL1, SGCG, LYPD5, SEMA4A, SLC30A4, SFTPD, PDCD1, SVOP, EMP2, PLPPR1, RAPGEF1, CD48, ADAR, KLHL35, ASTL, RETN, CCDC70, TMPRSS9, TOLLIP, MLXIPL, HAS3, AKT3, FCRLB, PRKAB2, MED15, OSBPL5, SIX5, NXNL1, LRRC71, DYRK1B, CHGA, SLC46A1, ATF5, CHAC1, STAM2, DHRS3, NOD2, TRPV1, SRXN1, GLRA1, ACP7, DYNLRB2, ACBD3, FDXR, GRIN3A, PGLYRP2, IFNE, CCDC86, PLPPR3, C5AR2, KCNG4, CTSF, HSD17B12, GNG12, ADAMTS15, POU3F1, PSCA, MDH1B, C2CD2, NKX1-1, HAST, SULF2, TTYH1, FKBP6, IL18, SLC10A4, NRF1, LEP, MFSD4A, UCP1, TMEM141, SLC18A2, TBC1D16, MEIOC, TXNDC16, PCSK9, CDKN1C, TNFRSF14, FCHO1, PTGDS, RABAC1, ZNHIT3, KRT82, PROM2, PDZRN3, SBK2, PDIK1L, HSPA12A, ANKRD35, ADGRG1, ADAMTS10, TEX36, SLC2A4, LARS2, RBM15, NECTIN1, SLC8A2, ERICH4, NAGPA, VWCE, PLPPR5, CEP72, KCNIP1, NCAM2, C1QB, KDM8, GPR176, DRD1, PISD, TRIT1, PFN3, LPL, SORCS3, RNF169, TMEM92, HERPUD1, PDE6B, KCP, BRF1, MOB2, CSNK1D, MIB1, PDGFB, ERN2, HPD, SLC25A41, PDZD7, RTKN, B2M, ACR, NEK6, ZHX2, FADS6, CHURC1, KLF10, SLFN14, GABRA4, AGBL1, FAM162B, NUP93, PSMC4, ACBD6, COL4A4, ILVBL, POP4, GOLIM4, PDE1B, ATP6V1B2, TRH, ADGRG5, AKAP5, NFKBIE, PRODH2, KRT4, JAM3, GPR61, CACHD1, SBK3, PLPP4, ERAP1, IL12B, RARRES2, PRKAG3, CFAP58, SYT5, DENND1A, QSOX2, EPHX1, CLP1, ARHGEF10L, EIF4E1B, HSD17B2, MPP2, STUM, FAM166A, TPCN2, TM9SF4, DMRTA1, CLPTM1L, HEMGN, EXOSC6, ACER1, TMEM176B, INCA1, WAC, VSIG10L, ARL5C, CELF5, TNS1, IL15RA, TRIM10, DPEP1, EXOSC9, HERC1, ANKRD33, TBCD, UBE2T, CCDC183, MTNR1B, CD300LG, PGLYRP4, PRR5L, RAB40B, ALKBH5, STAP2, ARL2, IGLON5, DISP2, ERICH5, CCL24, CDH16, TMC7, JAK3, LMTK3, LIN28A, CDH5, NROB2, LTA, CCDC130, OTOS, LRRC27, RAB11FIP1, PSMB7, MAP2K3, HILPDA, LMOD1, CFAP77, SPATA32, FAM204A, CYP4X1, KIF12, GPR149, KCNA5, MST1, TTC4, LRP3, PRR16, NEU4, UPK2, CCDC174, PTH1R, MYT1, LY86, TROAP, NINL, TRPM5, ARMC1, SPNS3, CHRNB4, KDM2A, MEF2D, MEIS3, SHISA8, SIN3B, FOSB, XKR6, MYL10, USP31, PLPP7, ASPDH, TBX5, KLC2, AQP8, SERTAD4, TH, KCNF1, EPS8L2, OLFM4, CASC4, BAIAP2L2, ENDOV, UPK3A, MOGAT1, SLAMF8, TPM4, STRBP, RUFY4, FAM221B, CRB2, PRICKLE2, TMEM176A, ARTN, GPRC5A, CTBP1, WDR61, GPR45, DHCR7, LZTS1, SPTLC3, PEBP4, KCNQ1, LKAAEAR1, NXT1, ALDH1L1, TMEM9, GIMAP8, TTC6, TACC2, PACS2, ELMO2, GLDN, SEMA4B, FBLIM1, COL20A1, CNR2, MCOLN2, SLC22A12, INSR, POLG, PELI3, PPP1R1A, TNFRSF13B, AQP4, SSC5D, CYB5R4, CDKN1B, NDE1, SLC16A8, SLC46A2, DAO, LPAR5, PCK1, YES1, FXYD6, SULT2B1, NLRC5, SAMD11, TBATA, TNFRSF18, PHF12, NUB1, FREM3, IL12RB1, HAGH, NWD1, LYPD4, CNDP1, PCMTD2, KCNG2, TMEM200C, RNH1, H13, MIR6980, CRLF2, 4930524008RIK, GM4981, GMFB, TTL, COX19, TLN2, TFCP2, AY702102, ADAMTS6, EEF2KMT, TMEM15000S, MIR124A-1HG, DDI2, MIR5120, SUMO1, GM37013, GM7550, DHRS1, ZFP638, MIR6976, TVP23A, SLC26A7, FHL2, ZCCHC7, CTNNA3, 6030466F02RIK, IL13, GM20765, CLPB, 3110045C21RIK, 2610203C22RIK, IRAK3, CYCl, 4930471M09RIK, RBP3, APBB1, ZFP422, GM21671, HOTTIP, SCG3, CHCHD3, 4930505K14RIK, IP011, ARID1A, MC2R, F830045P16RIK, ZFP12, MIR6991, ZXDC, RIPK3, RHOG, MIR3107, SMYDS, PLPP3, CHCHD6, TXNDC9, TBC1D12, MIR193A, TRIM68, SCAMP2, NDUFS4, MIR874, MRPL35, PTPRN, IPP, COL4A3, GPR84, TRP53BP1, E330017L17RIK, 9530077C05RIK, ZFP354B, MKRN2, TIMD4, CYP4B1, CD209F, SUCLG1, JUNB, H2-DMB1, UBE2J2, 4933438B17RIK, ALG10B, ZFP30, GM32511, GM13283, TEX37, OLFR12, CSTAD, NSMCE4A, 1700030C10RIK, PLA2G3, 2510039018RIK, ANGELL GM17767, CRH, PRMT6, GM30173, OLFR1419, FOXJ2, NPCD, AI661453, FIBIN, DCPP3, RTCA, SNRPB2, CAMSAP2, TAC1, MOS, OSM, TEKT4, CLK3, BC048671, 4930563H07RIK, MIR615, GM6034, MPZ, RANBP2, TNPO3, OXGR1, FXYD2, 4930445N18RIK, ZBTB46, EYA3, MEDAG, POM121, PPT2, GM38426, TPTE, ANKRD42, PTPN2, DLX4OS, RNASET2B, SIAE, YBX3, NES, GM21119, UBL4B, GM16596, GM19689, CBLN3, CES4A, GM1715, PCDHB6, 5031434011RIK, ERCT, 1700095A21RIK, CDK5RAP3, ZFP322A, ACER3, ERICH6, GULP1, AFG3L1, 4930440I19RIK, 4930503B20RIK, KRT16, SPCS2, CWH43, GM7102, SPP1, LCN4, CXXCS, PCDHGB4, UGP2, 5031425F14RIK, SPINK4, TRIM30D, APLP2, ZZEF1, NHLRC4, GM32455, CD74, H2-Q9, PPM1G, PCDHGA6, HOXAAS2, 9330182014RIK, YPEL1, STAMBPL1, 2900040C04RIK, CTLA4, PRDM9, TSC1, TTC39A, DERA, PCDHGA3, ZFP59, 4930413E15RIK, 4933412006RIK, DAPL1, SETD2, G2E3, 2700054A10RIK, RAD18, H2-D1, CTU1, 4930520004RIK, PCDHB5, NUS1, CCL22, TMUB1, GM10687, CREBL2, GM10710, TAPT1, RPGRIP1, BHMT, ZFP329, IGFBP2, POLD4, TLDC2, ZFP961, AGRP, IN080, PCDHA5, AKP3, KRT14, PINX1, MIR5127, MIR6769B, SNTG1, CD300A, ALMS1, NUDT15, FAHD2A, CMTM3, PRR23A2, PSMB11, CLCN1, RESP18, PAIP2B, PANTR1, SIGLEC15, 1700022E09RIK, AU041133, ABHD16B, YLPM1, ERP27, MIR5104, MIR152, INPP5F, MATN2, MIR7241, FCRL6, TSPYL4, GM23450, SLC7A6, 4930402H24RIK, USP8, BTNL10, GSTT3, AHSG, CYP4A12A, GM14023, ADGRL4, GM16582, HILS1, TRPC2, 4930556N09RIK, MICALL1, TMED3, E230013L22RIK, KLK4, LIN7C, FOXN1, TRAK1, MIA2, ZSCAN18, SEC61A1, TTC14, MIR3108, GM1987, RFTN2, GM536, RNF213, TRAF7, PRDM4, GM7788, PARL, MIR6944, GM2109, BPIFC, SLC2A9, 6530402F18RIK, PCDHB19, A430033K04RIK, H2-M3, 9230112D13RIK, CCDC175, EPB42, OBOXS, SAXO2, RBAKDN, SMC2OS, KLRA1, 1700044K03RIK, DPP3, TEX30, GM15421, PCDHGA9, E130018015RIK, SLURP2, PUM3, 1700095J03RIK, CCDC102A, SLC30A8, 4930558C23RIK, CD209B, ACOT12, 6330415B21RIK, PCDHA9, HPGD, CHMP1B, LOC100861615, 3110082I17RIK, PPP6C, ATXN7L1OS2, B230208H11RIK, KIDINS220, ACTL9, OLFR521, GM33727, 1810026B05RIK, ZFP105, ACKR1, TMEM210, 2310015A10RIK, MPP4, SMIM17, A130077B15RIK, 9530080011RIK, GM4793, MIR6420, FFAR2, GM3942, TBC1D19, 1700063D05R1K, 8430430B14RIK, 4930579F01RIK, 1700040D17RIK, NRBP1, CNN3, 1700009C05RIK, GM26684, TFPI, ZFP787, AI839979, TLR9, MRVI1, EXPHS, H2-T3, GM4956, SSH2, 5430421F17RIK, DUSP13, TLCD2, GM1943, CEACAM-PS1, 4833412C05RIK, 4930431P03RIK, GM10471, 4930483008RIK, AADAT, MIR6416, NR1D1, FYB, MIR671, MRPL9, IL17RC, 4933433H22RIK, ZFP133-PS, GM12669, EID1, GM15908, PAPPA2, A330048009RIK, A230051N06RIK, HEPACAM2, GRN, BLOC1S6OS, ANKRD13C, GM26633, DDX50, PBX4, GM10377, C030005K06RIK, MT4, MAP7D1, 4921509C19RIK, SH2B2, ARHGAP39, ZFP51, GM15569, CD209G, MIR3968, 4930404H11RIK, MIR5046, PCDHB10, FBXO3, ZFP26, 4933430N04RIK, GM17733, MIR692-2, STRIP1, BFSP2, SHMT1, DCT, VEZFl, GM10532, LNCPINT, GM21190, C4B, 1700109G15RIK, ASB18, MAGEF1, GM15501, MIR1963, GM38404, RNF145, COX6A1, STX18, ARMC6, GKS, MIR7011, 1700034G24RIK, NAB2, GSDMA2, MSANTD2, STKLD1, FNTB, GM5577, ZFP109, FAM45A, KLHDC1, KLHL31, MORNS, SAP130, ZFP950, ZFP541, HALR1, 2900041M22RIK, GAL3ST3, EIF2AK4, PCYOX1, CLCN7, 2210404E10RIK, 4930483J18RIK, MIR3069, AP1G2, MRGPRF, CES1A, GM10466, TLK2, ZFP768, GM17634, TERB1, MIR5118, BCL2A1C, SPX, RAB3D, MIR2139, CCDC42, MIR6395, RHEB, OFCC1, RIDA, GM13547, SOD3, TWF1, NSUN5, DGUOK, MPO, PREX2, TEX261, DUPD1, GM20219, PCDHB2, ZFP235, APOL7A, VMN2R57, SERPINC1, COG1, SDHAF3, 4930426L09RIK, GM5523, ZFP688, TAS1R2, MIR8120, A630023P12RIK, GM20767, MIR7213, GGA2, 6430628N08RIK, HSD3B2, MAT2A, ZFP964, GM9833, 4930432J09RIK, GPR85, ACTL10, CLEC4A2, GM11978, UNC119, RAB2A, 2310002F09RIK, PLA2G4D, LYPD2, 9030624G23RIK, CRCT1, MIR6973B, FBXL19, ZFP746, CXCL3, ALDH3A2, PKLR, SPR, KLHL21, ITGAL, AP3S1, ZFP512, GM14486, SENP7, ASMT, NARS2, FSTL1, TM6SF2, UBOXS, TOM1L2, ZFP947, ZFP212, TMCC1, BTBD7, ULK2, GM6639, 4921504A21RIK, OLFR463, PRR30, PCSK2OS2, 4933400L20RIK, ZFP110, MIRT1, FSD2, ZFP612, MFSD4B5, CPNE6, MIR7047, PDXK, IRS2, CCL21B, LMAN2L, FASTKD1, NCK2, SYCE1L, HUNK, NOMO1, ANKAR, TMEM181C-PS, CYP4F18, MTMR3, GM16677, FGG, SP7, HPF1, ABCA6, SREK1, 4930478P22RIK, FCER2A, PCDHB12, PCDHGC4, PLEK, CYP4F40, PRND, CLEC1A, CACUL1, ZFP 131, SKINT10, NAP1L5, MPST, LRIF1, HRC, MFSD4B1, ZFP239, ZFP511, LRRC3C, CELF6, SNX31, TBKBP1, GM5111, RSPH1, LSM8, PCDHGA7, OLFR545, DNAJCSB, SMCO3, PCDHB22, UBE2V2, TIGDS, MIR142, PCDHGA5, 4930557F10RIK, PCDHGB7, PSMD5, IFI47, CES2B, 5033406009RIK, GPX6, MIR205, GPR19, MIR7214, 4930523C07RIK, TOMM20, PABPN1L, GM14057, 1700020N18RIK, MIR7000, CFAP206, 9130204L05RIK, ECI3, PCDHGB5, MSI1, SPAG17, TCL1B2, GIMAP1, B4GALT1, PPP1R2-PS3, GCFC2, PRSS2, TLR12, GM30731, 4631405J19RIK, GM3002, PCX, GM16617, NKAPL, 4930558K02RIK, RBM34, DNAJA4, DNAJB6, MIR7K, A430005L14RIK, 4930511A02RIK, IGFN1, ADCK2, COPZ2, ZFP583, GM9731, SPDYE4B, PLATR22, SPRN, SMIM13, PTF1AOS, OTUD4, SEC61G, NKIRAS2, WDR17, 4933422A05RIK, OGDH, CD1D2, 1700006H21RIK, MIR6906, GM10639, GM15972, DTNB, PCDHGB2, CFI, CTSL, PSMD14, RAD21L, MED4, 1700073E17RIK, SSBP1, 9330198105RIK, CALU, OPTN, GM6712, GM9530, PCGF6, ZFP707, GM38438, 4930506C21RIK, C530044C16RIK, OLFR750, RDH9, PXMP4,0 MIR6239, PCDHB21, TGM4, LCE1M, HERC3, PEX11A, KLHL22, 4933432109RIK, OLFR449, 5430431A17RIK, UBXN10, MIR592, 4930550C17RIK, TOMM70A, TAC2, GZME, ZKSCAN4, OAZ3, ERGIC2, PHGR1, 9530052E02RIK, TMCO5, FCGR1, SLC36A1, BAG3, PALLD, 1700125H20RIK, GM29684, RBM6, ZFP572, MIR7021, ZFP266, APOBEC4, PGAM1, 4930519G04RIK, RABL2, 3300002108RIK, ACSS2, RPS27L, GSTT4, ARHGEF18, PLCXD2, 2610206C17RIK, ITGAE, GNGT2, 1110036E04RIK, MIR6362 DR1, PANTR2, HPDL, EAR10, 1700016C15RIK, CALHM1, ACBDS, CRIP3, FST, 1110038F14RIK, FUT4-PS1, 4921536K21RIK, CYB5D2, GM15997, ACAD9, CLDN7, HSDL2, ACVR2A, HTT, IRS3, FUT2, TAOK1, 1700066J03RIK, PON2, TLR2, 4930524B15RIK, SEC11A, VTN, HSFS, 9430078K24RIK, OLFR1368, MIR6358, TUBG2, 1810044D09RIK, PCDHGA2, PBP2, PTPRV, BTG2, MLLT10, GJA10, AW011738, NEUROD6, MIR3083, GM13446, H2-Q6, GM5105, H19, MIR6378, GALNTL6, RPL17, GM6083, 4930522017RIK, PCDHB17, FADS2, ARHGAP27OS3, GM5424, CHRNE, PCDHB18, DTX4, 4921529L05RIK, CAR14, ACTR3B, LMO3, CCIN, GM4559, 9330175E14RIK, A230009B12RIK, ZRANB1, TRAF6, CRY2, HOXB5OS, 6030471H07RIK, NAALADL2, ZDHHC13, FBXL12OS, SLC15A5, GM3363, 1700020M21RIK, TGM5, MYCBP, IL17RE, UPK3BL, MIR7216, 2610316D01RIK, ODF3B, FEZ1, HMGCLL1, CYP4F14, DPPA3, ACTL11, GM36246, D030068K23RIK, ARIH1, GPS, 5430434115RIK, ISOC2B, C430042M11RIK, HFE, DMPK, 1700064M15RIK, 1700001G17RIK, C2CD4D, AW549542, 1700016K19RIK, GM16675, WDR1, 3300005D01RIK, ACACA, MAPKAPK5, RDX, PRICKLE4, F2R, GM4285, SIRPA, DDX18, PLPP2, PCDHA10, NCKAP1, PCDHGA11, PIGG, GM8633, TMEM266, ATP9B, ELOVL5, RACK1, MTMR9, MIR6974, H3F3A, ZFP931, GM10584, GM13238, MAPK12, RSPH9, JHY, RCN2, ZMAT3, ALKBH3OS1, PCDHGC3, LNCBATE1, GM26689, HMGN1, GLRX2, KRTAP5-4, ALG8, GM5868, CPE, BC003965, MRLN, KCNIP3, OTUD6B, GAL3ST4, GM17619, BGLAP, PCDHB20, ORC6, FASL, CCDC166, S100A5, TNFRSF1A, MTPN, GPM6A, ZFAND6, 3300002P13RIK, GM9999, A430078G23RIK, SPATS2L, DHX8, CHILL H2-T-PS, 4933406G16RIK, TRPM1, POLN, MIR7225, MRGPRD, 2200002J24RIK, MIR6367, FGFBP3, CCDC92B, 1700029F12RIK, BC147527, TTC39AOS1, 1700094M24RIK, ITPRIPL1, LRRC61, MIR8090, FLRT2, KNSTRN, ERCC6L2, GM26688, ANKRD36, MALL, TRIM42, GZMK, ACSBG1, NAGLU, ZFP791, KRT2, MRGPRE, ATPSK, GM38414, KBTBD2, CLSPN, CAPZA3, ACTL6A, SMU1, IQCK, GM15825, PCDHGA10, 4930447F24RIK, C1QTNF4, ACTG2, SLC25A10, D3ERTD751E, TOMM7, SLC22A21, GM29683, MFSD4B4, PPA1, MAST2, PELI1, A730006G06RIK, GSDMC4, 1700001C02RIK, SPINT5, ZFP467, MIR7221, MIR6923, 4931408C20RIK, PCDHGB6, RNF32, VWA3A, EEA1, PCDHB16, LAMTOR4, ZFP62, COL28A1, MIR8110, LRRC25, COQ6, BORCS6, HBA-X, ESRRA, RIOK2, TBL2, TMEM235, ZFAND5, PCDHGA4, NR1D2, C030029H02RIK, U2AF1, VWC2L, ZFP759, GM5142, PPIG, MIR6240, HERC2, LCE1D, MIR1953, 9430091E24RIK, LRRIQ1, GPX3, RCE1, CCR9, HBQ1A, PCDHB7, YY1, MIR146, GTF2F2, LOXL2, TMEM120A, GM5294, TEKT5, ZKSCAN1, ZFP433, LOC100862268, SPOPL, CPNE4, MIR466F-4, CCDC91, KCNQ1OT1, ELOF1, GM16630, 4933406C10RIK, 1700017L05RIK, PCDHB4, MIR149, 2310011J03RIK, HCRTR2, DCLK2, MGAT4C, MIR145B, STK25, 4932438A13RIK, L3MBTL4, MMP2, RAD52, B3GNT6, MIR7215, MED31, ZFP760, GZMA, 1700113B09RIK, KIF3B, SSTR4, SLC35B1, SYNB, AP1M2, HCAR1, SLC24A1, KRT87, DNAL4, ZFP704, ZFP128, DNAJC13, MIR568, TFF3, DMRT1I, RABGGTA, GM11468, BBS5, SPEER9-PS1, SYCP1, GM5122, 1700113H08RIK, SHPK, RLN3, ATF7, ADAM29, OLFR215, PCDHB9, THUMPD3, EDRF1, LY6G6C, PPP1R32, AMH, GM38509, D6ERTD527E, FAM90A1B, MIR3090, D5ERTD579E, BTNL9, GM23363, MSH5, PNMT, TSGA13, 1700049E22RIK, BC002163, TMEM74, 4933402C06RIK, PUM2, GM7457, 4930519K11RIK, PCDHB13, 4732414G09RIK, NDN, GM6588, FGF6, MRPL48, MRO, SLC13A2OS, TSNAX, GM11681, MIR143, CMTM1, NCEH1, ABHD8, DUSP2, SLFN50S, 4930586NO3RIK, DNAJC10, ZFP286, TRIML1, ACYP2, CDNF, ACOT5, GM20268, GRIP1OS2, MIR3085, LAP3, SHF, BTG3, CNGB1, FBXW10, ERDR1, 4921504E06RIK, NHEJ1, MIR5103, SMIM14, ATP5G2, ADAMTS4, SPTLC1, PRR29, ITPR1, CD3G, MIR219A-2, SLC35F6, OIT1, UBE2M, FGF15, TTC30A2, MIR9-3HG, MCOLN1, GPX7, PPIE, TMA16, C030013C21RIK, WFDC3, ATP6V0E, 4930554H23RIK, MIR6925, PALMD, 1700012B09RIK, AU021092, 9430014N10RIK, A830029E22RIK, EXO1, ARHGAP15, GCM2, LHPP, RBM46, CLDN12, TEX12, SLC25A24, SIMC1, OLFR65, 4930565D16RIK, CHID1, 4933438K21RIK, CFAP20, AGAP3, MIR124-2HG, SMPD2, OLFR63, GM32141, PCP2, SNRNP25, GM10768, CPAS, PRSS29, DPH1, TTC1, TFF2, TRIM16, 4930579K19RIK, 1700086006RIK, CSTL1, ADAMTS13, NR1I3, CCDC182, DECR1, CYB5A, GM19434, CCDC184, ANKLET, 1700061117RIK, POLR2B, MIR135A-1, DYRK4, 8430437L04RIK, RSPH6A, CLDND2, 1810024B03RIK, ACOT1, ZGLP1, LYG2, ZFP2, NUCB2, IQCF5, FFAR3, GM7271, HK2, TAF11, RTTN, GM14124, HDAC10, MIR6921, 9330179D12RIK, EMSY, GM6644, RAB23, 4930515G16RIK, PSMB4, KLF13, HEXDC, FAM183B, EZR, 4933407E24RIK, DNASE1L3, PCDHAl2, CCER1, FYTTD1, SLFN8, LPAR3, ERI3, PDSS1, GM3336, A730018C14RIK, GM15326, 8030451007R1K, 6530403H02RIK, MIR5099, TUG1, PDXK-PS, ZFP747, TM2D1, PKNOX2, 5033428122RIK, CD1D1, SLC4A5, KLK11, BOLA1, KDELR2, TRP53I11, RTCB, CBR2, CCDC121, HIST4H4, MIR7B, A730013G03RIK, GM22109, FAM71F2, IDUA, GM29811, SCG2, GM15441, F830208F22RIK, PAUPAR, A930031H19RIK, 4632415L05RIK, SUMF1, RCBTB1, KTN1, GM13003, EQTN, GM3317, FBXL8, ARL8B, GM14424, GM31747, MIR10B, PCK2, PRDX1, SACS, S100A3, BBS4, ACTR3, CR2, GM3264, AI504432, ESYT2, PAK1, CES2A, ISM2, OLFR332, C87198, RPL5, EXOC5, 9630028B13RIK, SELENBP2, AVL9, DIO1, NTPCR, 4930524C18RIK, CD200, D830013020RIK, COPS4, FAM186A, LOC105245869, IRGM1, AA545190, TMEM115, MIR804, MIR155HG, PRSS34, F630040K05RIK, SH2D2A, ITGA5, IP6K3, A230050P20RIK, ZFP366, GM4425, EPHX3, CD300C2, 4933427D14RIK, MED6, HCLS1, SERPINA3F, POC5, 1700030M09RIK, 1700012D14RIK, NEPRO, 4930539M17RIK, AHCYL1, 4933402J07RIK, GM13031, AKAP10, H2-Q7, 4921513103RIK, DHRS7B, IGF2BP2, GM13848, NVL, TREH, PCDHA3, OLA1, UBB, DARS, TM4SF4, AMOTL2, MICU3, MIR6405, RORC, 1700037H04RIK, 4930487D11RIK, 4930405L22RIK, 1700030F04RIK, UEVLD, GM11346, LZTFL1, SLC25A13, AU023762, ZFP637, DBR1, SLC25A31, ILDR1, GM25018, 6820408C15RIK, DYNLL2, OLFR156, RHOX5, 4933431E20RIK, GPRIN3, DYNLT1B, CELA3A, VMN2R9, LRRC74B, A430090L17RIK, PCDHGA12, AI987944, TRP53RKA, GPR89, GSDMA3, MIR598, GM26839, PRSS22, GSTM1, FAR2, RIMKLB, PLATR4, ZFP101, GM11529, CLASP1, MIR1903, COQ10B, CRYGB, CLEC3B, ZFP939, 5830454E08RIK, ZFP41, GM15631, 1700060C20RIK, PKMYT1, ITGB6, ZSCAN12, C030037D09RIK, ACY3, CARSA, UFM1, PCDHB11, MIR378D, 1700123020RIK, DKK3, ZFP445, 4930461G14RIK, EPDR1, NBAS, MIR7219, RIIAD1, A530010L16RIK, PCDHA11, 1110059G10RIK, BPIFB1, GKAP1, HSPA12B, PCF11, RMNDSA, PACRGL, H2-M10.4, MIR3080, COG2, PSME2B, NOP58, JPX, ADAD1, METTL7A2, IRGC1, VPS45, 4930480K23RIK, APOA5, SLC22A5, GABRB1, MIR208A, MIR1982, SNORD12, 2610005L07RIK, SLC6A16, S100A6, S100A14, TRIM58, 4930451111RIK, 4930548G14RIK, GM28979, HMOX2, CD276, YWHAE, GM14317, SLC13A1, ZFP648, POGK, PDE6G, OLFR49, VPS13B, GAP43, 4921531C22RIK, 1700027J07RIK, MIR1895, 1810021B22RIK, ZFP386, GM5796, AP1G1, ZFP879, B020004C17RIK, TMEM170, KRT78, 4931417E11RIK, 4933430117RIK, HBA-A1, 4930592C13RIK, MS4A15, OLFR726, POGZ, SELENOF, MIR8092, 4930425L21RIK, SLC36A2, LLGL1, STATE, MIR670, SCGB3A1, SH2D4A, GM29678, TRAM2, LIPT2, IGDCC3, LNCPPARA, NDEL1, 4930546C10RIK, CD4, GM15050, GLCCI1, GM6313, WBSCR25, CCER2, COG6, MIR7032, SEMA3B, 1700072005RIK, 2310001K24RIK, GM13944, NR2C2, UBE2H, GM26682, MIR6348, SPACA7, TJAP1, GFY, MIR3079, KYAT3, MROH7, AQP7, CUEDC1, FAM13B, ATG16L1, TRAF1, MIR21B, GM36582, ZC3H8, RNF19A, PPIH, DIS3L2, FOXI2, RNF187, PTCHD3, PCDHGB8, FKBP9, GM6559, BDKRB1, P2RX1, DEFB7, BLOC1S2, INIP, 4930474N09RIK, ARPIN, ADAM6A, A330008L17RIK, GM11651, MIR8095, GM32921, UBE2W, TNFRSF23, AU040320, ITPRIPL2, HP, GGT7, TUNAR, IQCF1, GIPC1, CCDC106, 2310008N11RIK, PBLD1, MIR690, A730020M07RIK, H2-Q5, 5930430L01RIK, CCDC85A, MFSD1, FBXL20, CYSRT1, PTS, 4930505G20RIK, FMOD, CLASP2, MXD1, CYP17A1, RNF24, TDRKH, SOSTDC1, GM15543, E030018B13RIK, TSPAN3, 4930435F18RIK, 4930542M03RIK, EVX10S, DNMT3B, CYP4F41-PS, TRIML2, ZFA-PS, CAPN3, SYNPO2L, SLC4A10, DUXBL1, NGDN, MANSC1, MIR208B, GM21188, ZFP442, MFSD7A, CEACAM15, EMC7, NOVA2, KCNMB4OS1, GRIA4, FBXO46, HSPA1B, ACAP3, 2900097C17RIK, ANKRD60, MIR7086, UCKL1OS, GM19935, METTL21C, DDX27, TNFRSF10B, KLK9, TRIM17, TRPM7, 4930425010RIK, CCDC142, TRHR2, 4930453L07RIK, ABI1, B3GALT6, 1700017G19RIK, TCERG1, SLC25A2, GPATCH4, GM5779, MED9OS, TPSAB1, MIR7235, RPN1, TMC5, 4930553P18RIK, GM17753, GABRA2, DYNC1I2, EPS8L3, PCDHA1, SPATA31D1D, C1RA, NPM3-PS1, GTDC1, COL11A1, VIT, CHKB, TBXAS1, 6820431F20RIK, FAM219AOS, 4930434J06RIK, 2310043021RIK, RLF, A930007I19RIK, CARLR, SUOX, COA3, FIGNL2, PHLPP1, SHISA4, LTBR, 2610507I01RIK, RHBDD1, GM10636, SHBG, 2410004I01RIK, ESCO1, PMIS2, GM5087, AGO1, LRRC40, MRPL46, CD209C, PTPRJ, GYG, CELRR, CYP2D10, 4933417013RIK, PAPSS1, 4930579D09RIK, SNORD83B, SIGLECG, WDR7, MIR6537, KHDC1B, PPIL6, MIR1191B, PCNX2, FKBPS, PM20D1, 9130019P16RIK, NAF1, TICAM2, ELMOD2, ZFP454, GM4832, KLRA2, EVA1B, OLFR279, PCDHB3, CXCL2, MIR137, MIR6346, THRSP, UTS2, PTPMT1, 1810013L24RIK, RAB6A, 1700110I01RIK, 4932414N04RIK, MIR6899, TAF7, LIX1L, GM12339, MIR7066, GM10575, DYNLT1F, and LTA4H

It was postulated that formative pluripotency is associated with low mitochondrial respiration (Smith, Development, 144, 365-373, 2017). Studies described herein found that most mitochondrial complex COX genes and enzymes of the tricarboxylic acid cycle were downregulated, and enzymes involved in glycolysis were upregulated in FTW-ESCs when compared to ESCs, suggesting FTW-ESCs depend more on glycolysis and less on mitochondrial respiration (FIG. 10C). Another predicted feature of formative pluripotency is partial epithelialization (Smith, Development, 144, 365-373, 2017). The epithelialization status of ESCs, FTW-ESCs and EpiSCs was examined by growing them in self-renewing conditions in 3D Matrigel, and monitored rosette and lumen formation (Shahbazi et al., Nature, 122, 881, 2017). Results showed that FTW-ESCs could efficiently form rosette-like structures with limited capacity to form lumens (FIGS. 2F and 2G).

Albeit similar at the transcriptome level, FTW-ESCs and EpiLCs did exhibit distinct features. Most naïve pluripotency related genes were expressed at higher levels in FTW-ESCs than EpiLCs (FIG. 10D). On the other hand, a subset of formative pluripotency genes (e.g., Fgf5, Otx2, Pou3f1, Hes6, and Pim2) showed lower expression levels in FTW-ESCs than EpiLCs (FIG. 10D). H3K4me3 and/or H3K27me3 levels were consistent with differential gene expression patterns (FIG. 10E). Western blots using antibodies against several pluripotency related genes were also performed. OCT4 protein levels were comparable between FTW-ESCs and EpiLCs. However, in contrast to EpiLCs, NANOG was expressed at a high level and OTX2 was barely detectable in FTW-ESCs (FIG. 10F).

Taken together, our results demonstrate that mouse FTW-ESCs bear several molecular and cellular features characteristic of formative pluripotency.

### Example 4. Mouse FTW-ESCs Exhibit Chimera and PGC Dual-Competence

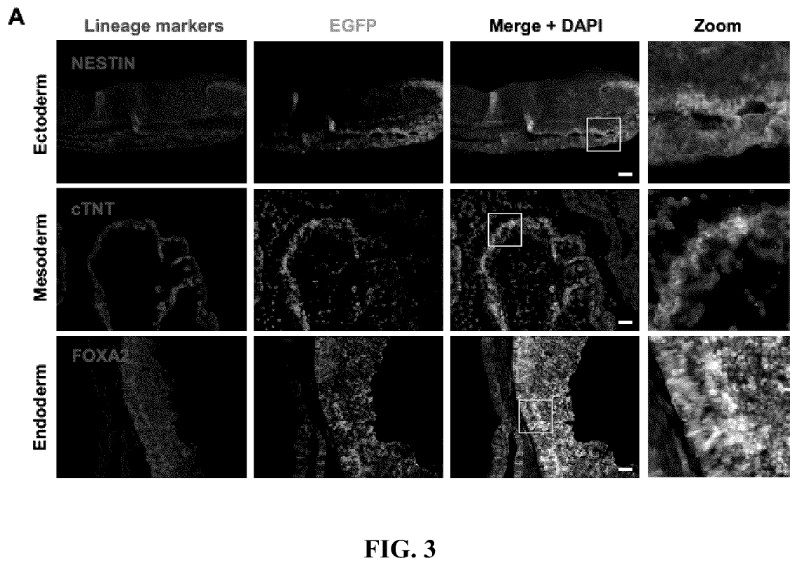

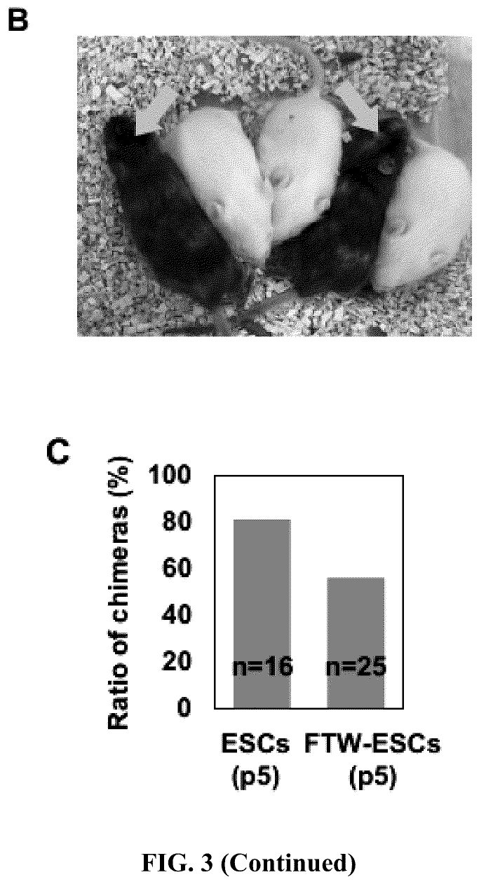

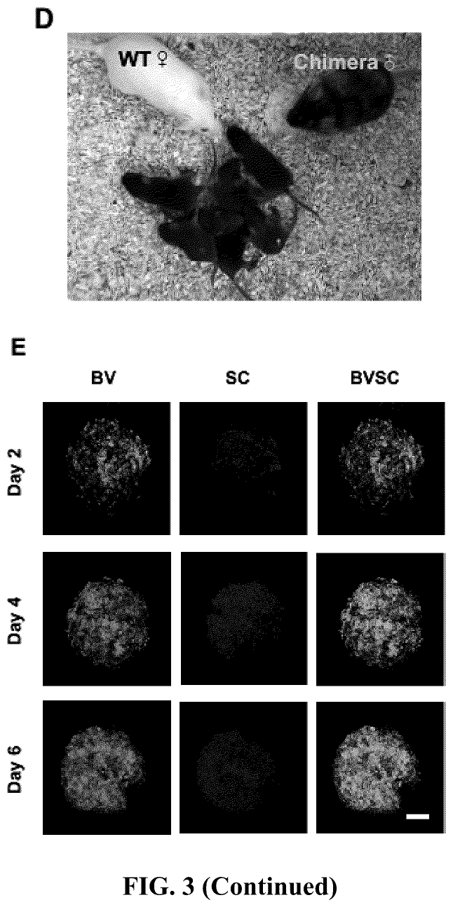

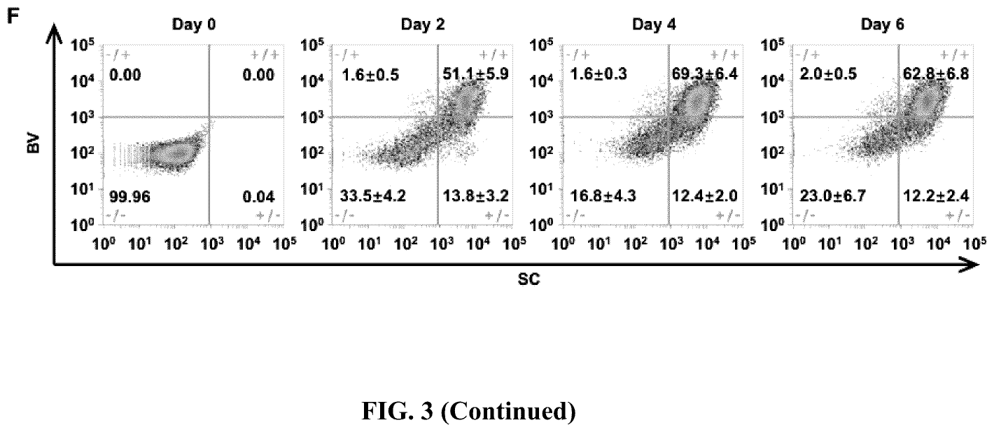

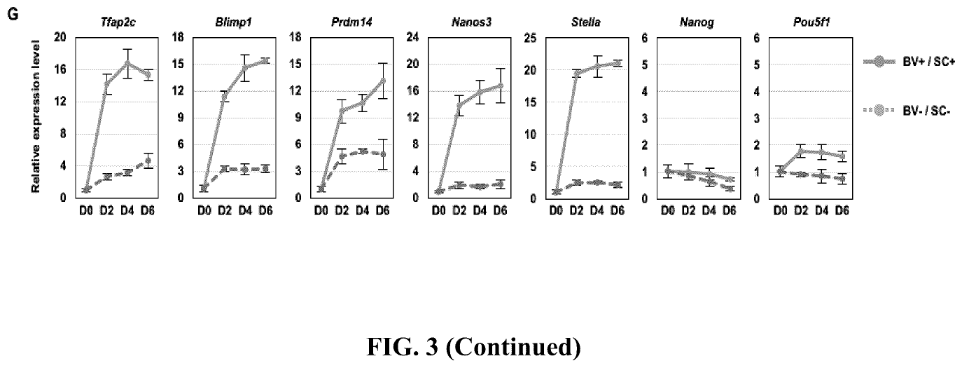

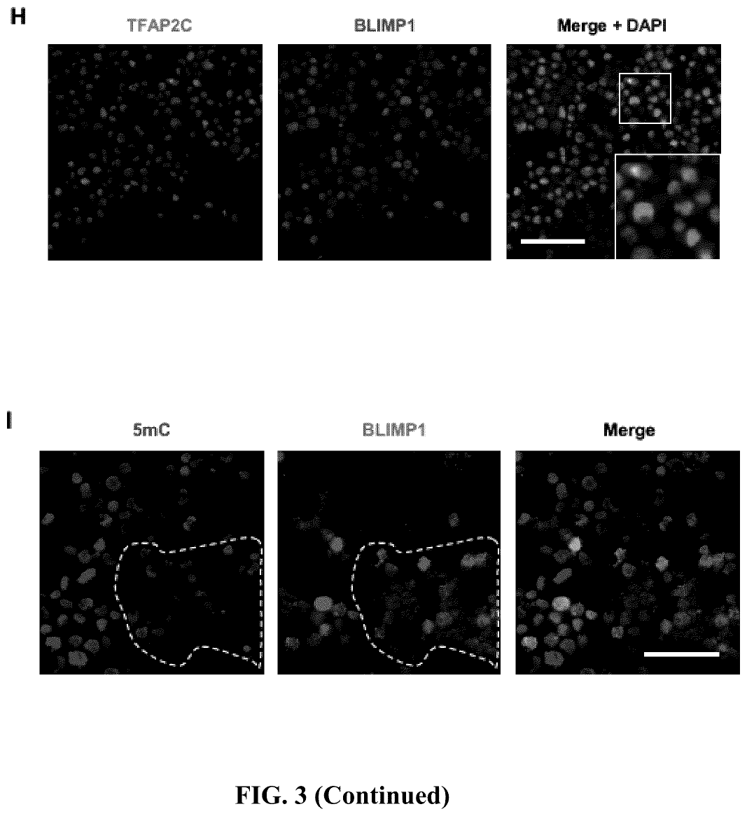

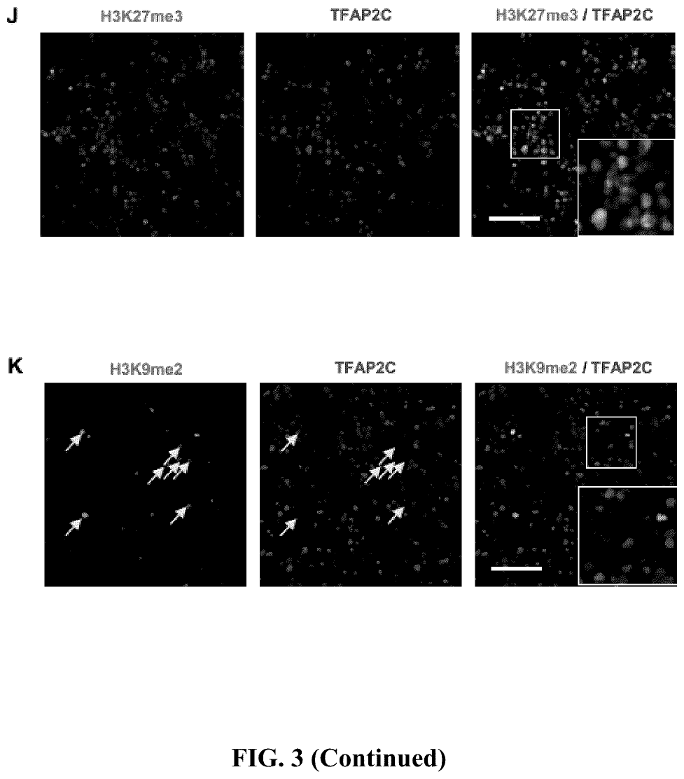

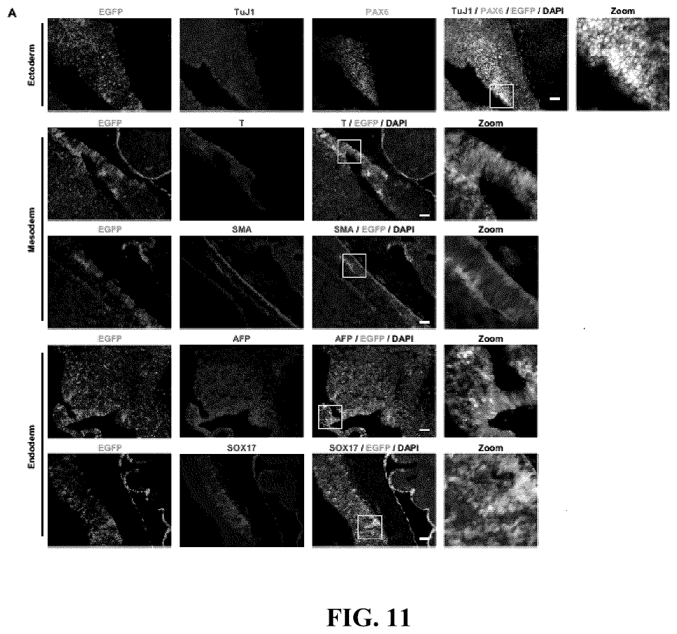

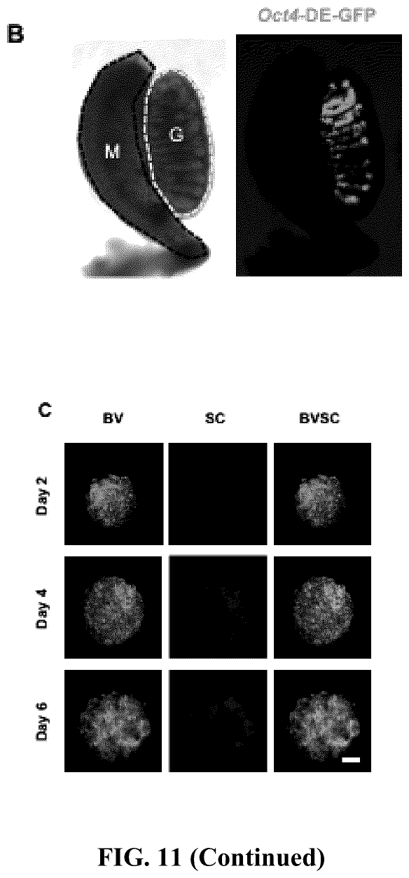

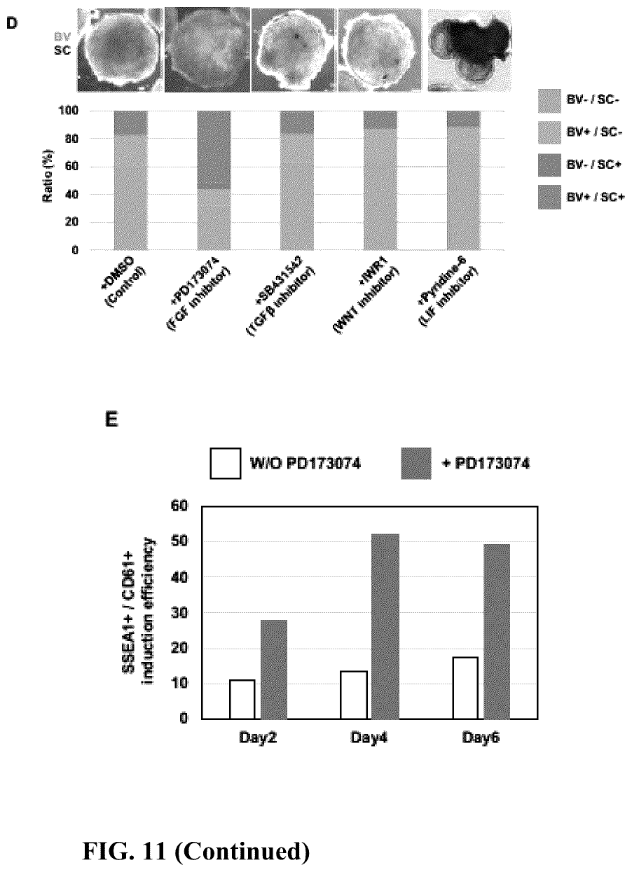

To test whether FTW-ESCs are chimera competent, EGFP-labeled FTW-ESCs were injected into E3.5 blastocysts followed by embryo transfer to surrogate females. FTW-ESCs extensively contributed to different fetal tissues and expressed lineage specific markers from all three germ layers (FIG. 3A and FIG. 11A and Tables 2A-2B). Adult coat-color chimeras were also generated by injecting C57BL/6 FTW-ESCs into either inbred albino B6 or outbred ICR blastocysts (FIG. 3B and Tables 2A-2B). The chimera formation efficiency of FTW-ESCs was compared with naïve ESCs by injecting the same number of PSCs (12 cells) derived from the same genetic backgrounds (C57BL/6), same sex (male), and same passage (P5) to E3.5 albino B6 blastocysts. FTW-ESCs were less efficient in generating coat-color chimeras than ESCs (14/25 or 56% for FTW-ESCs vs. 13/16 or 81.25% for ESCs) (FIG. 3C and Tables 2A-2B). To determine whether FTW-ESCs can contribute to the germline, contribution of Oct4-DE-EGFP FTW-ESCs to fetal germ cells in E13.5 gonads was examined. It was found that 6 out of 10 gonads contained EGFP+ signals (FIG. 11B). In addition, C57BL/6 FTW-ESCs derived chimeras were crossbred with WT albino B6 mice, and efficient germline transmission was observed (FIG. 3D and Tables 2A-2B). Taken together, these results demonstrate that FTW-ESCs can contribute to chimeras capable of germline transmission.

TABLE 2A
Summary of chimera results with mouse FTW-ESCs-fetal chimera results

| | | | Total | EGFP+ | |
|---|---|---|---|---|---|
| | | Dissection | Embryos | Chimeras | Chimeras |
| Cell line | Host strain | stage | # | # | efficiency % |
| B6 FTW-mESCs (EGFP) | B6-Tyr/B6 | E10.5 | 31 | 17 | 54.8 (17/31) |
| B6 FTW-mESCs (EGFP) | ICR | E13.5 | 5 | 3 | 60.0% (3/5) |
| B6 FTW-mESCs (EGFP) | B6 | E13.5 | 21 | 10 | 41.6% (10/21) |

TABLE 2B
Summary of chimera results with mouse FTW-ESCs - adult chimera results.

| | | | | | | Estimated |
|---|---|---|---|---|---|---|
| | | | | | Chimera | degree of |
| | Passage | Blystocyst | Live pups | Coat color | efficiency | coat color |
| Cell line | number | strain | # | chimeras # | % | change % |
| B6 FTW-mESCs | P5-7 | ICR | 11 | 6 | 54.5% | 20%-60% |
| | | | | | (6/11) | |
| B6 FTW-mESCs | P5-7 | B6-Tyr | 25 | 14 | 56.0% | 60%-90% |
| | | | | | (14/25) | |
| B6 FTW-mESCs | P26 | ICR | 8 | 6 | 75.0% | 10%-30% |
| | | | | | (6/8) | |
| B6 mESCs | P5-7 | ICR | 10 | 7 | 70.0% | 40%-70% |
| | | | | | (7/10) | |
| B6 mESCs | P5-7 | B6-Tyr | 16 | 13 | 81.3% | 80%-90% |
| | | | | | (13/16) | |

Distinct from ESCs and EpiSCs, formative EpiLCs are responsive to PGC-LC induction by BMP treatment, which can then form functional gametes after further differentiation in vivo or in vitro. To evaluate the PGC competence of FTW-ESCs, a dual Blimp1-mVenus (BV) /Stella-ECFP (SC) reporter ESC line was converted to FTW-ESCs by culture adaptation. Using a 3D aggregate induction protocol developed for EpiLCs, FTW-ESCs were successfully differentiated into BV+/SC+ cells (FIG. 11C). The role of different signaling pathways (FGF, TGFβ, WNT and LIF) during PGC-LC induction from FTW-ESCs were next investigated by chemical inhibition. Interestingly, the addition of an FGF receptor (FGFR) inhibitor (PD173074) dramatically increased BV+/SC+ population (FIG. 3E, FIG. 3F and FIG. 11D). On the other hand, inhibition of WNT or LIF pathways slightly reduced PGC-LC (BV+/SC+) induction efficiency (FIG. 11D). FGFR inhibition also promoted PGC-LC induction from blastocyst derived FTW-ESCs based on sorting SSEA1+/CD61+ population (FIG. 11E). BV+/SC+ cells isolated from day 2, 4 and 6 during PGC-LC induction showed significant upregulation of PGC markers when compared to uninduced FTW-ESCs (day 0) and BV-/SC-cells (FIG. 3G). Immunofluorescence (IF) analysis confirmed the PGC markers TFAP2C and BLIMP1 were co-expressed at the protein level in day 6 PGC-LCs (FIG. 3H). Moreover, the epigenetic status of induced PGC-LCs were evaluated. IF analysis revealed that 5mC and H3K9me2 levels decreased and H3K27me3 level increased in day 6 PGC-LCs (BLIMP1+ or TFAP2C+ cells) when compared to non-PGC-LCs (BLIMP1− or TFAP2C− cells) (FIGS. 3I-3K).

Collectively, our results demonstrate that in addition to chimera competency, FTW-ESCs are also amenable to direct PGC specification in vitro. Therefore, FTW-ESCs were designated as chimera (Xipatpa in Greek) and PGC dual-competent pluripotent Stem Cells, or XPSCs).

### Example 5. FTW Condition Supports the Derivation of Stable Horse ESCs and Transgene Free iPSCs

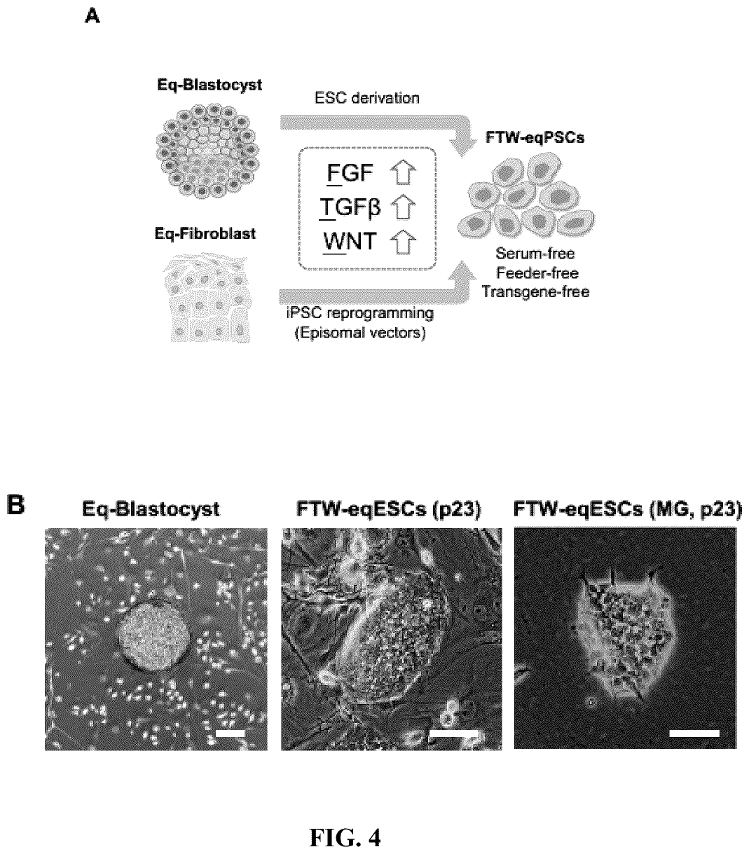

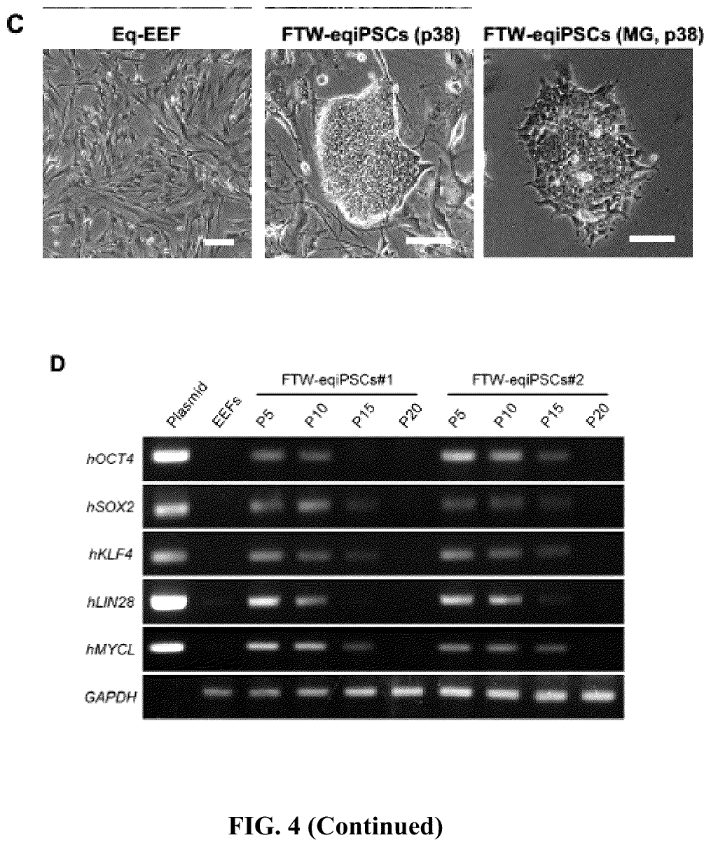

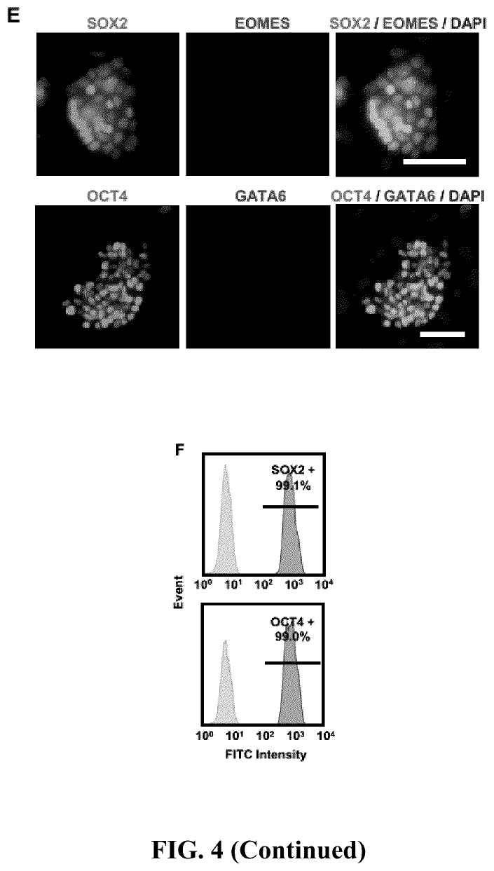

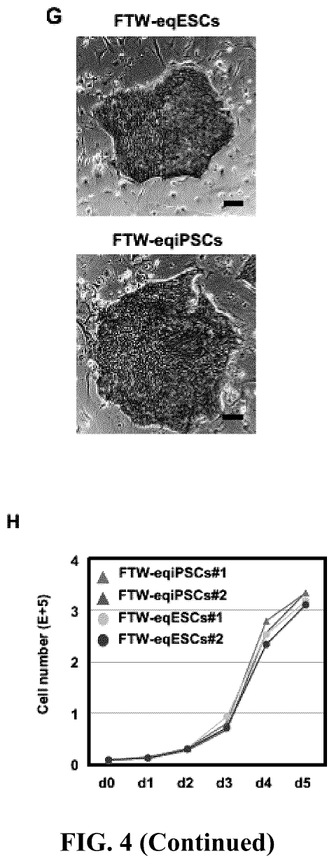

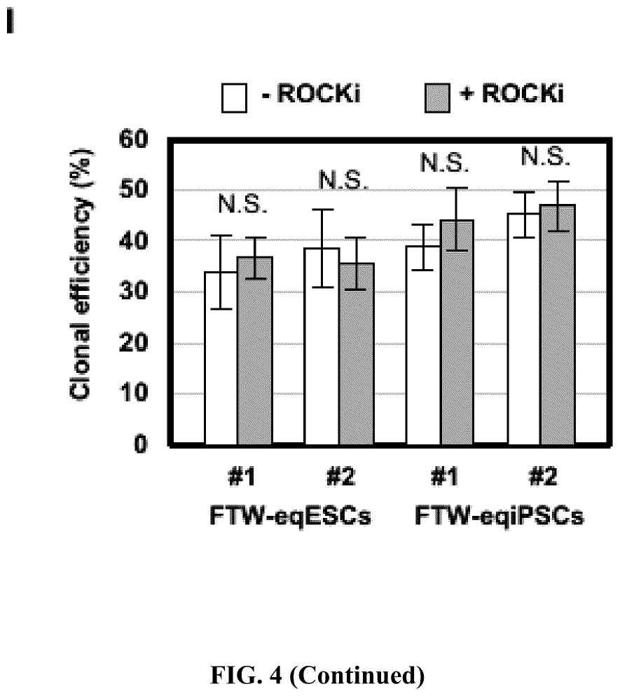

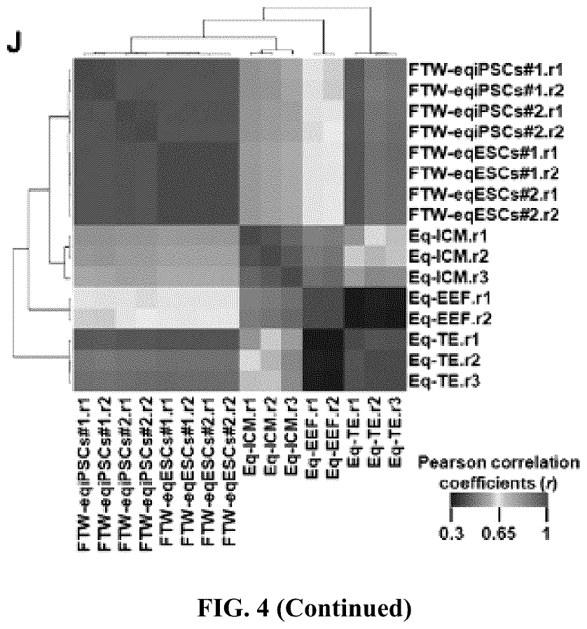

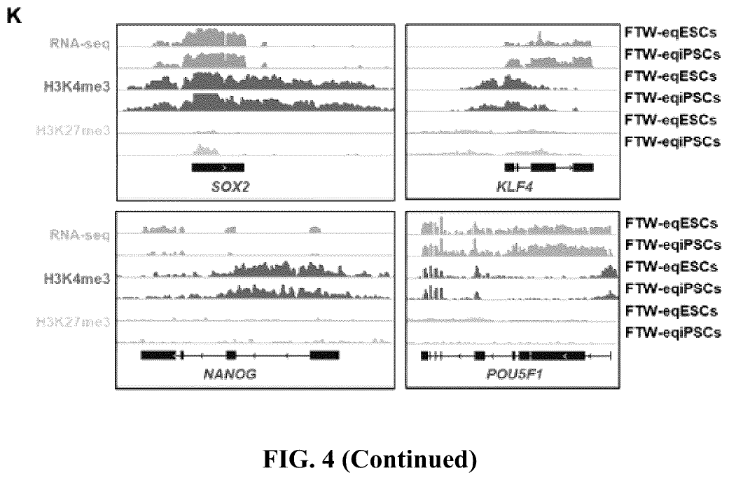

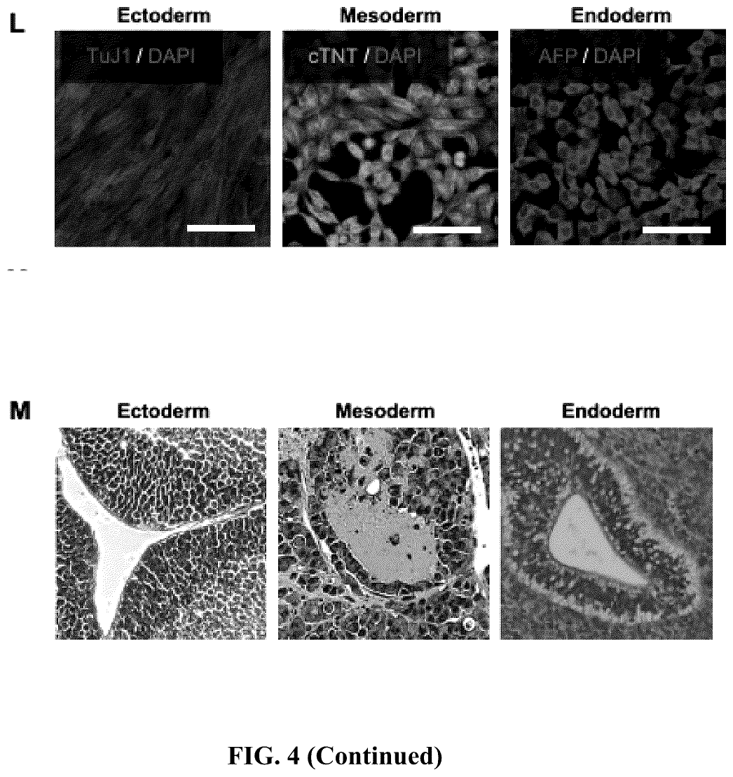

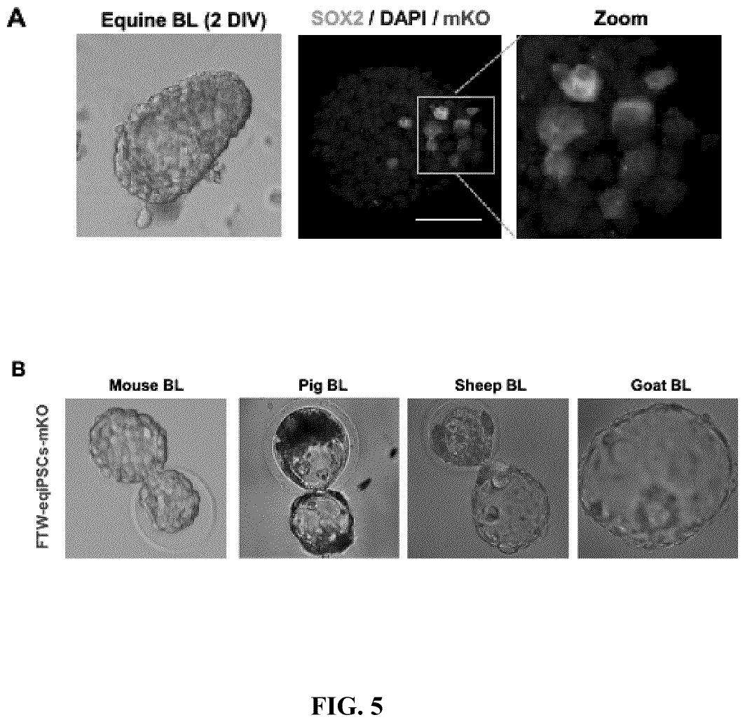

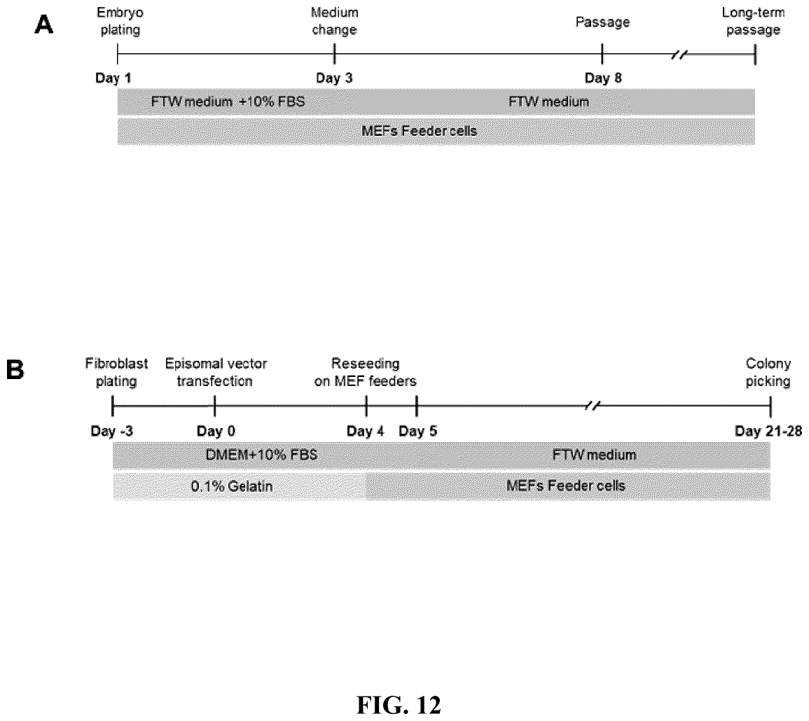

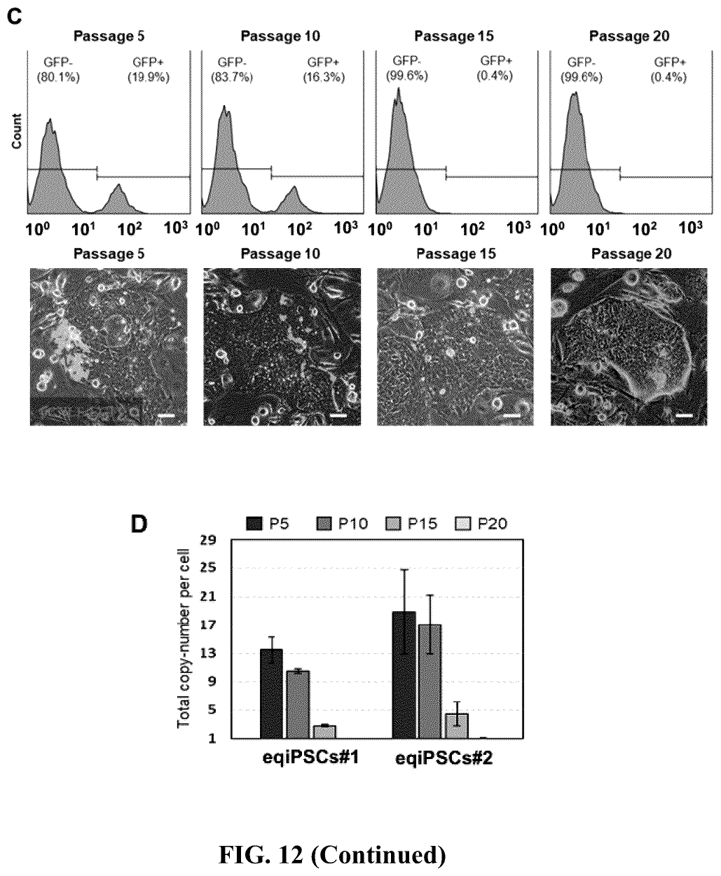

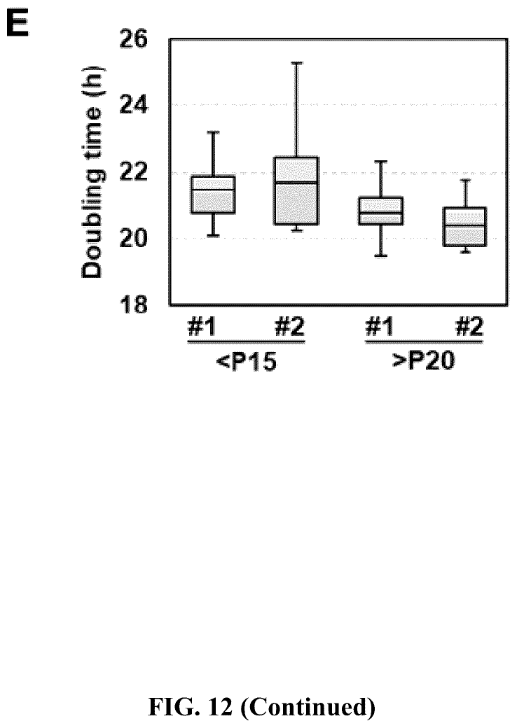

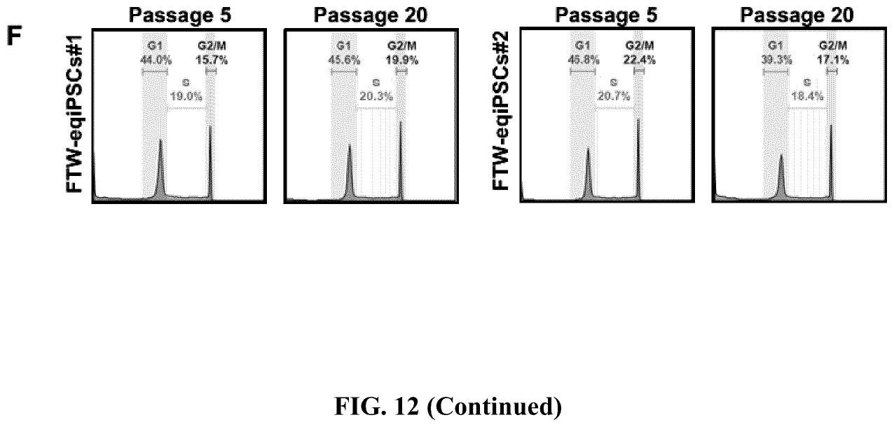

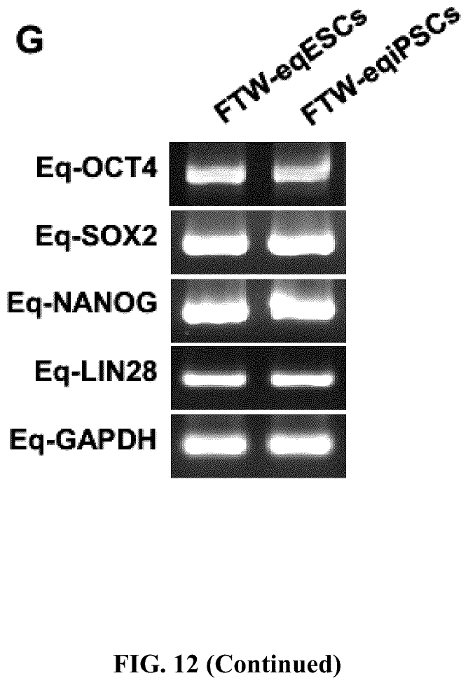

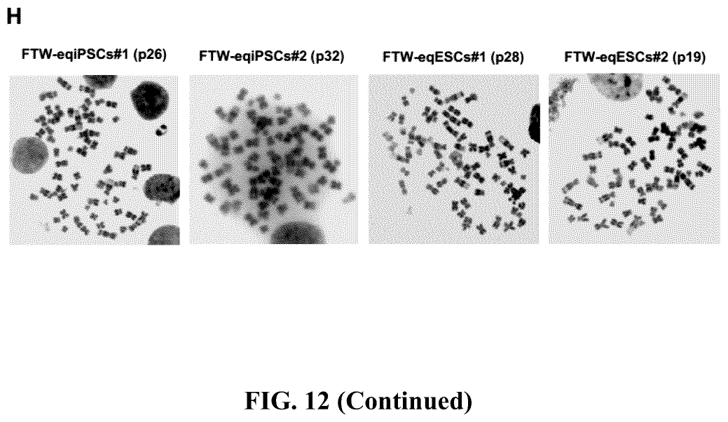

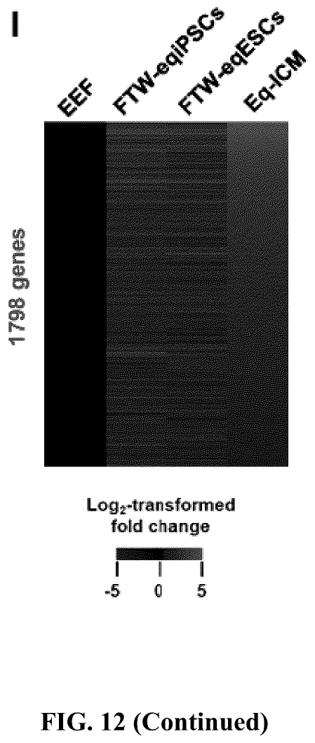

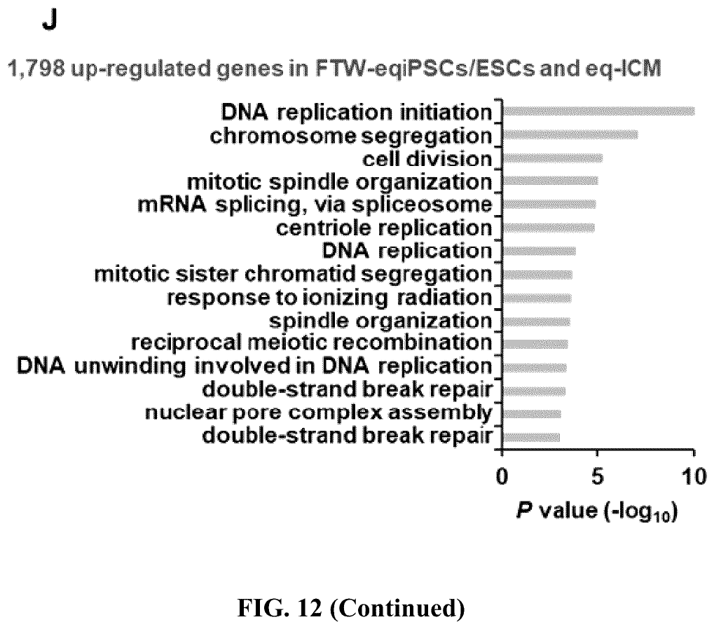

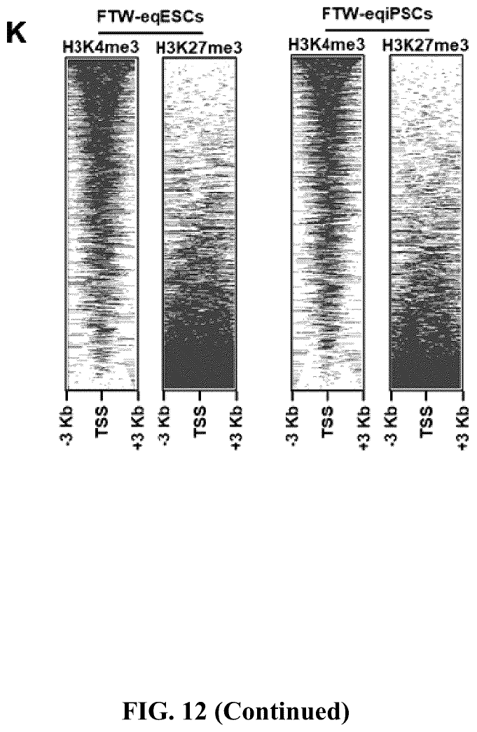

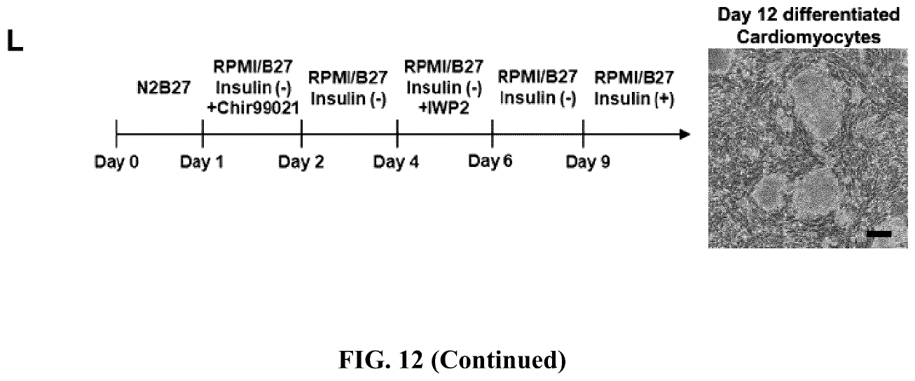

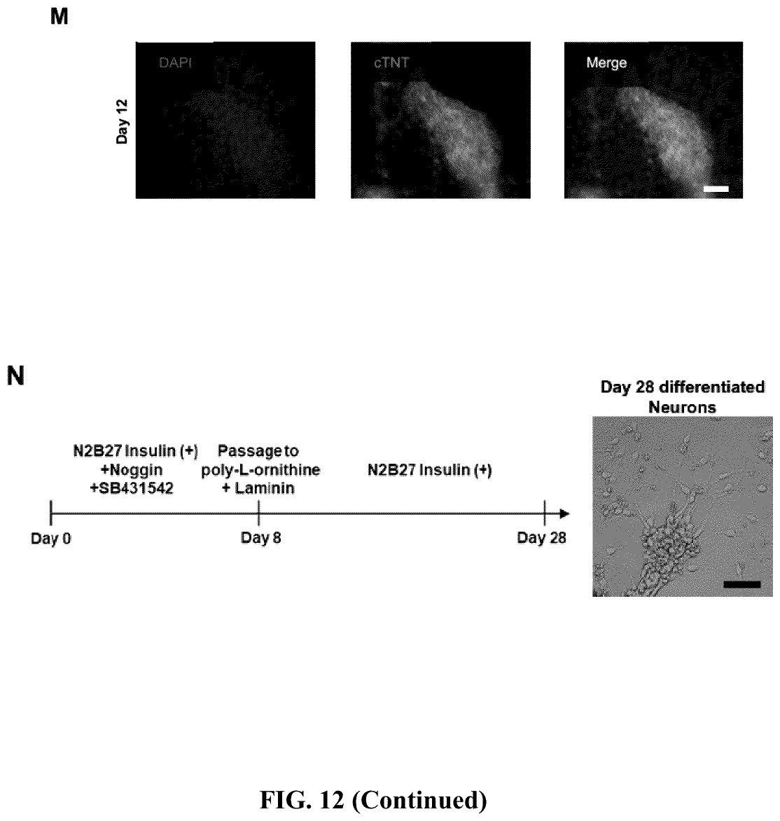

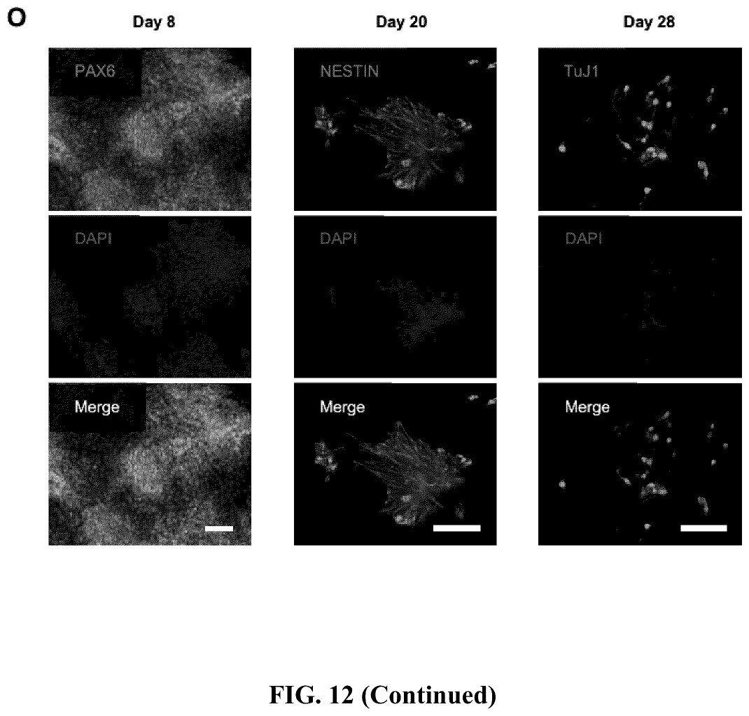

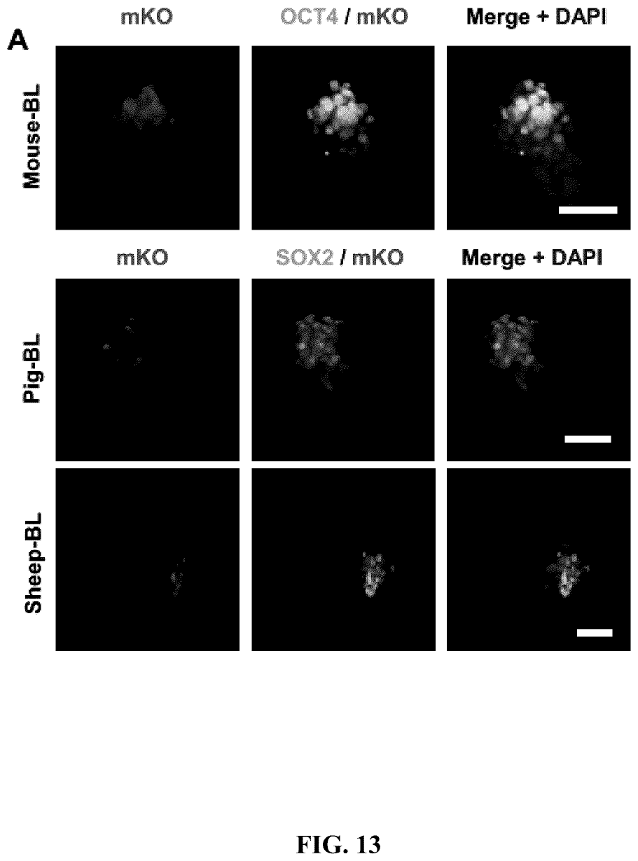

FTW culture was next tested for the derivation of PSCs from species currently lacking stable ESCs. Under FTW conditions, ESCs were successfully derived from horse blastocysts produced by intracytoplasmic sperm injection (ICSI) and somatic cell nuclear transfer (SCNT) (referred to as equine FTW-ESCs, or FTW-eqESCs) (FIG. 4A, FIG. 4B, FIG. 12A, and Table 1). The MEK inhibitor PD0325901 was not needed and supplementation of 10% fetal bovine serum (FBS) during the initial 3 days was beneficial for FTW-eqESC derivation. FTW culture also supported the generation of equine iPSCs (FTW-eqiPSCs) by transfecting equine embryonic fibroblasts (EEFs) with episomal plasmid vectors encoding a combination of human SOX2, KLF4, L-MYC, LIN28, OCT4, and TP53 shRNA (FIG. 4A, FIG. 4C and FIG. 12B). Transgene status of two independent FTW-eqiPSC lines at different passages (P5, P10, P15 and P20) was evaluated by examining 1) the expression of human reprograming factors (FIG. 4D); 2) EGFP expression from a co-transfected pCXLE-EGFP episomal vector (FIG. 12C); and 3) total copy number of episomal vectors per cell (FIG. 12D). It was found that after long term passage (P20) both FTW-eqiPSC lines had acquired transgene-free status with negligible changes in doubling time and cell cycle profiles (FIG. 12E and FIG. 12F).

FTW-eqPSCs could be maintained on MEFs or in a feeder-free condition (Matrigel, MG) (FIG. 4B and FIG. 4C), expressed pluripotency genes at both transcript and protein levels (FIG. 4E, FIG. 4F and FIG. 12G), and exhibited alkaline phosphatase activity (FIG. 4G). FTW-eqPSCs could be recovered efficiently after multiple freeze/thaw cycles, maintained stable growth kinetics (FIG. 4H) and normal (n=64) diploid chromosome numbers (FIG. 12H) after long term culture. Similar to mouse XPSCs, FTW-eqPSCs could be passaged at clonal density without ROCK inhibitor treatment (FIG. 4I).

### Example 6. FTW-eqPSCs Exhibit Molecular and Functional Features of Pluripotency

RNA-seq was performed on FTW-eqESCs, FTW-eqiPSCs, EEFs, equine inner cell mass (eq-ICM), and trophectoderm (eq-TE). Unsupervised hierarchical clustering and correlation (UHC) analysis showed that FTW-eqESCs and FTW-eqiPSCs clustered closer with eq-ICMs than EEFs and eq-TEs (FIG. 4J). Pair-wise comparisons of EEF transcriptome with those of FTW-eqESCs, FTW-eqiPSCs, and eq-ICM identified 1,798 commonly up-regulated genes in eqESCs, FTW-eqiPSCs and eq-ICMs (FIG. 12I). GO analysis revealed that many enriched terms were related to cell division and proliferation, e.g., DNA replication initiation, chromosome segregation, and cell division (FIG. 12J). ChIP-seq analysis was also performed and it was found that FTW-eqESCs and FTW-eqiPSCs showed similar global H3K4me3 and H3K27me3 profiles (FIG. 12K). The analysis revealed enriched H3K4me3 marks and decreased levels of H3K27me3 within the promoter regions of several pluripotency related transcription factors, e.g., SOX2, KLF4, NANOG, and POU5F1, which correlated with gene expression (FIG. 4K).

To functionally evaluate the pluripotency of FTW-eqPSCs, in vitro (embryoid body) and in vivo (teratoma) differentiation assays were performed. IF and histological analysis demonstrated that FTW-eqPSCs differentiated into cells and tissues from all three germ lineages: ectoderm, mesoderm and endoderm (FIG. 4L and FIG. 4M). Also generated were beating cardiomyocytes (FIG. 12L and FIG. 12M) and neurons (FIG. 12N and FIG. 12O) from FTW-eqiPSCs via directed differentiation, indicating they are responsive to inductive cues and competent for multilineage commitment.

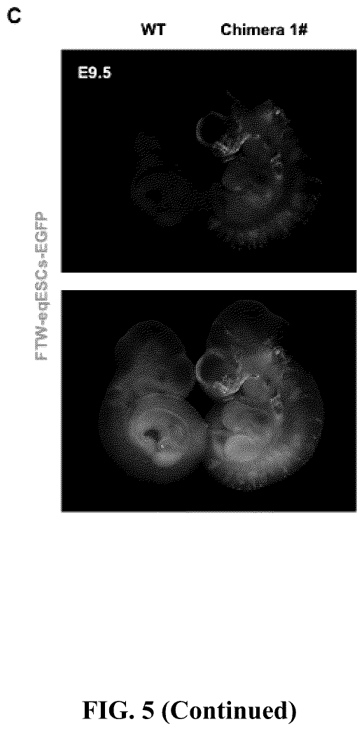

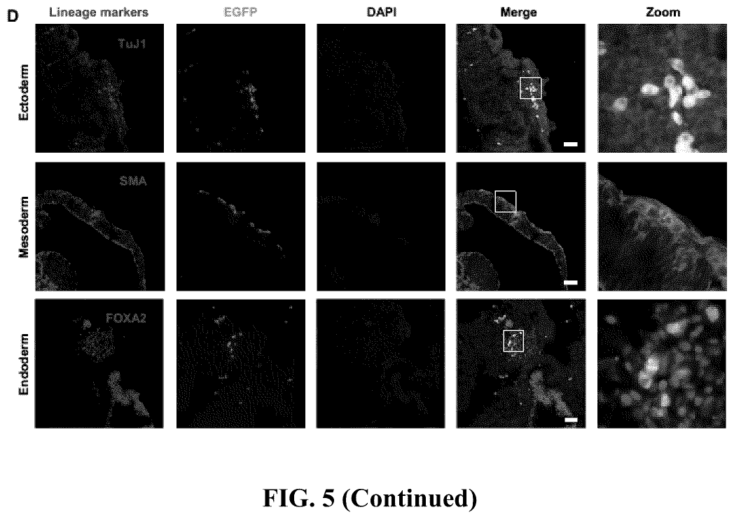

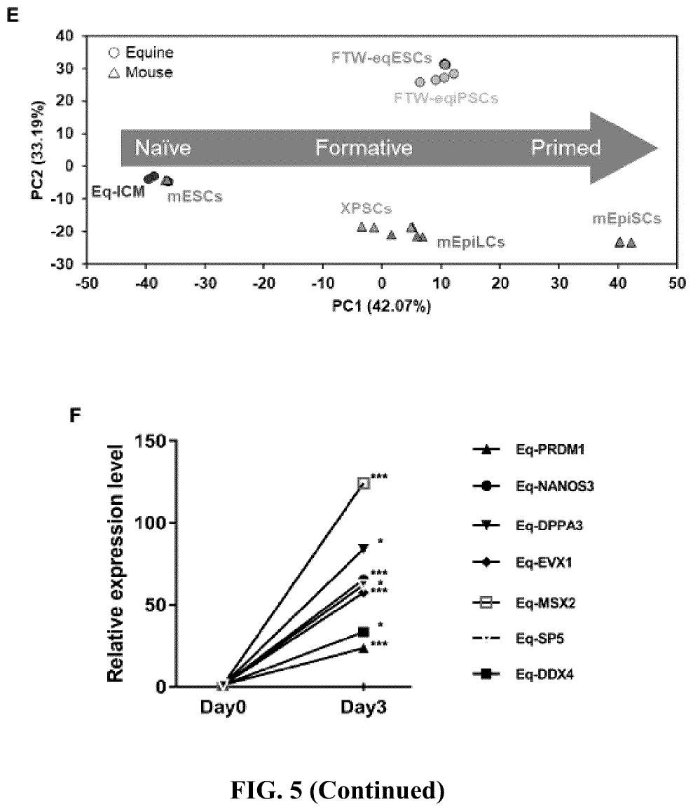

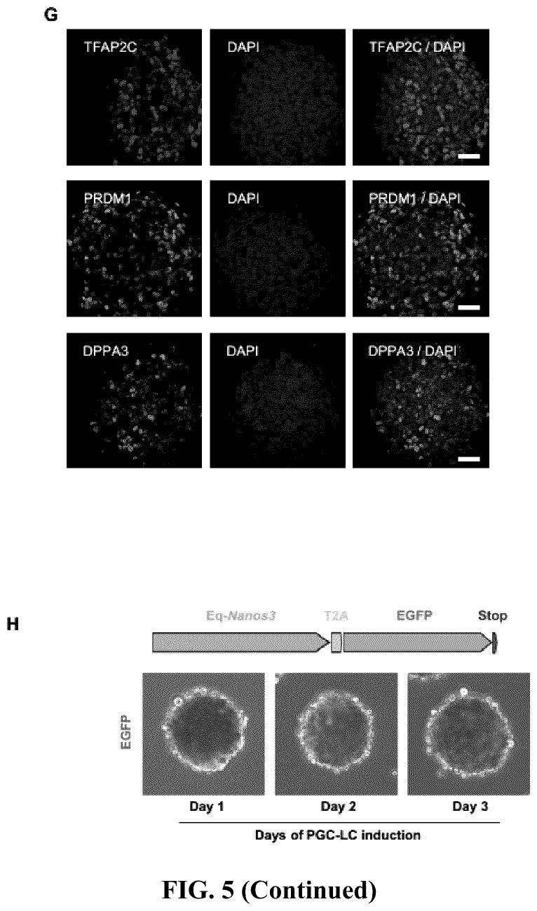

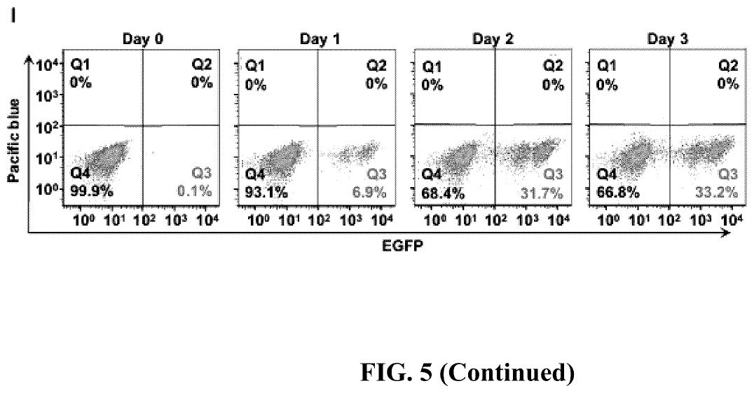

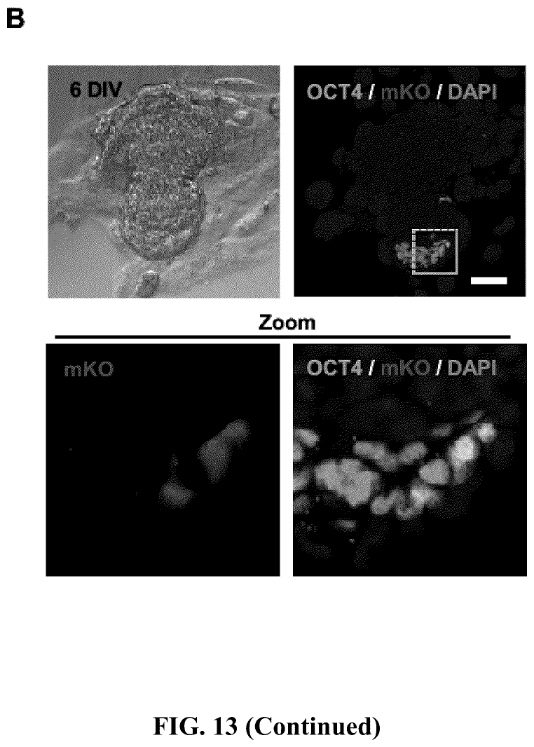

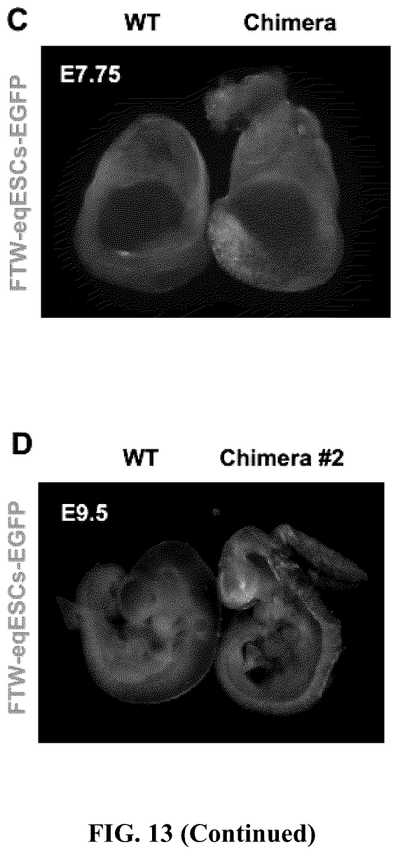

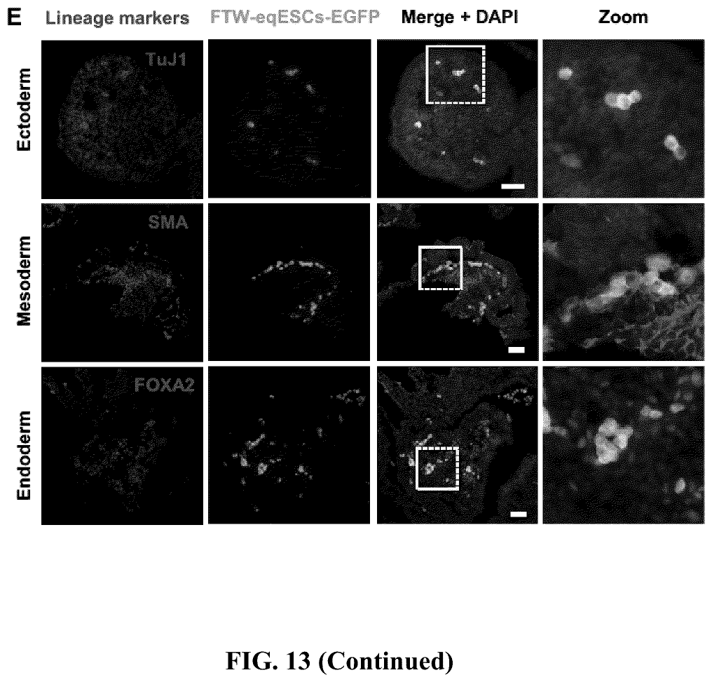

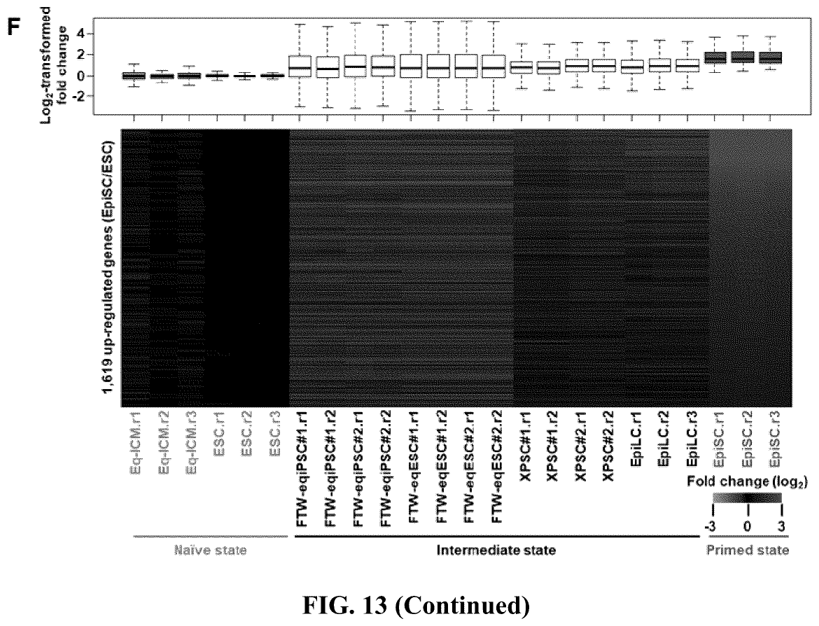

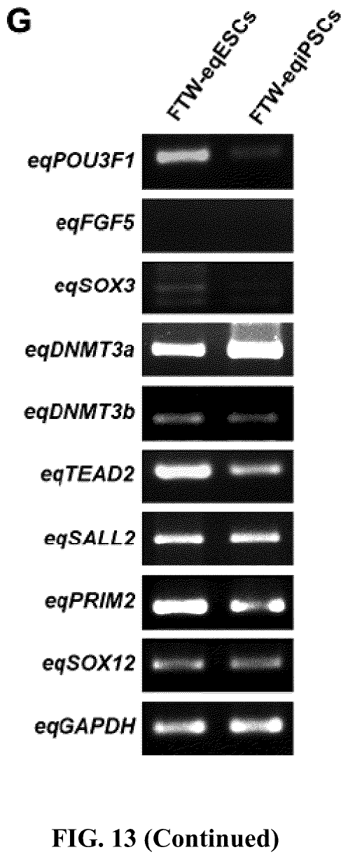

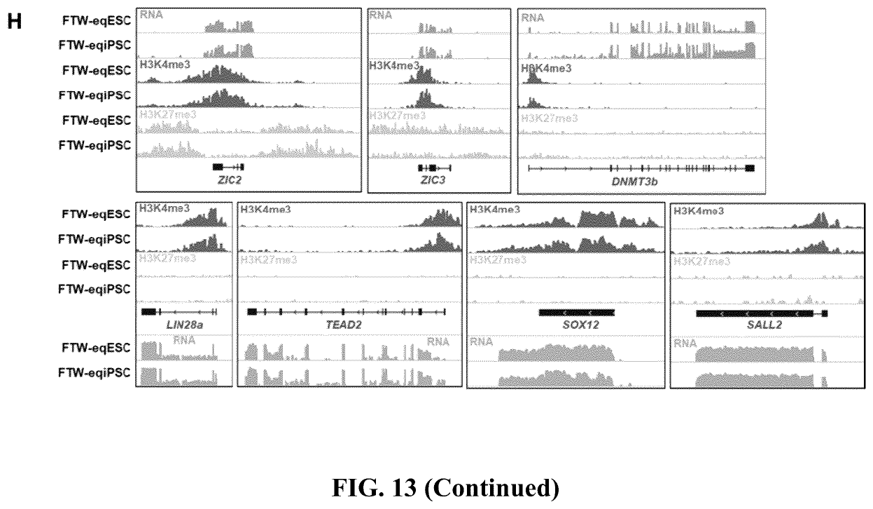

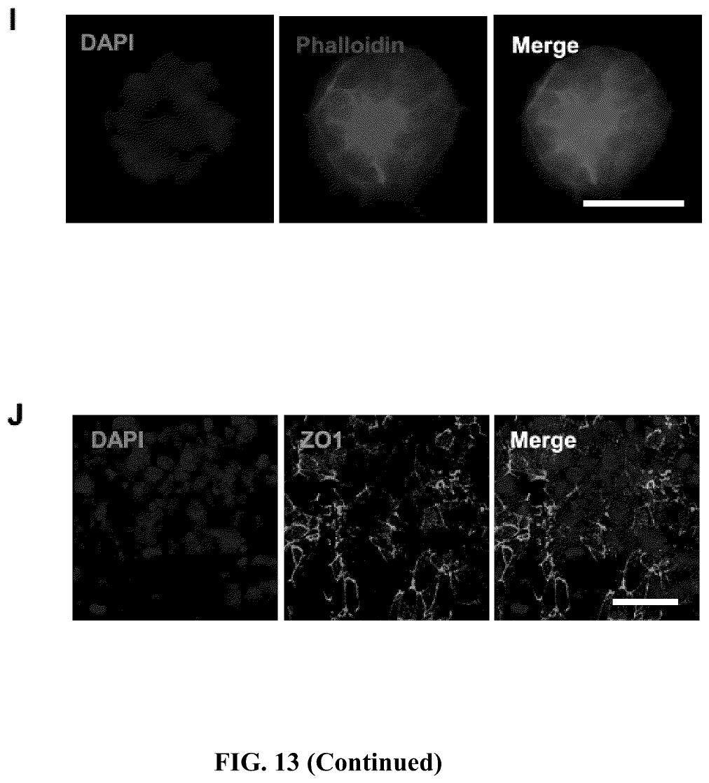

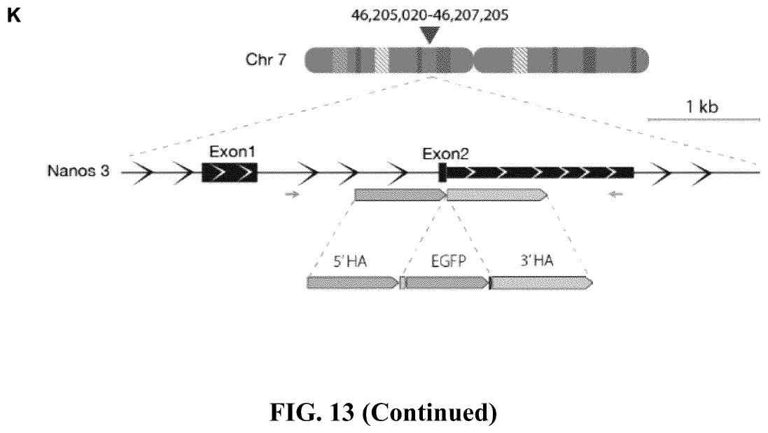

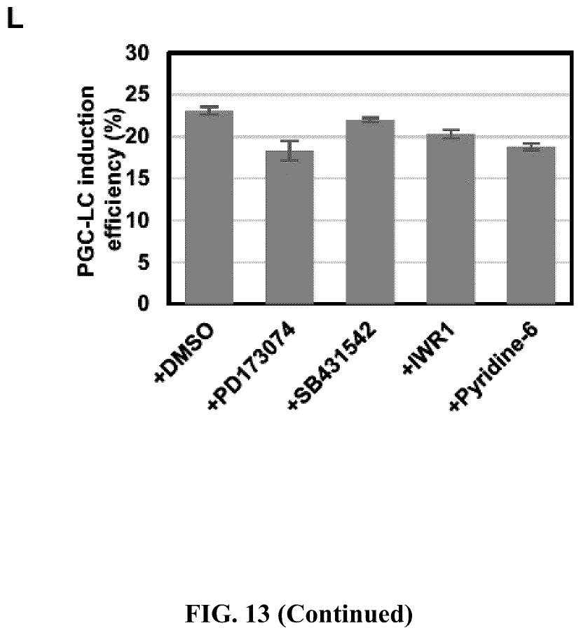

A more stringent test for pluripotency is chimera formation, which measures whether donor PSCs can extensively colonize all germ layer tissues after being introduced into pre-implantation host embryos. Due to logistic difficulties in performing such experiments in horses, the ability of FTW-eqPSCs to integrate into the ICM of a blastocyst was first examined. Monomeric Kusabira-Orange (mKO) labeled FTW-eqiPSCs were injected into horse blastocysts and cultured in vitro for two days before analyzing chimeric ICM formation by fluorescence microscopy and IF. Two days post-injection, FTW-eqiPSCs were found incorporated in the ICMs of 6 out of 6 (100%) injected horse blastocysts and expressed the pluripotency transcription factor SOX2 (FIG. 5A and Tables 3A-3C). Interspecies ICM incorporation of mKO-labeled FTW-eqiPSCs to mouse, sheep, goat and pig blastocysts following blastocyst or morula injections were also examined. Consistently, FTW-eqiPSCs efficiently incorporated into host ICMs and expressed OCT4 (mouse) and SOX2 (sheep and pig) (FIG. 5B, FIG. 13A and Tables 3A-3C). Next, it was determined whether FTW-eqiPSCs could survive and retain in mouse post-implantation epiblast by culturing FTW-eqPSCs-injected mouse blastocysts for an additional 6 days in vitro extended culture following an established protocol. FTW-eqiPSCs could be detected within mouse epiblast layer, albeit at a lower rate than ICM (22.2%, 6/27), and stained positive for OCT4 (FIG. 12B).

ICM and/or epiblast integration is an indicator of chimera potential but not the definitive proof for chimera competency. Next, embryo transfers using mouse blastocysts injected with EGFP-labeled FTW-eqESCs were performed. Interestingly, a number of EGFP+ cells in (5/18, 27.8%) E7.75 and (4/24, 16.7%) E9.5 mouse embryos (FIG. 5C, FIG. 12C, FIG. 12D and Tables 3A-3C) were detected. Following embryo sectioning and co-staining with lineage specific markers (Ectoderm, TUJ1; Endoderm, FOXA2; Mesoderm, SMA), it was found that FTW-eqESCs differentiated into all three germ lineages in E9.5 mouse embryos (FIG. 5D and FIG. 12E). These results demonstrate the ability of FTW-eqESCs to contribute to multilineage chimera formation in mice.

Collectively, these results demonstrate that FTW-eqPSCs exhibit molecular and functional hallmarks of pluripotency.

TABLE 3A
Summary of chimera formation results with horse FTW-iPSCs and
FTW-ESCs-ICM incorporation

| | | Injected | Injected | Develop- | ICM |
|---|---|---|---|---|---|
| | Host | Morula | Blastocyst | ment | contribution |
| Cell line | Species | #s | # | Rate % | Rate % |
| FTW-eqiPSCs | Equine | — | 6 | 100% | 83.3% |
| (mKO) | | | | (6/6) | (5/6) |
| FTW-eqiPSCs | Sheep | 39 | — | 66.7% | 88.5% |
| (mKO) | | | | (26/39) | (23/26) |
| FTW-eqiPSCs | Goat | 59 | — | 74.6% | 72.7% |
| (mKO) | | | | (44/59) | (32/44) |
| FTW-eqiPSCs | Pig | 83 | — | 57.8% | 43.8% |
| (mKO) | | | | (48/83) | (21/48) |
| FTW-eqiPSCs | Mouse | — | 58 | 98.3% | 67.2% |
| (mKO) | | | | (57/58) | (39/58) |

TABLE 3B
Summary of chimera formation results with horse
FTW-iPSCs and FTW-ESCs - In vitro embryo culture

| | | | | | | Epiblast |
|---|---|---|---|---|---|---|
| | | | | ICM | In vitro | layer |
| | Host | Host | Injected | contribution | cultured | contribution |
| Cell line | Species | Strain | Blastocysts # | rate % | Blastocysts # | rate % |
| FTW-eqiPSCs | Mouse | ICR | 58 | 67.2% | 27 | 22.2% (6/27) |
| (mKO) | | | | (39/58) | | |

TABLE 3C
Summary of chimera formation results with horse FTW-iPSCs
and FTW-ESCs-In vivo embryo transfer.

| | | | | Total | EGFP+ | |
|---|---|---|---|---|---|---|
| | Host | Host | Dissection | embryos | Chimerias | |
| Cell line | Species | Strain | stage | # | # | Chimeras % |
| FTW- | Mouse | ICR | E7.75 | 18 | 5 | 27.8% (5/18) |
| eqESCs | | | | | | |
| (EGFP) | | | | | | |
| FTW- | Mouse | ICR | E9.5-10.5 | 24 | 4 | 16.7% (4/24) |
| eqESCs | | | | | | |
| (EGFP) | | | | | | |

### Example 7. FTW-eqPSCs Harbor Formative Pluripotency Features

Next, it was determined whether FTW-eqPSCs also retained mouse formative pluripotency features. To this end, the transcriptome profiles of eq-ICMs and FTW-eqPSCs were compared with mouse ESCs, XPSCs, EpiLCs, and EpiSCs. It was found that eq-ICMs clustered together with mouse ESCs, and FTW-eqPSCs aligned with mouse XPSCs and EpiLCs along the naïve-formative-primed axis (FIG. 5E). This analysis also identified 1,619 mouse primed pluripotency related genes (up-regulated genes in mouse EpiSCs versus ESCs) and examined their expression levels in equine (eq-ICMs and FTW-eqPSCs) and mouse (ESCs, XPSCs, EpiLCs, and EpiSCs) samples. It was found that the expression patterns of these genes in FTW-eqPSCs were intermediate of naïve (eq-ICMs and mouse ESCs) and primed (mouse EpiSCs) cells, and more similar to mouse XPSCs and EpiLCs (FIG. 12F). Using horse-specific primers, it was confirmed that the expression of some mouse formative markers in FTW-eqPSCs via RT-PCR analysis (FIG. 12G). From the ChIP-seq datasets, the enrichment of H3K4me3 marks and decreased H3K27me3 levels around the TSS of formative pluripotency related genes in FTW-eqPSCs (FIG. 12H) was also observed. Moreover, FTW-eqPSCs could form rosette-like structures and tight junctions similar to mouse XPSCs (FIG. 12I and FIG. 12J).

It was also examined whether FTW-eqPSCs were permissive for direct PGC-LC induction. Similar to mouse XPSCs, it was found that FTW-eqESCs could also respond to BMP signaling and gave rise to PGC-LCs as evidenced by: 1) qPCR analysis demonstrating the expression levels of several PGC markers were significantly elevated after 3 days of induction with BMP4 (FIG. 5F), and 2) IF analysis indicating that several PGC markers (PRDM1, TFAP2C and DPPA3) were also expressed at the protein level (FIG. 5G). To confirm PGC-LCs could be directly induced from FTW-eqESCs, a fluorescent NANOS3 reporter line was generated via CRISPR/Cas9 insertion of a T2A-EGFP sequence into the endogenous NANOS3 gene locus immediately before the stop codon (FIG. 5H and FIG. 12K). One homozygous NANOS3-EGFP clone was selected to confirm PGC-LC induction, and observed EGFP signal after one day of BMP4 treatment. The number of EGFP+ cells continued to increase on days 2 and 3 (FIG. 5H). FACS analysis was also performed, and results that found 6.9%, 31.7% and 33.2% of cells were EGFP+ after 1, 2 and 3 days of induction, respectively (FIG. 5I). Interestingly, different from mouse XPSCs, treatment with FGFR inhibitor PD173074 did not enhance PGC-LC induction efficiency from FTW-eqESCs (FIG. 12L).

In sum, similar to mouse XPSCs, FTW-eqPSCs are responsive to PGC specification in vitro and can contribute to chimera formation in mouse embryos in vivo. Accordingly, FTW-eqPSCs are hereafter referred to as equine XPSC (eqXPSC).

### Example 8. Generation and Characterization of Human FTW-iPSCs

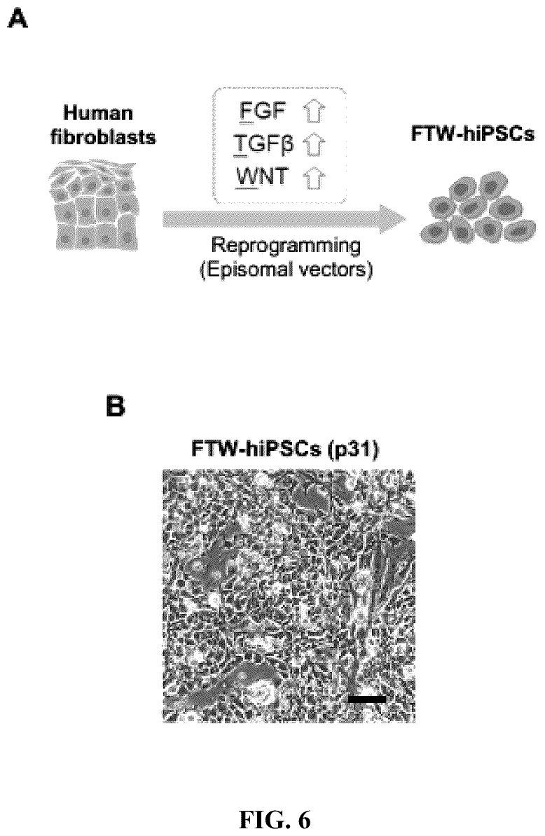

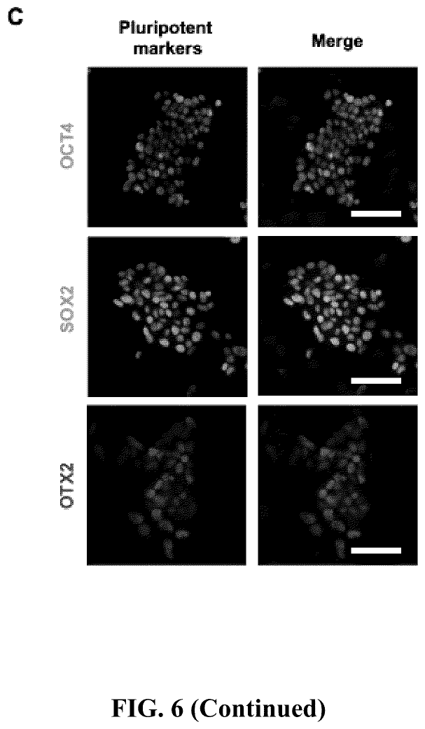

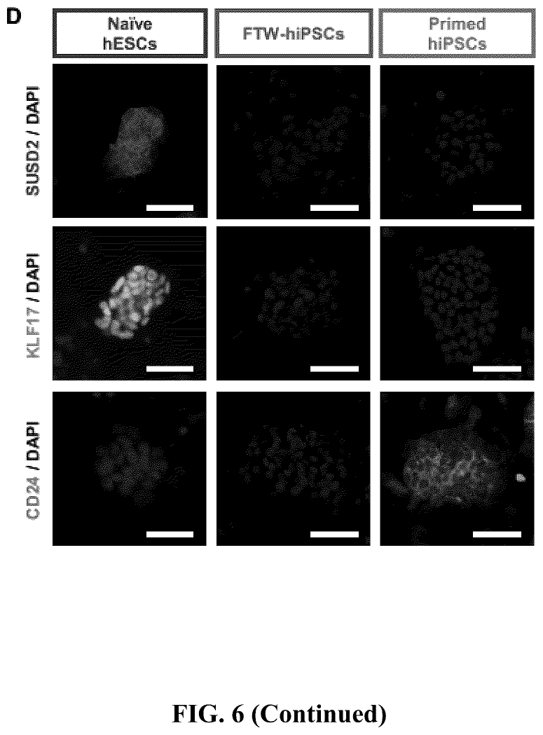

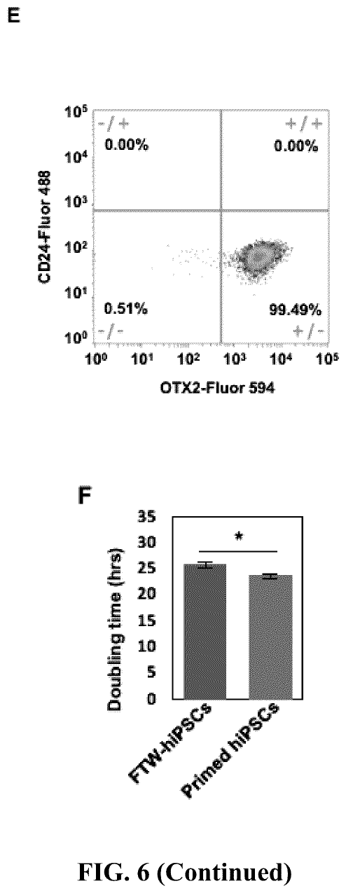

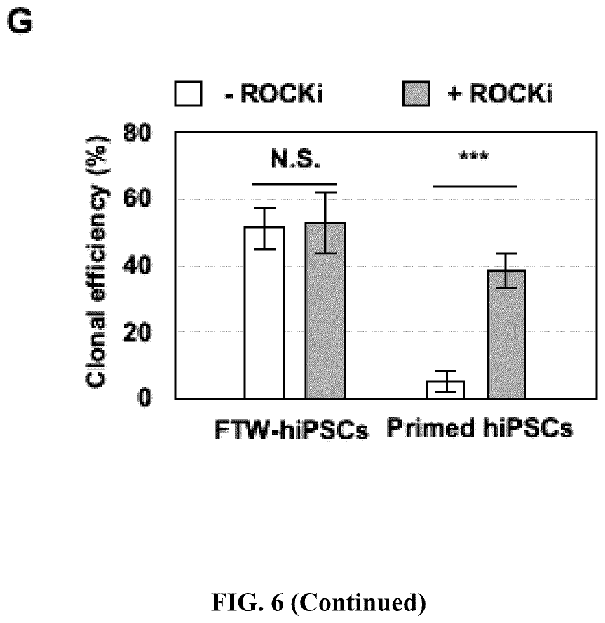

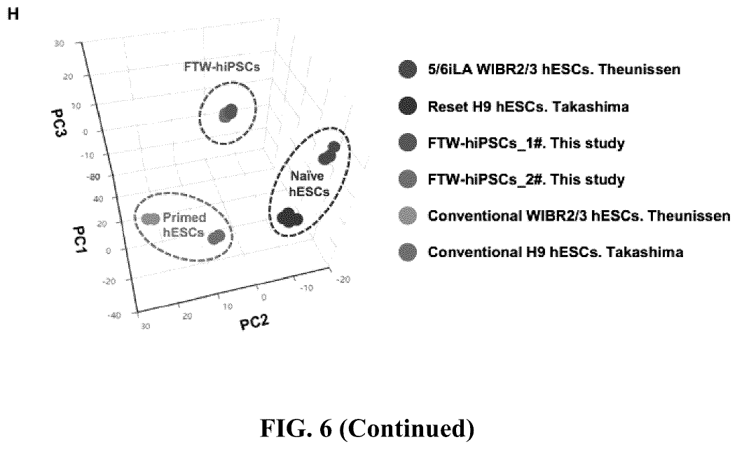

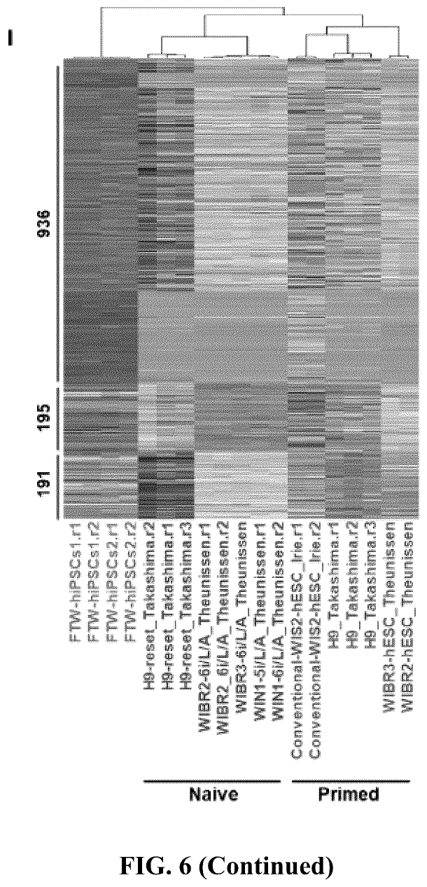

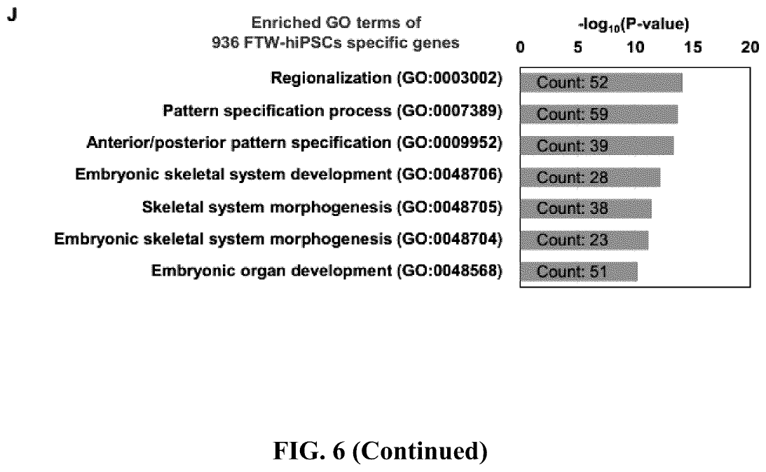

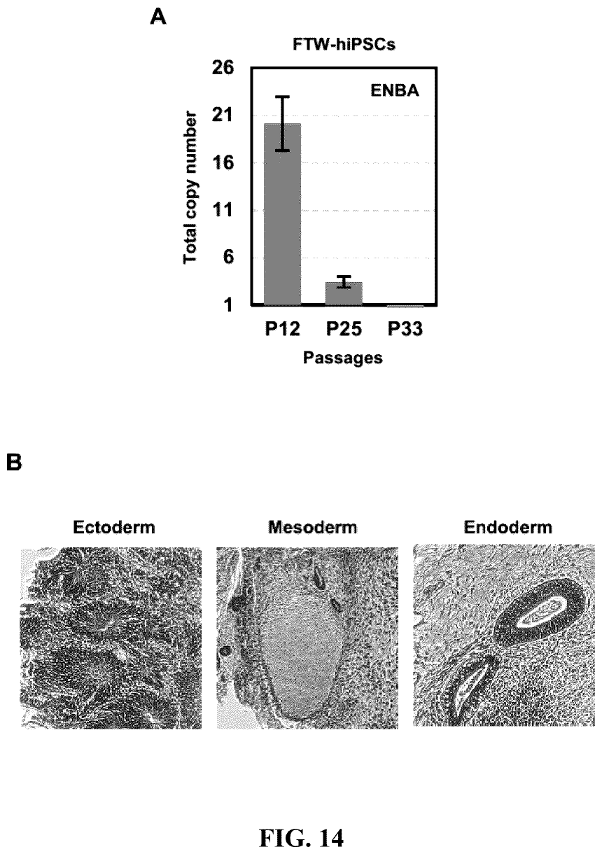

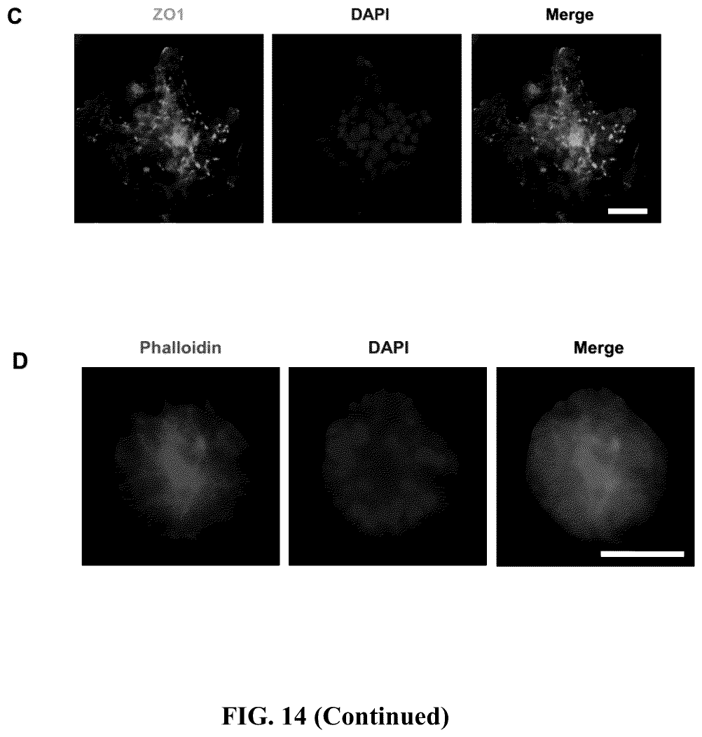

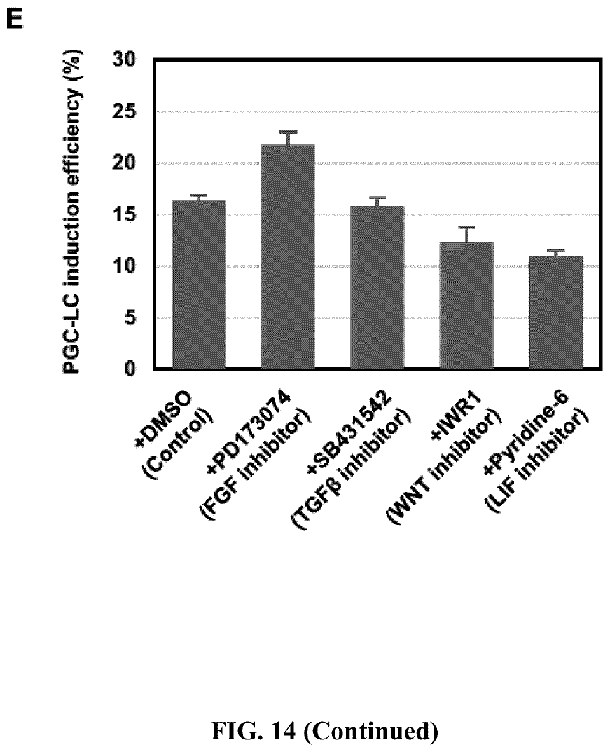

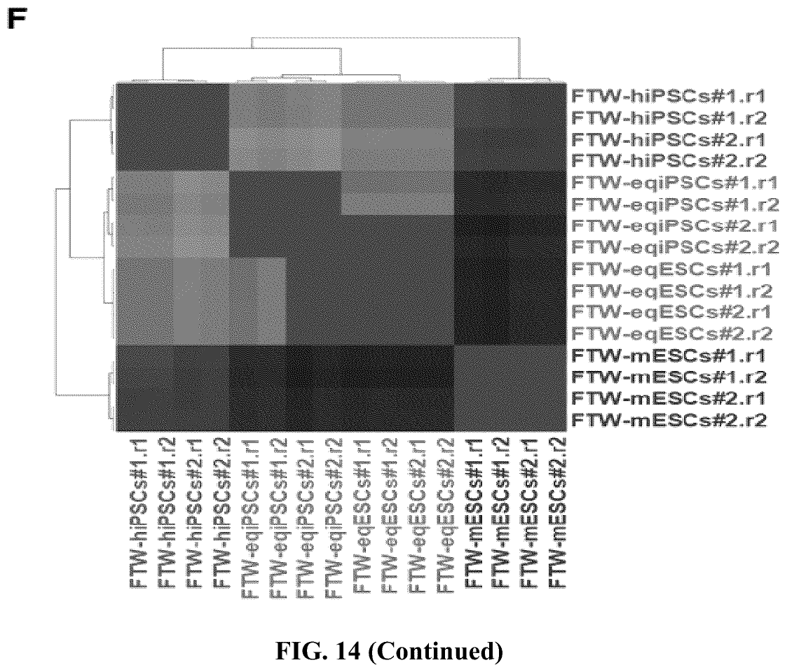

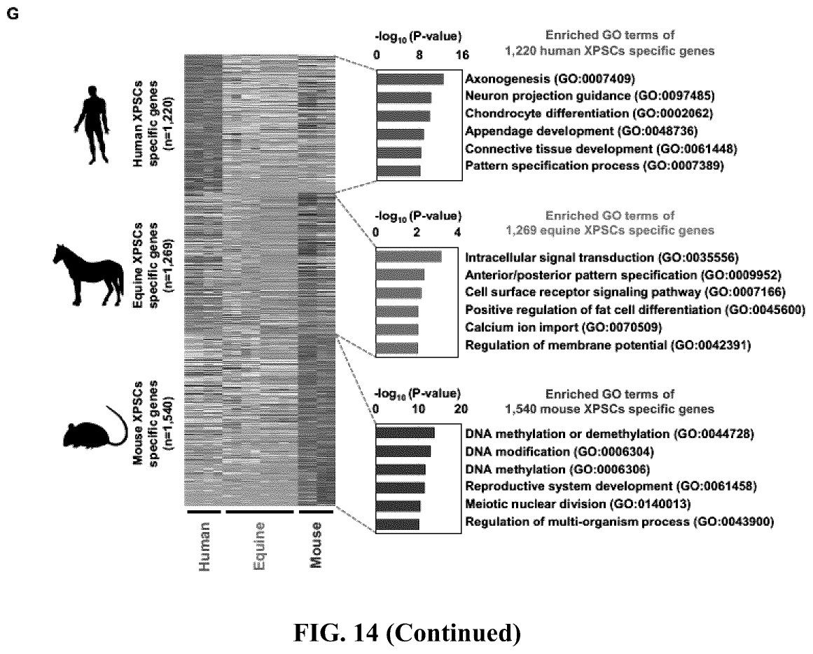

Recently, an iPSC line was generated under the FTW condition (FAC) from human foreskin fibroblasts (HFFs), which demonstrated chimeric competency in E21-28 pig embryos (designated as FTW-hiPSCs #1). However, FTW-hiPSCs #1 have not been characterized in details and it remains unknown whether they resemble mouse XPSCs grown in the same condition. Here, another FTW-iPSC line was generated from HFFs with episomal plasmid vectors (designated as FTW-hiPSCs #2) (FIG. 6A and FIG. 6B) and extensive characterization was performed. FTW-hiPSCs expressed pluripotency markers SOX2 and OCT4 (FIG. 6C) (Wu et al., 2017). Interestingly, FTW-hiPSCs stained negative for KLF17, SUSD2 and CD24, well-recognized human naïve (KLF17 and SUSD2) and primed (CD24) pluripotency markers (FIG. 6D and FIG. 6E), but homogeneously expressed the formative pluripotency marker OTX2 (FIG. 6C and FIG. 6E). FTW-hiPSCs showed a slightly longer doubling time when compared to primed hiPSCs (FIG. 6F). In contrast to primed hiPSCs and similar to mouse and horse XPSCs, FTW-hiPSCs could be passaged at clonal density without treating with a ROCK inhibitor (FIG. 6G). After 33 passages, the episomal vectors sequence could no longer be detected, demonstrating that FTW-hiPSCs can be maintained transgene-free (FIG. 14A). In addition, similar to FTW-hiPSCs_#1, FTW-hiPSCs_#2 could also form teratomas comprised of tissues from all three germ lineages (FIG. 14B).

RNA-seq analysis using both FTW-hiPSC lines was next performed, and their transcriptomes with naïve and primed hESCs (Takashima et al., Cell, 158, 1254-1269, 2014; Theunissen et al., Cell Stem Cell, 15, 471-487, 2014) were compared. A 3D PCA plot showed that FTW-hiPSCs clustered tightly together and were separate from both naïve and primed hESCs (FIG. 6H). RNA-seq analysis identified 936 genes specifically upregulated in FTW-hiPSCs (FIG. 6I), which were enriched in GO terms related to regionalization, anterior/posterior pattern specification, embryonic organ development, among others (FIG. 6J).

These results demonstrate that, similar to mouse XPSCs, FTW-hiPSCs are in a pluripotency state distinct from naïve and primed states.

### Example 9. FTW-hiPSCs Harbor Formative Pluripotency Features

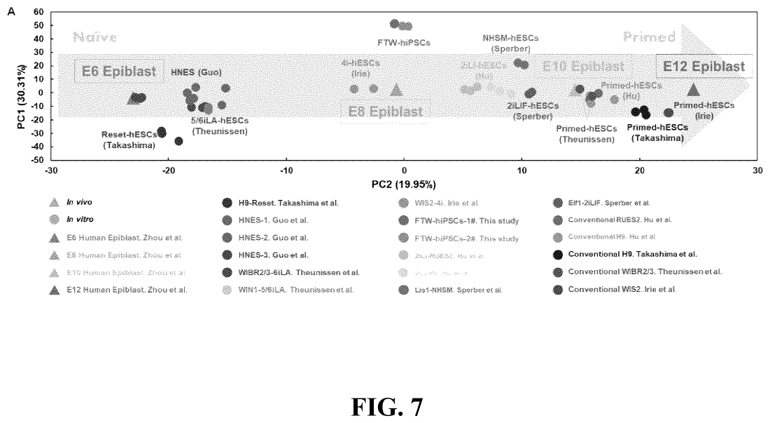

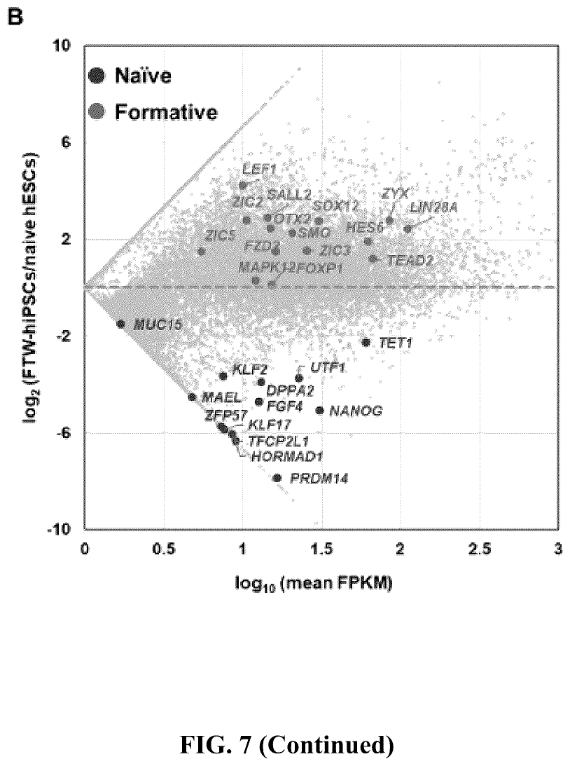

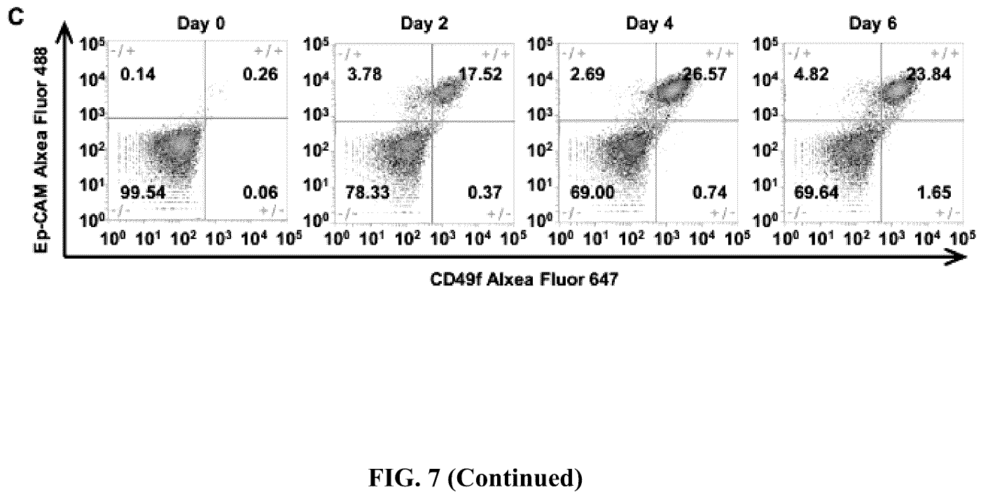

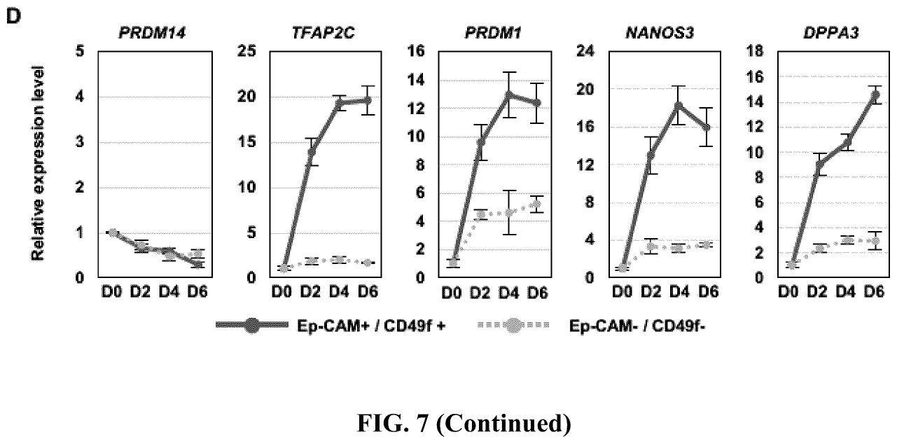

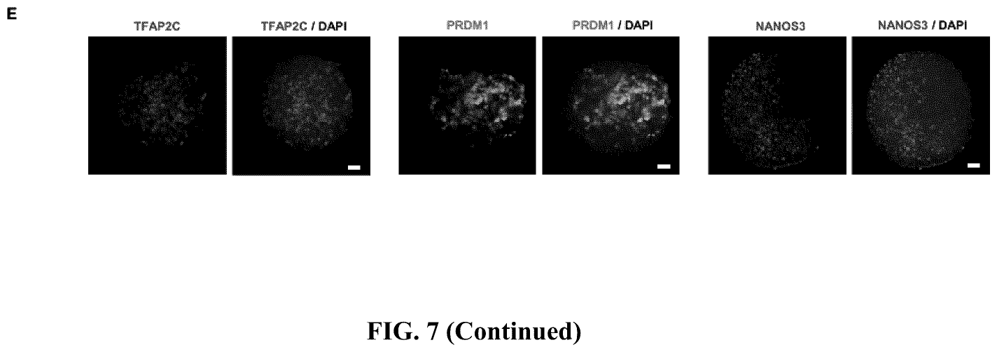

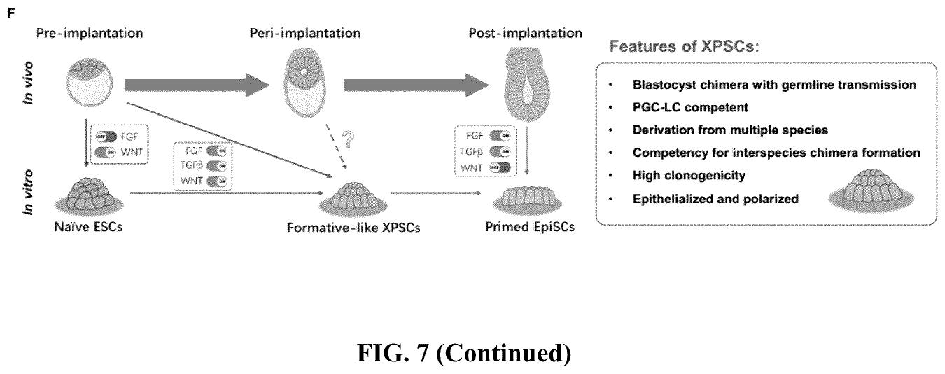

Next, FTW-hiPSCs' transcriptomes were compared with published RNA-seq datasets of cultured human embryos (F. Zhou et al., Nature, 572, 1-27, 2019), naïve (Reset-hESCs, HNES, 4i-hESCs, 2iLI-hESCs, 2iLIF-hESCs and NHSM-hESCs) and primed hESCs (Guo et al., Stem cell Reports, 6, 437-446, 2016; Hu et al., Science Advances, 6, eaaz0298, 2020; Irie et al., Cell, 160, 253-268, 2015; Sperber et al., Nature Cell Biology, 17, 1523-1535, 2015; Takashima et al., Cell, 158, 1254-1269, 2014; Theunissen et al., Cell Stem Cell, 15, 471-487, 2014). PCA analysis revealed that naïve hESCs (Reset-hESCs, 5/6iLA-hESCs and HNES), FTW-hiPSCs and primed hESCs clustered closer to human E6, E8 and E10-12 epiblasts, respectively (FIG. 7A), which indicates that FTW-hiPSCs represent an intermediate pluripotent state resembling the formative epiblast (Smith, Development, 144, 365-373, 2017). Furthermore, many formative pluripotency related genes were upregulated and naïve pluripotency related genes were downregulated in FTW-hiPSCs when compared to naïve hESCs (FIG. 7B). Moreover, similar to mouse and horse XPSCs, FTW-hiPSCs could also form rosette-like structures and tight junctions (FIG. 14C and FIG. 14D). Interestingly, consistent with several recent studies (Guo et al., Development, 144, 2748-2763, 2017; Huang et al., Cell Stem Cell, 15, 410-415, 2014; Nakamura et al., Nature, 537, 57-62, 2016; Pastor et al., Cell Stem Cell, 0, 323-329, 2016), the transcriptomes of hESCs grown in some naïve conditions (4i-hESCs (Irie et al., 2015), 2iLI-hESCs (Hu et al., Science Advances, 6, eaaz0298, 2020), 2iLIF-hESCs and NHSM-hESCs (Sperber et al., Nature Cell Biology, 17, 1523-1535, 2015)) clustered closer to E8 or between E8-10 human epiblasts, suggesting their intermediate pluripotency status (FIG. 7A).

Next, whether FTW-hiPSCs could directly respond to BMP signaling to form PGC-LCs in vitro was investigated. Two surface markers: EpCAM and CD49f (integrin a6), which were shown to selectively label human PGC-LCs (Sasaki et al., 2015), were relied on for purifying PGC-LCs. During induction with BMP4, 17.52% (day 2), 26.57% (day 4), and 23.84% (day 6) of cells were EpCAM/CD49f-high (FIG. 7C). EpCAM-/CD49f-high cells showed significant upregulation of several PGC markers when compared to uninduced FTW-hiPSCs (day 0) (FIG. 7D). Moreover, IF analysis indicated that PGC markers: TFAP2C, PRDM1 and NANOS3 were also expressed at the protein level (FIG. 7E). Similar to mouse XPSCs, FGFR inhibition improved (albeit to a lesser extent than mouse) while WNT/LIF pathway inhibition reduced PGC-LC induction efficiency (FIG. 14E).

Taken together, these results indicate that, similar to mouse and horse XPSCs, FTW-hiPSCs also showed molecular and cellular features characteristic of formative pluripotency and are amenable to direct PGC-LC induction in vitro. Based on these and their chimeric competence in early pig embryos demonstrated previously, FTW-hiPSCs are hereafter referred to as human XPSCs (hXPSCs).

### Example 10. Cross-Species Transcriptome Comparison of XPSCs

XPSCs from multiple species generated and cultured in the same condition enabled examination of species-specific pluripotency features not interfered by different culture parameters. To this end, cross-species transcriptomic comparison of horse, mouse, and human XPSCs was performed. UHC analysis showed that horse XPSCs clustered closer with human than mouse XPSCs (FIG. 14F). The analysis identified 1,220, 1,269 and 1,540 genes specifically expressed in human, horse and mouse XPSCs, respectively (FIG. 14G). Top enriched species-specific GO terms include: 1) human: axonogenesis and neuron projection guidance; 2) horse: intracellular signal transduction and anterior/posterior pattern specification; 3) mouse: DNA methylation or demethylation and DNA modification (FIG. 14G). These results reveal species differences in pluripotency features conferred by the FTW culture and may help gain insights into evolutionary convergent and divergent processes underlying epiblast development across different species in vivo.

### Example 11. Materials and Methods

Mice

C57BL/6 and CD-1 (ICR) mice were purchased from Charles River, or Envigo (Harlen). 129S1/SvImJ and B6(Cg)-Tyrc-2J/J mice were purchased from the Jackson Laboratory. Mice were housed in 12-hr light/12-hr dark cycle. All procedures related to animals were performed in accordance with the ethical guidelines of the University of Texas Southwestern Medical Center. Animal protocol was reviewed and approved by the UT Southwestern Institutional Animal Care and Use Committee (IACUC) [Protocols #2018-102430].

Harvesting and Culture of Mouse Embryos.

C57BL/6 female mice (8-10 weeks old) were superovulated by intraperitoneal (IP) injection with 5 IU of PMSG (Prospec), followed by IP injection with 5 IU of hCG 48 h later. After mating with C57BL/6 male mice, embryos at 8-cell to morula stages were harvested at E2.75 [the presence of a virginal plug was defined as embryonic day 0.5 (E0.5)) in KSOM-Hepes (Wu et al., 2017) by flushing oviducts and uterine horns. Embryos were cultured in the mKSOMaa (Wu et al., 2017) overnight until blastocyst stage in a humidified atmosphere containing 5% (v/v) CO2 and 5% (v/v) 02 at 37° C. CD-1 females (8 weeks old or older) in natural estrous cycles were mated with CD-1 males. Blastocysts were harvested at E3.5 by flushing uterine horns.

Generation of Equine Somatic Cell Nuclear Transfer (SCNT) Embryos

All chemicals and reagents were purchased from Sigma Chemical Co. (St. Louis, MO), unless otherwise indicated.

Collection and in vitro maturation of equine oocytes: Equine ovaries from slaughterhouse (Mercedes, Buenos Aires) were transported to the laboratory within 3 h at 25° C. in sterile saline solution supplemented with 0.1 mg/mL streptomycin. For aspiration, follicles 2-10 mm in diameter were punctured using 19G needles connected to a vacuum pump. Follicular fluid containing cumulus-oocyte complexes (COCs) were collected in sterile 50 mL centrifuge tubes (Corning) with collection media [T199 with Hank's salts, 1 mg/ml of bovine serum albumin (BSA), 1 mg Heparin]. Only COCs with at least, three layers of cumulus cells were selected for in vitro maturation (IVM). Forty to forty-five COCs were cultured in 3 mL of maturation medium and incubated at 38.5° C. in 5% CO2 in air with maximum humidity for 25 h. The maturation medium consisted of M199 supplemented with 1× ITS, 0.2 mM sodium pyruvate, 100 μg/mL gentamicin, 10 ng/mL Long hIGF, 10 ng/mL mEGF, 0.1 IU recombinant human follicle-stimulating hormone (FSH) (Puregön, Organon) and 10% (v/v) fetal calf serum (FCS) (GIBCO).

Somatic cell nuclear transfer (SCNT): Primary fibroblasts (EF) were established from a skin biopsy of a 10 years old polo pony mare and cultured in DMEM high glucose (GIBCO) supplemented with 15% FCS at 38.5° C. in 5% CO2 and humidified air. All procedures were performed as described by Galli C. et al. (2006), with minor modifications. After h of maturation, loosely associated cumulus cells were removed by vortexing for 1-2 min in Hepes-buffered Synthetic Oviduct Fluid (H-SOF) (SOFaa supplemented with 20 mM HEPES) containing 1 mg/mL hyaluronidase and Trypsin 0.25% (v/v; GIBCO). Denuded oocytes were washed and returned to maturation medium. Enucleation process was initiated within 30 min of oocyte denudation under an inverted microscope (Nikon Eclipse Ti, Nikon) with micromanipulators (NT 88 V3, Nikon). Oocytes were incubated for 5-10 min in H-SOF supplemented with 1 mg/mL Hoechst 33342 and 5 μg/mL Cytochalasin B at 38° C., subsequently placed into manipulation drops (H-SOF supplemented with 1% FCS covered with mineral oil and μg/mL Cytochalasin B) and enucleated after a brief exposure to UV light (Nikon Filter Set 01, Nikon) to determine the location of DNA.

Passage 3-6 donor EF cells were collected from the culture plates by trypsinization using 0.05% (w/v) trypsin-EDTA, washed twice, and finally resuspended in H-SOF. Cells were picked up with the transfer needle and slipped into the perivitelline space of enucleated oocytes. Cell-cytoplast couplets were fused immediately after cell transfer using a 0.5-mm gap fusion chamber (BTX, San Diego, CA) overlaid with sorbitol fusion medium (0.25 M Mannitol, mM Calcium Chloride, 0.1 nM Magnesium Sulfate) with 30 psec of 2.7 kV/cm pulse (BTX Electrocell Manipulator ECM 2001, Harvard Apparatus, MA). The post-fusion culture medium consisted of SOF supplemented with 10% (v/v) FCS. After 1.5 h, the fused couplets were activated by exposure to 8.7 μM ionomycin in H-SOF medium for 4 min, then rinsed three times in H-SOF and allocated to a 4-h culture in 1 mM 6-DMAP and 5 ug/mL Cycloheximide at 38.5° C. in 5% CO2 in air with maximum humidity. After this treatment, presumptive embryos were rinsed in H-SOF and cultured for 7 days in Global medium (Global) with 10% FCS (Hyclone defined) and 8 mg/mL BSA (Probumin, Millipore).

Sheep and Goat In Vitro Embryo Production

COCs recovery and IVM: Sheep and goat ovaries were collected from Superior Farm abattoir in Dixon, California. Ovaries were transported to the laboratory in warm (˜37° C.) saline supplemented with 1× Penicillin-Streptomycin solution. Upon arrival to the laboratory, ovaries were washed in warm tap water and kept in saline at 37° C. during processing. Sheep and goat COCs were aspirated from 2-6 mm antral follicles using a 21 G butterfly needle connected to a vacuum pump. The vacuum pressure was adjusted to a flow rate of 10-12 mL/min. Oocytes with several layers of cumulus cells were selected, washed and matured in vitro in TCM199 supplemented with 10% (v/v) Ovine Estrus Serum (OES), ovine FSH (50 ng/mL; National Hormone & Peptide Program, UCLA), bovine LH (3 mg/mL; Sioux Biochemical), and cysteamine (0.1 mM) for 22 h in 5% CO2 with humidified atmosphere at 38.5° C.

In vitro fertilization: Expanded COCs were washed twice and placed in fertilization medium consisting of SOF supplemented with 2% OES, 10 μg/mL heparin, and 10 μg/mL hypotaurine. Motile sperm from fresh ejaculate were selected by swim-up method, and their concentration was adjusted to 2×106 sperm/mL, then co-incubated with the expanded COCs for 16 h in 5% CO2 with humidified atmosphere at 38.5° C.

In vitro culture: Presumptive embryos were denuded from the surrounding cumulus cells by vortexing in SOF-Hepes medium containing hyaluronidase (1 mg/mL) for 3 min and washed with SOF-Hepes, then cultured in groups of 25 in 50 μL drops of BO-IVC (IVF Bioscience) under oil at 38.5° C., 5% (v/v) CO2 and 5% (v/v) 02 for 4 days.

Pig Parthenogenetic Embryo Production

Oocyte Collection and IVM: Oocytes were aspirated from antral follicles (2-4 diameters) of ovaries from prepubertal gilts collected at a local slaughterhouse (Olson Meat Company, Orland, CA). COCs were washed in TCM-199 (GIBCO) containing 0.1% (w/v) polyvinyl alcohol (PVA), and incubated at 38° C. and 5% CO2 for 48 h in TCM-199 containing PVA, 3.05 mM D-glucose, 0.91 mM sodium pyruvate, 0.5° FSH, 0.5 bLH, ng/mL EGF, 10 μg/mL gentamicin (GIBCO) and 10% (v/v) porcine follicular fluid.

Parthenogenetic Activation: After IVM, maturated oocytes were stripped of their cumulus cells by incubation in 1 mg/mL hyaluronidase and gentle pipetting. Denuded oocytes were washed with MEM containing 25 mM HEPES (GIBCO) and electrically activated with two pulses of 120 V/mm for 40 delivered by a BTX Electro Cell Manipulator 2001 (BTX, San Diego, CA) in a 0.5 mm chamber containing 0.3 M mannitol, 0.05 mM CaCl2, 0.1 mM MgSO4 and 0.1% (w/v) BSA. After washing with PZM-5 (Yoshioka et al., 2012), the oocytes were incubated in the presence of 5 μg/mL cytochalasin B in PZM-5 for 3 h to prevent second polar body extrusion and thus generate diploid parthenogenetic embryos.

Embryo Culture: After activation, pig zygotes were cultured in the 500 μL of PZM-5 (Yoshioka et al., 2012) containing 0.3% BSA for 3-days at 38.5° C. in a humidified atmosphere of 5% CO2, 5% 02, and 90% (v/v) N2.

Derivation and Culture of Mouse ESCs, EpiSCs and FTW-ESCs Lines

Mouse ESCs and EpiSCs derivation: Embryo manipulations were performed under a dissection microscope (Nikon SMZ800N). In brief, zona pellucidae (ZP) were removed from E3.5 blastocysts by brief treatment with acidic Tyrode's solution (Millipore MR-004-D). After removing zona pellucidae, embryos were plated on MEFs in NB2iL medium for mESCs derivation (chemically defined N2B27 basal medium (De Los Angeles et al., 2019) supplemented with human leukemia inhibitory factor (LIF) (10 ng/mL, Peprotech), CHIR99021 (3 Selleckchem) and PD0325901 (1 μM, Selleckchem)) or in NBFR medium for EpiSCs derivation (Wu et al., 2015) (N2B27 basal medium supplemented FGF2 (20 ng/mL, Peprotech) and IWR1 (2.5 μM)). After 4-6 days in culture, ICM outgrowths were passaged using TrypLE and re-seeded onto newly prepared MEFs. EpiSCs clones were manually picked for further cultivation to avoid contamination of trophoblasts.

Mouse FTW-ESCs derivation from blastocyst. ZP-removed mouse blastocyst stage embryos [C57BL/6, CD-1 (ICR), 129S1/SvImJ and B6(Cg)-Tyrc-2J/J] were obtained as above described. Then embryos were plated on MEFs in FTW derive medium [N2B27 basal medium supplemented with FGF2 (10 ng/mL, Peprotech), Activin-A (10 ng/mL, Peprotech), CHIR99021 (3 Selleckchem) and PD0325901 (0.5 Selleckchem)]. After 4-6 days in culture, ICM outgrowths were passaged using TrypLE and re-seeded onto newly prepared MEFs. Once the homogenous colonies can be observed (2-3 days after re-seeding), change the medium into normal FTW medium [N2B27 basal medium supplemented with FGF2 (10 ng/mL, Peprotech), Activin-A (10 ng/mL, Peprotech) and CHIR99021 (3 Selleckchem)]. Optional: FTW-ESCs clones can be manually picked for further cultivation to avoid contamination of trophoblasts.

Culture of Mouse ESCs, FTW-ESCs and mEpiSCs: Established mouse ESC, FTW-ESC and mEpiSC lines were cultured on MEFs coated plates and passaged every 3-4 days at a split ratio of 1:20 (mESCs) or 1:20 (FTW-mESCs) or 1:50 (mEpiSCs).

Derivation of FTW-eqESCs

Vitrified horse blastocysts produced by intracytoplasmic sperm injection (ICSI) and somatic cell nuclear transfer (SCNT) were sent in dry shippers and placed in liquid nitrogen upon arrival. Vitrified equine morula stage embryos were warmed as described previously (Y.-H. Choi and Hinrichs, 2017). Briefly, embryos were warmed in Holding medium [Hanks solution (GIBCO #12350-039) supplemented with 20% FBS] with washes at decreasing sucrose concentrations of 0.25 M, 0.15 M and intervals of 1 and 5 min respectively, followed by a 5 min incubation in holding medium. ZP was removed with Tyrode's solution for 30 sec under an inverted microscope with warming stage. Embryos were cultured in FTW medium supplemented with 10% serum on MEF feeders in a 37° C. humidified incubator at atmosphere of 5% CO2. Serum was withdrawn after attachment of the embryos. Expanded blastocysts that did not attached to the MEF feeder layer were either micro-dissected in Ca2+, Mg2+ free PBS with a microblade (Shearer precision products) under inverted scope with micromanipulation system or dissociated under mild trypsinization under an inverted scope. Well defined FTW-eqESCs colonies were reseeded into new MEFs plates.

Generation of FTW-eqiPSCs and FTW-hiPSCs with episomal vectors

Horse embryonic fibroblasts were isolated from a day 23 embryo collected by trans-cervical uterine flushing of a warmblood mare. Equine fetal fibroblast (EFFs) cells and Human foreskin fibroblasts (HFFs) were cultured in fibroblast growth medium (DMEM supplemented with 10% FBS). Episomal plasmids: pCXLE-hOCT3/4-shp53 (Addgene plasmid #27077), pCXLE-hSK (Addgene plasmid #27078), pCXLE-hUL (Addgene plasmid #27080) and pCXLE-EGFP (Addgene plasmid #27082) were used for reprogramming (Okita et al., 2011). 1.5 ng of each episomal plasmid (total 6 ng) were delivered into 2×106 EFFs or HFFs with nucleofector (Lonza) according to the manufacturer's instructions. NHDF program (Pulse code DT130) and Primary Cell P2 solution (for EFFs) and P3 solution (for HFFs) (Lonza) were used for nucleofection. The cells were trypsinized 5 days after nucleofection, and reseeded onto 100 mm dishes coated with MEFs. The culture medium was replaced the next day with N2B27 medium containing 10 ng/mL bFGF (Peprotech), 10 ng/mL Activin A (Peprotech), and 3 μM ChIR99021 (Selleckchem). Individual colonies were picked between 14-24 days after reseeding.

FTW-eqPSCs and FTW-hiPSCs Cell Culture

FTW-eqPSCs were maintained in FTW medium (N2B27 medium supplied with 10 ng/mL bFGF, 10 ng/mL Activin A, and 3 μM CHIR99021 or 50 ng/ml WNT3a) on MEFs or Matrigel (BD Biosciences) coated dish. The cells were cultured at 37° C. and 5% CO2, and medium was changed daily. For passaging, FTW-eqPSCs were dissociated into single cells using TrypLE Express (GIBCO) and passaged at 1:20 every 3-4 days.
Alkaline Phosphatase (AP) Staining

Cells were fixed in 4% (w/v) PFA for 15 min at room temperature. AP substrate solution (System Biosciences) was prepared per manufacturer's instructions. The cells were incubated with AP substrate at room temperature for 15 to 20 min in the dark.
Karyotype Analysis

Cells were incubated in KaryoMAX Solution (GIBCO) for 1 h. Then the cells were collected and resuspended in 75 μM KCl solution. After incubation for 30 min at 37° C., 0.5 mL fixative solution (3:1, v/v, methanol:acetic acid) was added to the KCl solution and centrifuged at 300 g for 10 min. Then the supernatant was removed, and 5 mL of cold fixative solution was added followed by incubation on ice for 30 min. The above steps were repeated and the final cell suspension was dropped onto glass slides, and stained with 300 nM 4′-6-diamidino-2-phenylindole (DAPI) solution.

Episomal Vectors Copy-Number Detection

The copy-number of episomal vectors were detected as previous reported (Okita et al., 2011). Briefly, FTW-eqiPSCs cultured on feeder-free conditions were dissociated, or FTW-hiPSCs cultured on feeder were purified by MACS Feeder Removal MicroBeads kit (Miltenyi Biotec) and genomic DNA was extracted for quantitative RT-PCR analysis. Primers for the episomal plasmid backbone sequence (EBNA-1) were used to estimate the total copy-number of all for transfected episomal vectors, and eq-GAPDH was used to estimate the cell number. The copy-number in each sample was estimated from the observed Ct values of EBNA-1. The cell number was estimated from the observed eq-GAPDH Ct values+1, as each cell has two GAPDH alleles.

Clonal Efficiency Assay

Dissociated cells were gently passed through a 40-μm cell strainer and plated in a 6-well plate at clonal density (˜200 cells/well). Cells cultured in FTW medium with and without 10 mM Y-27632 treatment. Colonies (>20 cells) were counted after 10 days. The clonal efficiency was calculated by dividing the number of colonies by the total cell numbers plated.

RT-PCR and qRT-PCR Analysis

Total RNA was isolated using the RNeasy Mini Kit (QIAGEN) following the manufacturer's instructions. Genomic DNA was degraded by RNase-Free DNase Set (QIAGEN). RNA concentrations were measured on a spectrophotometer (DS-11+, DeNovix). cDNA of total RNA was synthesized with iScript Reverse Transcription Supermix kit (BIO-RAD), and amplified with PrimeSTAR GXL DNA Polymerase (TaKaRa) or with SYBR Green PCR Master Mix (Thermo Fisher Scientific) on a Touch Thermal Cycler Real-Time PCR system (C1000, BIO-RAD). GAPDH expression level was used as an internal normalization control.

RNA FISH

Mouse Xist probes with Quasar 570 dye were purchased from Biosearch Technologies (SMF-3011-1). FISH hybridization was performed following manufacture's protocol.

Cell Population Doubling Time

The cell population doubling time was calculated using a doubling time online calculator (doubling-time.com/compute.php?lang=en).

Embryoid Bodies (EBs) Formation

Embryoid bodies (EBs) were generated by using AggreWell 400 plates (Stem Cell Technologies) per manufacturer's instructions. After 24 h, formed EBs were harvested and cultured in an ultra-low attachment 24-well plate (Corning). After 1 week, EBs were transferred to Matrigel-coated dishes and cultured for additional 4-6 weeks in DMEM medium supplemented with 10% FBS.

Teratomas Formation

A total of 5×106 cells was suspended in 50 μL of DMEM-Matrigel solution and injected subcutaneously into 10-week-old immunodeficiency NOD-SCID mice. After 6-8 weeks, teratomas were dissected and fixed with Bouin's solution. Paraffin-embedded teratomas were sliced and stained with hematoxylin and eosin.

Directed Differentiation of FTW-eqPSCs into Cardiomyocytes

FTW-eqiPSCs were differentiated into cardiomyocytes by using WiGi protocol that has been previously reported (Lian et al., 2012). Briefly, FTW medium was replaced with RPMI 1640 medium containing B27 minus insulin supplement (RPMI/B27-insulin, GIBCO) and 12 μM of CHIR99021 (Selleckchem). After 1 day, the medium was replaced with fresh RPMI/B27-insulin medium. On day 3, the culture medium was replaced with RPMI/B27-insulin medium supplemented with 5 μM IWP2 (Peprotech). On day 5, the medium was replaced with fresh RPMI/B27-insulin medium. Thereafter, the culture medium was replaced with RPMI 1640/B27 every 3 days.

Directed Differentiation of FTW-eqPSCs into Neurons

To differentiate FTW-eqiPSCs into neurons, we used a published protocol (Shi et al., 2012). Briefly, FTW-eqiPSCs were dissociated into single cells using TrypLE and plated in (w/v) gelatin coated plate for 30 min at 37° C. to remove MEFs. The nonadherent FTW-eqiPSCs were plated in Matrigel-coated plate at a density of 10,000-25,000 cells/cm2 in FTW medium. The cells were allowed to expand in FTW Medium for 2-3 days until they were 80— 90% confluent. At which point, the medium was changed to neural induction medium, which contains 1:1 mixture of N2B27 media with 10 μM SB431542 (Tocris) and 500 ng/ml of Noggin (R&D). After 8-12 days differentiation, passage the neuroepithelial sheet use Dispase (Stemcell) by 1:3 ratio into Poly-L-ornithine and Laminin coated dishes in N2B27 medium (without SB431542 and Noggin). On day 12-16, 20 ng/mL of FGF2 was added to the medium for 2-4 days, and then continue to culture up to 30 days in N2B27 medium.

Immunostaining of Mouse, Horse, Sheep and Goat Blastocysts

48 h after FTW-eqiPSCs injection blastocysts at were washed three times in PBS containing 0.1% (w/v) PVA (PVA-PBS) and treated with Tyrode's solution to remove ZP. After washing three times in PVA-PBS, they were fixed in 4% PFA containing 0.1% PVA at room temperature for 30 min. After three-time washes, blastocysts were permeabilized with 1% (v/v) Triton X-100 (Fisher BioReagents) in PVA-PBS for 30 min and washed three times in PVA-PBS containing 0.1% Triton X-100 (washing buffer). After blocking in 10% (v/v) normal donkey serum and 0.3% Triton X-100 in PVA-PBS for 1 h, blastocysts were incubated with anti-OCT3/4 (1:50 diluted, sc-365823, Santa Cruz Biotechnology) (for mouse blastocysts), or anti-SOX2 (1:50 diluted, sc-365823, Santa Cruz Biotechnology) (for horse, sheep and goat blastocysts) at 4° C. overnight. After rinse in washing buffer, embryos were incubated in Alexa Fluor 488 anti-mouse IgG (1:200 diluted, A21202, Invitrogen) at room temperature for 1 h. After washing, blastocysts were counterstained with 20 ng/mL of DAPI for 20 min, mounted on the glass slide with small volume of PVA-PBS, covered by coverslip, and imaged using a fluorescence (Echo Laboratories, CA) or a confocal microscope (LSM700, Carl Zeiss).

Immunofluorescence Staining

Cells were fixed in 4% PFA buffer for 15 min, and permeabilized with 0.5% Triton X100 in PBS for 5 min at room temperature. Cells were blocked with blocking buffer [5% (v/v) BSA; and 0.1% (v/v) Tween 20 in PBS] for 1 h and incubated with the following primary antibodies diluted in blocking buffer at room temperature for 2 h or at 4° C. overnight: anti-OCT4 (1:50 diluted, sc-5279, Santa Cruz Biotechnology), anti-SOX2 (1:50 diluted, sc-365823, Santa Cruz Biotechnology), anti-KLF4 (1:500 diluted, AF3158, R&D), anti-SSEA1 (1:100 diluted, sc-21702, Santa Cruz Biotechnology), anti-ZO1 (1:500 diluted, 33-9100, Life Technology), anti-human β-tubulin III (1:50 diluted, sc-5274, Santa Cruz Biotechnology), anti-Cardiac troponin T (1:400 diluted, ab47003, Abcam), and anti-Alpha-fetoprotein (10 μg/mL, MAB1368, R&D Systems), Lys9 (1:100 diluted, 9753, Cell Signaling), Lys27 (1:100 diluted, 9733, Cell Signaling). Then, the cells were incubated with secondary antibodies (Alexa Fluor 488 or 594 donkey anti-rabbit or mouse IgG (1:300 diluted, Invitrogen)) in blocking buffer at room temperature for 1 h. Finally, cells were counterstained with 300 nM DAPI solution at room temperature for 20 min. After each step, samples were washed with PBS three times.

Western Blot

Cultured cells were detached as above culture conditions, cell number were counted. Cell were lysed with Laemmli Sample Buffer (Bio-Rad). Cell lysate were separated by 10% Bis-Tris Protein Gel or NuPAGE 4%-12% Bis-Tris Protein Gel (Invitrogen) in running buffer according to different protein size. Protein were electrophoretically transferred into 0.2 μm PVDF membranes (1620177, BIO-RAD) with transfer buffer. Blots were blocked at room temperature for 1 hour in TBS-T+5% fat-free milk (Kroger) and probed overnight at 4° C. with primary antibody (1:1000) in 1% TBS-T BSA. Primary antibody information is as follows: GAPDH (1:1000 diluted, M4AB37, Millipore), VINCULIN (1:1000 diluted, AB129002, Abcam), OCT4 (1:1000 diluted, sc-5279, Santa Cruz Biotechnology), NANOG (1:1000 diluted, AB5731, Millipore), OTX2 (1:1000 diluted, AF1979, R&D SYSTERMS), P-SMAD (1:1000 diluted, 138D4, Cell Signaling), p44/42 MAPK (1:1000 diluted, 137F5, Cell Signaling), P-p44/42 MAPK (1:1000 diluted, 9101, Cell Signaling), After washing three times in TBS-T, signals were detected by using secondary ELC Anti-mouse IgG (1:5000 diluted, NXA931V, GE Healthcare LifeScience) or ELC Anti-rabbit IgG (1:5000 diluted, NA934V, GE Healthcare LifeScience) antibodies conjugated to horseradish peroxidase. Secondary antibodies were incubated for 1 hour at room temperature. After three times washes, a piece ECL 2 western blotting substrate (80196, Invitrogen) was used to develop the films and blue X-Ray films (F-BX57, Phenix) were used to record the signals. Signal were scanned by Epson perfection V700 photo, and analyzed by Epson scan software.

Luciferase Assays

Cells (2E5 per well) were transfected with 2 ug TOPFlash or FOPFlash luciferase plasmids in 24-well plates. After 36 h, cells were lysed and analysed using the dual luciferase kit (Promega) according to the manufacturer's protocol.

Flow Cytometry

The cells were dissociated with TrypLE Express at 37° C. for 5 min. Then, the cells were fixed in 4% paraformaldehyde (PFA) at room temperature for 30 min and permeabilized with 0.5% v/v triton X-100 at room temperature for 30 min. The cells were then incubated in the primary antibody solution for 30 min and then the secondary antibody solution for 30 min at room temperature. Samples stained with only secondary antibodies were used as the negative control. Finally, the stained cells were washed with PBS containing 2% FBS twice, and the cell suspensions were applied to flow cytometry (FACScalibur system, BD).

Lumen Formation Assay

Cells were induced to polarize and form lumens using the 3D on top protocol as previously described (Lee et al., 2007). In brief, au-Slide-8-well ibiTreat plate (IB-80826, Ibidi) was coated with Matrigel (BD Biosciences) for 1 hour at 37° C. Then pellets containing 10,000 cells were resuspended in 50 μL of ice cold Matrigel (BD Biosciences). The solution was placed as a drop in a well of aμ-Slide 8-well ibiTreat plate and incubated at 37° C. for 5 min. Then 300 μL of culture medium was added to the wells.

Primordial Germ Cell Like Cell (PGC-LC) Induction

PGC induction from mESCs was performed following a published protocol (Hayashi and Saitou, 2013). For PGC-LCs induction, 1.5×103 cells (mouse FTW-PSCs or EpiLCs) or 3.0×103 cells (equine FTW-PSCs) were plated into each well of low-cell binding U-bottom 96-well plate (NUNC) in PGC-LC induction medium: GK15 medium containing GMEM, 15% (v/v) KnockOut serum replacement, 0.1 mM sodium pyruvate, 0.1 mM NEAA, 0.1 mM b-mercaptoethanol, 2 mM Glutamax and 100 U/mL penicillin, supplemented with 500 ng/mL (mouse FTW-PSCs and EpiLCs) or 200 ng/mL (FTW-eqPSCs) BMP4 (GIBCO), LIF (1000 U/mL; Peprotech), SCF (100 ng/mL; R&D) and EGF (50 ng/mL; Peprotech). In some experiment, the inhibitor was added including PD173017 (100 ng/mL; Selleckchem), SB431542 (10 μM; Tocris), IWR1 (2.5 μM; Sigma), or Pyridine-6 (1 μM; Sigma).

Blastocyst Injection of FTW-PSCs

FTW-PSCs injection into mouse blastocysts were performed as described previously (Wu et al., 2017) with slight modification. Briefly, single cell suspensions of FTW-PSCs were added to a 40 μL droplet of KSOM-Hepes containing the blastocysts and placed on an inverted microscope (Leica) fitted with micromanipulators (Narishige). Individual cells were collected into a micropipette with 15-20 μm internal diameters (ID), and Piezo Micro Manipulator (Prime Tech) was used to create a hole in the zona pellucida and trophectoderm layer of mouse blastocysts. Ten (FTW-mESCs) or fifteen (FTW-eqiPSCs) or twelve (FTW-eqESCs) cells were introduced into the blastocoel. After microinjection, the blastocysts were cultured in mKSOMaa. Fluorescence signals were observed using an inverted fluorescence microscope (Nikon) at 24 and 48 h post-injection.

For mouse embryo transfer, 8-12 weeks old ICR female mice were used as surrogates and were mated with vasectomized ICR male mice to induce pseudopregnancy. Ketamine (30 mg/ml) and Buprenorphine (1 mg/ml) were used in surgery for maintaining anesthesia and relieving pain. Injected embryos were transferred to the surrogate uterine at E2.5. 14-30 blastocysts were transferred and performed within 20-30 min per surrogate.

For injection of FTW-eqiPSCs into equine blastocysts, briefly, single cell suspensions of FTW-eqiPSCs were added to a 40 μL droplet of KSOM-Hepes containing the blastocysts. Individual cells were collected into a 20 μm ID of micropipette. A laser system (Saturn Active, Research Instruments) was used to create a whole in the zona pellucida and trophectoderm layer. Ten cells were introduced into the blastocoel. After microinjection, the blastocysts were cultured on MEF in FTW media for 48 h. Fluorescent signals were observed and imaged using an inverted fluorescence microscope at 24 and 48 h post-injection.

For sheep, goat and pig morula injections, embryos with more than eight blastomeres and before compaction were selected on days 3-4 for pig and day 4 for sheep and goat embryos. Single cell suspensions were added to a 50 μL drop of cell culture medium containing the embryos to be injected and placed on an inverted microscope fitted with micromanipulators. Individual cells were collected into micropipette of 20-30 μm ID. Then, a laser system was used to create a whole in the zona pellucida of the embryo. Then, 10 cells deposited between blastomeres. Following cell injection, embryos were cultured in mixed medium (1:1) of each cell culture medium and embryo culture medium. At the end of the culture period, hKO signals was observed using an inverted fluorescence microscope (Nikon) and then embryos were fixed for immunostaining.

Immunostaining and Imaging of Chimeric Embryos

At E13.5 (for FTW-mESCs) or E7.75/E9.5 (for FTW-eqESCs), surrogates were euthanized and embryos were isolated. Embryos were immersed in 4% paraformaldehyde and incubated at 4° C. for 1 hour (small-sized embryos) or overnight (normal-sized embryos). After overnight cryoprotection in 30% sucrose solution (Fisher), the embryos were embedded in Polyfreeze Tissue freezing medium (Polyscience, Inc) and frozen in dry ice. Sections (10-12 mm thick for small-sized embryos and 20 mm for normal-sized embryos) of the different embryos were cut on a Leica cryostat (Leica CM1950). For immunostaining, we used standard staining procedures and 10 mM citrate buffer (0.05% tween20 based) for antigen retrieval. The primary antibodies used as following: chicken anti-GFP (AVES Lab, 1:300), Mouse anti-TuJ1 (Santa Cruz, 1:50), Mouse anti-Nestin (Santa Cruz, 1:50), Rabbit anti-T (Abcam, 1:1000), Rabbit anti-cTNT (Abcam, 1:100), Mouse anti-SMA (Abcam, 1:300), Mouse anti-50X17 (Santa Cruz, 1:50), Rabbit anti-FOXA2 (Cell Signaling, 1:200), Mouse anti-AFP (R&D, 1:100), Rabbit anti-PAX6 (Santa Cruz, 1:100). Imaging of chimeric embryos was performed with a Zeiss Axio Zoom.V16 fluorescence stereo zoom microscope equipped with a Plan-Neofluar Z 1.0x/0.25 (FWD 56 mm) objective and Axiocam 503 monochromatic camera. All pictures were taken using same aperture, exposure and gain acquisition settings. Fluorescent EGFP images were pseudo colored using the ZEN PRO 2012 (blue edition, ZEISS) software. 2.3.

Post-Implantation Mouse Embryo In Vitro Culture

The mouse embryos were in vitro cultured into post-implantation stage by using a published protocol (Bedzhov et al., 2014). Briefly, the cells-injected mouse embryos were plated on au-Slide-8-well ibiTreat plate (IB-80826, Ibidi) in IVC1 medium: advanced DMEM/F12 supplemented with 20% (v/v) heat-inactivated FCS, 2 mM Glutamax (GIBCO), 1× ITS-X (Thermo Fisher), 8 nM β-estradiol, 200 ng/ml progesterone and 25 μM N-acetyl-1-cysteine. Once the embryos were attached, change the medium into IVC2 medium: DMEM/F12 supplemented with 30% (v/v) KSR, 2 mM Glutamax, 1× ITS-X, 8 nM β-estradiol, 200 ng/ml progesterone and 25 μM N-acetyl-1-cysteine. After that, change IVC2 medium every two days.

RNA-Sequencing

RNA extraction was performed using a RNeasy Mini Kit (QIAGEN) using DNase treatment (QIAGEN). RNA was analyzed using a 2100 Bioanalyzer (Aglient Technologies). (Transcripts per Kilobase Million). RNA-seq reads were mapped to the mouse genome (GRCm38.p6) and horse genome (EquCab3.0) using HISAT2 (version 2.1.0)(Kim et al., 2015) with parameters “-k 1 --no-unal--dta--rna-strandness R”. The gene expression levels were then calculated using StringTie (v1.3.3b)(Pertea et al., 2015) with parameters “--rf -t -e -B -A”. A 2-fold variance in expression levels, a P value less than 0.05 and an adjusted P value less than 0.1 were used as cutoffs to define differentially expressed genes. The P value and adjusted P value were calculated using DESeq2(Love et al., 2014). GO analysis preformed on Metascape (metascape.org).

ChIP-Sequencing

The cells were dissociated with TrypLE Express at 37° C. for 5 min, then rinsed with ice-cold PBS. Cells were cross-linked in fresh 1% PFA at room temperature for 8 minutes. Cross-linking was quenched by glycine (final concentration 0.125M), and samples were placed on a rotator at room temperature for an additional 10 min. Collect cells by centrifugation at 2000 g for min at 4° C. Remove the supernatant and rinse cells with ice-cold PBS. Dislodge the pellet by flicking the tube with finger. Then harvest the cells by centrifugation at 2000 g for 10 min at 4° C. and discard the supernatant. Flash freeze the cell pellet in liquid nitrogen and stored at −80° C. ChiP were performed by using a Diagenode iDeal ChIP-seq kit for Histones (C01010059, Diagenode). Solubilized chromatin was immunoprecipitated with antibody against H3K4me3 (A1052D, Diagenode) and H3K27me3 (A0821D, Diagenode). Library were prepared by using a NEBNext Ultra II DNA Library Prep Kit (E7645L, NEB). ChIP-seq reads were mapped to the mouse genome (GRCm38.p6) and horse genome (EquCab3.0) using bowtie2 (version 2.3.4.3)(Ben Langmead and Salzberg, 2012) with parameters “-k 1 -q”. Potential PCR duplicates were then removed using Samtools rmdup (version 1.5)(H. Li et al., 2009). Peak calling was performed using MACS2 (version 2.1.1.20160309)(Y. Zhang et al., 2008) with parameters “-t -c -g -B -p 0.001”. Peaks were annotated using ChlPseeker (v1.18.0)(Yu et al., 2015).

Visualization of RNA-Seq and ChIP-Seq Data

The transcriptome and ChIP-seq data sets were visualized using Integrative Genomics Viewer (IGV, version 2.3.88)(Robinson et al., 2011).

Statistical Analysis

All quantitative data are presented as the mean±SD. Experiments were repeated at least three times (repeat number was indicated as “n” in figure legends). Statistical analysis was performed by using a multiple t test analysis with Prism (GraphPad Software). P values less than 0.05 were considered to be statistically significant.

### Example 12. Successful Derivation of Human, Rhino and Pig iPSCs or ESC

Further optimization on the FTW-PSC culture condition for human, rhino and pig PSCs was conducted.

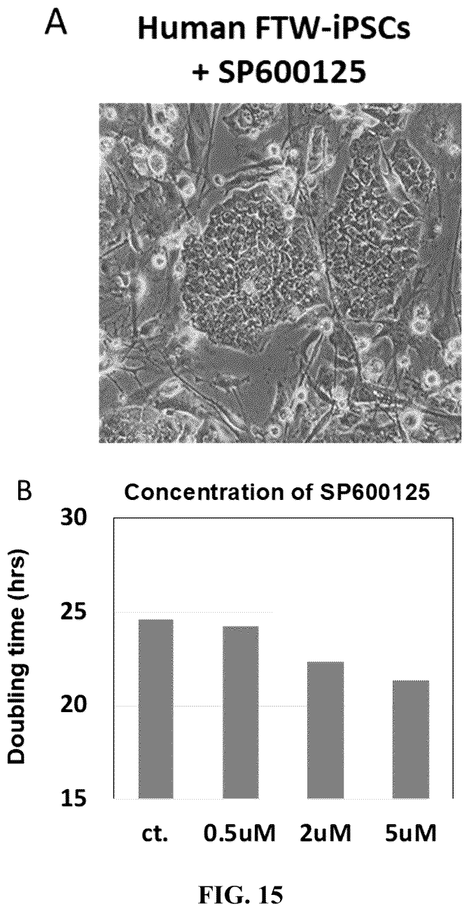

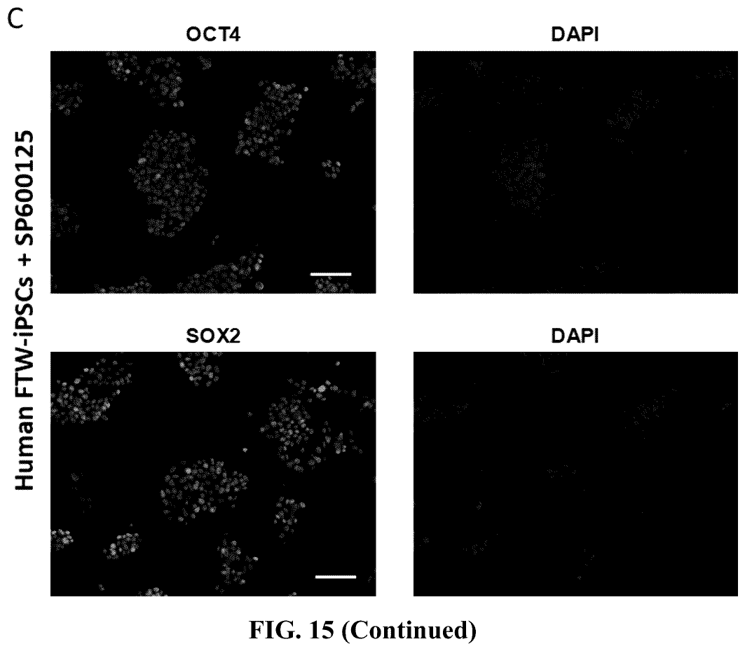

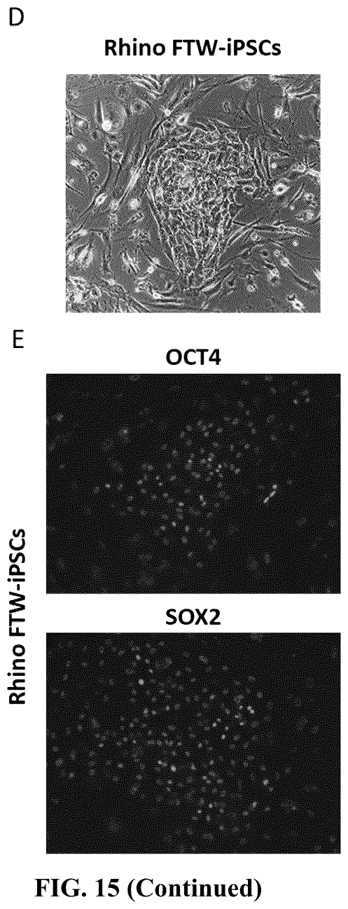

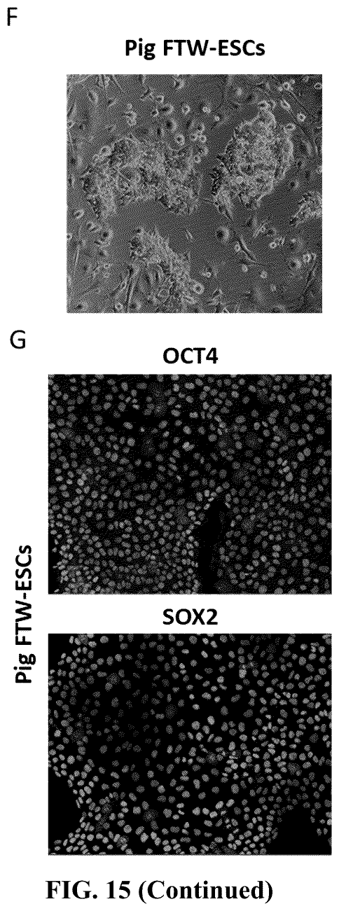

For human FTW-PSC cells, following derivation of the pluripotent cells according to methods described in Examples 8 and 11, human FTW-PSC cells were cultured in the presence or absence of a INK inhibitor (SP600125, 0.5 μM, 2 μM, or 5 μM). FIG. 15A shows representative brightfield images of cells cultured in the presence of the INK inhibitor and FIG. shows that including the INK inhibitor greatly increased the growth (e.g., decreased doubling time) of human FTW-PSCs in a dosage dependent manner. The human FTW-PSCs also expressed the same pluripotency markers (e.g., OCT4 and SOX2) as expected as shown in FIG. 15C.

To test whether the FTW-PSC protocol could work on rare species, FTW-iPSCs were derived from rhino terminal cells (fibroblasts) using the same protocol for human FTW-iPSCs (see Examples 8 and 11). These cells grew effectively (FIG. 15D) and expressed markers of pluripotency (OCT4 and SOX2), (FIG. 15E).

Finally, pig FTW-PSCs were derived using the FTW-PSC protocol as previously described. Briefly, Leukemia Inhibitory Factor (LIF) was added to both the differentiation medium and the culturing medium. In this experiment it was shown that the FTW condition plus Leukemia Inhibitor Factor (LiF) enabled the derivation of pig ESCs from pig blastocysts (FIG. 15F-15G).

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same FAShion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same FAShion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted elements.

1. A method for producing formative embryonic stem cells (ESCs), the method comprising:
(i) obtaining a population of reproductive cells, and
(ii) culturing the population of reproductive cells in a medium that comprises a fibroblast growth factor (FGF) activator, a transforming growth factor beta (TGF-β) activator, and a WNT activator,
thereby producing formative ESCs.

2. The method of claim 1, wherein the population of reproductive cells comprises blastocysts.

3. The method of claim 1 or claim 2, wherein the population of reproductive cells are obtained from a subject.

4. The method of claim 3, wherein the subject is selected from the group consisting of a human, a rodent, and an ungulate.

5. The method of any one of claims 1-4, wherein the FGF activator is selected from the group consisting of a protein, a nucleic acid, a small molecule, and a combination thereof.

6. The method of claim 5, wherein the FGF activator is FGF protein.

7. The method of any one of claim 1-6, wherein the TGF-β activator is selected from the group consisting of a protein, a nucleic acid, a small molecule, and a combination thereof.

8. The method of claim 7, wherein the TGF-β activator is TGF-β protein.

9. The method of claim 7, wherein the TGF-β activator is Activin A protein.

10. The method of any one of claims 1-9, wherein the WNT activator is selected from the group consisting of a protein, a nucleic acid, a small molecule, and a combination thereof.

11. The method of claim 10, wherein the WNT activator is WNT protein.

12. The method of any one of claims 1-11, wherein the WNT activator is a GSK3 inhibitor and/or a MEK inhibitor.

13. The method of claim 12, wherein the GSK3 inhibitor is CHIR99021.

14. The method of any one of claims 1-13, wherein step (ii) is performed for 1 to 8 days.

15. The method of any one of claims 1-14, wherein, prior to step (ii), the population of reproductive cells is cultured in a medium that comprises at least one additional factor.

16. The method of any one of claims 1-15, wherein the medium in step (ii) further comprises at least one additional factor.

17. The method of claim 16, wherein the at least one additional factor is a MEK inhibitor, fetal bovine serum (FBS), leukemia inhibitory factor (LIF), a INK inhibitor, or a combination thereof.

18. The method of claim 17, wherein the MEK inhibitor is PD0325901.

19. The method of claim 17, wherein the INK inhibitor is SP600125.

20. The method of any one of claims 1-19, wherein the formative ESCs produced in step (ii) are cultured in the presence of at least one differentiation factor, wherein the at least one differentiation factor is selected from the group consisting of a growth factor, a WNT inhibitor, a INK inhibitor, and a TGF-β inhibitor.

21. The method of claim 20, wherein the growth factor is fibroblast growth factor (FGF), Noggin, or a combination thereof.

22. The method of claim 20 or claim 21, wherein the WNT inhibitor is IWP2.

23. The method of any one of claims 20-22, wherein the TGF-β inhibitor is SB431542.

24. The method of any one of claims 20-23, wherein the INK inhibitor is SP600125.

25. A method for producing formative induced pluripotent stem cells (iPSCs), the method comprising:
(i) obtaining a population of somatic cells,
(ii) culturing the population of somatic cells in a first medium that comprises a reprogramming agent that modulates expression of at least one reprogramming gene selected from the group consisting of OCT3/4, p53, SOX2, KLF4, L-MYC, and LIN28, and
(iii) culturing the population of somatic cells in a second medium that comprises a fibroblast growth factor (FGF) activator, a transforming growth factor beta (TGF-β) activator, and a WNT activator,
thereby producing formative iPSCs.

26. The method of claim 25, wherein the population of somatic cells comprises embryonic fibroblasts.

27. The method of claim 25 or claim 26, wherein the population of somatic cells are obtained from a subject.

28. The method of claim 25, wherein the subject is selected from the group consisting of a primate, a rodent, and an ungulate.

29. The method of any one of claims 25-28, wherein the reprogramming agent comprises a nucleic acid.

30. The method of claim 29, wherein the nucleic acid encodes the reprogramming gene.

31. The method of claim 29, wherein the nucleic acid encodes an interfering RNA that targets the reprogramming gene.

32. The method of any one of claims 29-31, wherein the nucleic acid is comprised in a vector.

33. The method of claim 32, wherein the vector is an episomal vector.

34. The method of any one of claims 25-33, wherein the FGF activator is selected from the group consisting of a protein, a nucleic acid, a small molecule, and a combination thereof.

35. The method of claim 34, wherein the FGF activator is FGF protein.

36. The method of any one of claim 25-35, wherein the TGF-β activator is selected from the group consisting of a protein, a nucleic acid, a small molecule, and a combination thereof.

37. The method of claim 36, wherein the TGF-β activator is TGF-β protein.

38. The method of claim 36, wherein the TGF-β activator is Activin-A protein.

39. The method of any one of claims 25-38, wherein the WNT activator is selected from the group consisting of a protein, a nucleic acid, a small molecule, and a combination thereof.

40. The method of claim 39, wherein the WNT activator is WNT protein.

41. The method of any one of claims 25-40, wherein the WNT activator is a GSK3 inhibitor.

42. The method of claim 41, wherein the GSK3 inhibitor is CHIR99021.

43. The method of any one of claims 24-42, wherein step (ii) is performed for 1 to 6 days.

44. The method of any one of claims 24-43, wherein step (iii) is performed for 1 to 8 days.

45. The method of any one of claims 25-44, wherein the formative iPSCs produced in step (iii) are cultured in the presence of at least one differentiation factor.

46. The method of claim 45, wherein the at least one differentiation factor is selected from the group consisting of a growth factor, a WNT inhibitor, a INK inhibitor, and a TGF-β inhibitor.

47. The method of claim 46, wherein the growth factor is fibroblast growth factor (FGF), Noggin, or a combination thereof.

48. The method of claim 46 or claim 47, wherein the WNT inhibitor is IWP2.

49. The method of any one of claims 46-48, wherein the TGF-β inhibitor is SB431542.

50. The method of any one of claims 46-48, wherein the INK inhibitor is SP600125.

51. An in vitro culture system comprising:
(a) a population of cells, and
(b) a medium that comprises a fibroblast growth factor (FGF) activator, a transforming growth factor beta (TGF-β) activator, and a WNT activator.

52. The in vitro culture system of claim 50, further comprising a reprogramming agent that modulates expression of at least one reprogramming gene selected from the group consisting of OCT3/4, p53, SOX2, KLF 4, L-MYC, and LIN28.

53. The in vitro culture system of claim 51 or claim 52, wherein the population of cells comprises reproductive cells or somatic cells.

54. The in vitro culture system of any one of claims 51-53, wherein the population of cells comprises blastocysts or embryonic fibroblasts.

55. The in vitro culture system of any one of claims 51-54, wherein the population of reproductive cells are obtained from a subject.

56. The in vitro culture system of claim 55, wherein the subject is selected from the group consisting of a primate, a rodent, and an ungulate.

57. The in vitro culture system of any one of claims 51-56, wherein the FGF activator is selected from the group consisting of a protein, a nucleic acid, a small molecule, and a combination thereof.

58. The in vitro culture system of claim 57, wherein the FGF activator is FGF protein.

59. The in vitro culture system of any one of claims 51-58, wherein the TGF-β activator is selected from the group consisting of a protein, a nucleic acid, a small molecule, and a combination thereof.

60. The in vitro culture system of claim 59, wherein the TGF-β activator is TGF-β protein.

61. The in vitro culture system of claim 59, wherein the TGF-β activator is Activin-A protein.

62. The in vitro culture system of any one of claims 51-61, wherein the WNT activator is selected from the group consisting of a protein, a nucleic acid, a small molecule, and a combination thereof.

63. The in vitro culture system of claim 62, wherein the WNT activator is WNT protein.

64. The in vitro culture system of any one of claims 51-63, wherein the WNT activator is a GSK3 inhibitor.

65. The in vitro culture system of claim 64, wherein the GSK3 inhibitor is CHIR99021.

66. The in vitro culture system of any one of claims 51-65, wherein the medium further comprises at least one additional factor.

67. The in vitro culture system of claim 66, wherein the at least one additional factor is a MEK inhibitor, fetal bovine serum (FBS), leukemia inhibitory factor (LIF), INK inhibitor, or a combination thereof.

68. The in vitro culture system of claim 67, wherein the MEK inhibitor is PD0325901.

69. The in vitro culture system of claim 67, wherein the INK inhibitor is SP600125.

70. The in vitro culture system of any one of claims 42-69, wherein the medium further comprises at least one differentiation factor.

71. The in vitro culture system of claim 70, wherein the at least one differentiation factor is selected from the group consisting of a growth factor, a WNT inhibitor, and a TGF-β inhibitor.

72. The in vitro culture system of claim 71, wherein the growth factor is fibroblast growth factor (FGF), Noggin, or a combination thereof.

73. The in vitro culture system of claim 71 or claim 72, wherein the WNT inhibitor is IWP2.

74. The in vitro culture system of any one of claims 71-73, wherein the TGF-β inhibitor is SB431542.

75. A method for producing formative embryonic stem cells (ESCs), the method comprising:
(i) obtaining a population of reproductive cells from a mouse,
(ii) culturing the population of reproductive cells in a first medium that comprises a MEK inhibitor, and
(iii) culturing the population of reproductive cells in a second medium that comprises a fibroblast growth factor (FGF) activator, a transforming growth factor beta (TGF-β) activator, and a WNT activator, thereby producing formative mouse ESCs.

76. A method for producing formative embryonic stem cells (ESCs), the method comprising:
(i) obtaining a population of reproductive cells from a horse,
(ii) culturing the population of reproductive cells first in a medium that comprises fetal bovine serum (FBS), a fibroblast growth factor (FGF) activator, a transforming growth factor beta (TGF-β) activator, and a WNT activator, and
(iii) culturing the population of reproductive cells in a second medium that comprises the fibroblast growth factor (FGF) activator, the transforming growth factor beta (TGF-β) activator, and the WNT activator, thereby producing formative horse ESCs.

77. A method for producing formative embryonic stem cells (ESCs), the method comprising:
(i) obtaining a population of reproductive cells from a pig,
(ii) culturing the population of reproductive cells first in a medium that comprises fetal bovine serum (FBS), a fibroblast growth factor (FGF) activator, a leukemia inhibitory factor (LIF) activator, a transforming growth factor beta (TGF-fβ) activator, and a WNT activator, and
(iii) culturing the population of reproductive cells in a second medium that comprises the fibroblast growth factor (FGF) activator, the leukemia inhibitory factor (LIF) activator, the transforming growth factor beta (TGF-fβ) activator, and the WNT activator, thereby producing formative pig ESCs.

78. A method for producing formative induced pluripotent stem cells (iPSCs), the method comprising:
(i) obtaining a population of somatic cells from a human,
(ii) culturing the population of somatic cells in a first medium that comprises a reprogramming agent that modulates expression of at least one reprogramming gene selected from the group consisting of OCT3/4, p53, SOX2, KLF4, L-MYC, and LIN28, and
(iii) culturing the population of somatic cells in a second medium that comprises a fibroblast growth factor (FGF) activator, a transforming growth factor beta (TGF-β) activator, a INK inhibitor and a WNT activator,
thereby producing formative human iPSCs.
